(19) United States
(12) Patent Application Publication
(10) Pub. No.: US 2024/0398896 A1
(43) Pub. Date: Dec. 5, 2024

(54) INFLAMMASOME INHIBITION

(71) Applicant: National University of Singapore, Singapore (SG)

(72) Inventors: Lin-Fa Wang, Singapore (SG); Matae Ahn, Singapore (SG); Vivian Chih-Wei Chen, Singapore (SG)

(73) Assignee: National University of Singapore, Singapore (SG)

(21) Appl. No.: 18/695,653

(22) PCT Filed: Sep. 27, 2022
(86) PCT No.: PCT/EP2022/076855

§ 371 Date: Mar. 26, 2024

(30) Foreign Application Priority Data
Sep. 27, 2021 (SG) 10202110660X
Sep. 27, 2021 (SG) 10202110661Y

(51) Int. Cl. A61K 38/17; A61K 47/64; A61P 29/00; C07K 14/47

(52) U.S. Cl. CPC A61K 38/1761; A61K 47/645; A61P 29/00; C07K 14/4747; C07K 2319/10

(57) ABSTRACT

The present disclosure provides ASC inhibitors, compositions comprising said ASC inhibitors, methods of identifying said ASC inhibitors, and therapeutic and prophylactic methods of using said ASC inhibitors.

This application claims priority from SG 10202110660X filed 27 Sep. 2021 and SG 10202110661Y filed 27 Sep. 2021, the contents and elements of which are herein incorporated by reference for all purposes.

### TECHNICAL FIELD

The present disclosure relates to the fields of molecular biology, and the treatment/prevention of disease, in particular disease characterised by aberrant inflammasome activation and/or excessive inflammasome activity.

### BACKGROUND

Inflammasomes are multi-protein signalling complexes that trigger and control the inflammatory responses^{1,10}. The assembly of an inflammasome is initiated by an intracellular pattern-recognition receptor (PRR) through sensing of various highly conserved pathogen-associated molecular patterns (PAMPs) or host-derived danger-associated molecular patterns (DAMPs)¹¹. These PRRs include NOD-like receptors (NLR), such as NLRP3, NLRP1 and NLRC4, and AIM2-like receptor (ALR), such as AIM2 and IFI16. A canonical inflammasome is composed of a NLR/ALR (sensor), a common adaptor and a common effector. Activated NLR or ALR, in most cases, recruit the adaptor protein known as apoptosis-associated speck-like protein containing a CARD (ASC). In activated immune cells like macrophages, cytoplasmic ASC is redistributed into a single ‘ASC speck’, a hallmark of inflammasome assembly and activation^{12,13}. ASC specks subsequently recruit and activate the effector caspase-1, which results in the proteolytic cleavage and secretion of potent pro-inflammatory cytokines IL-1β and IL-18 as well as an inflammatory lytic cell death called pyroptosis. In addition to the cytokines, pyroptosis also triggers the release of inflammatory lipid mediators¹⁴ and ASC specks^{15,16}. Released ASC specks can further propagate the inflammation by extracellular activation of caspase-1/IL-1B and intracellular induction of new ASC specks in surrounding macrophages after phagocytosis^{16,17}. This systemic inflammation triggered by inflammasome activation must be finely controlled. Dysregulated or excessive activation can lead to collateral damage and immune pathology in viral infections, various autoinflammatory/autoimmune diseases, and many age-related metabolic and neurodegenerative diseases including atherosclerosis, diabetes, gout, Parkinson's disease and Alzheimer's disease^{18-20,22}.

The breadth of diseases in which inflammasomes are causally associated is striking, including many of those with a high impact on public health. These findings have triggered increasing efforts in the field to develop pharmacological inhibitors for the inflammasome²¹. However, the only three approved therapies available so far target the downstream cytokine IL-1β with notable disadvantages²². These are antagonistic proteins or antibodies blocking either the IL-1 receptor or IL-1β. All of them require administration by injections with poor penetration of blood-brain barrier. More importantly, they have limited effectiveness because they fail to inhibit many other critical pro-inflammatory factors including IL-18, lipid mediators, ASC specks and pyroptosis. Therefore, inhibitors targeting the effector caspase-1 and upstream are in theory more effective with wider application. Two caspase-1 inhibitors have been identified and subsequently reached up to phase II clinical trials for rheumatoid arthritis and psoriasis but were discontinued due to hepatotoxicity. While these caspase-1 inhibitors are more specific than the pan-caspase inhibitors, they can still have some “off-target” effects on other caspases, especially caspase-4/5 belonging to the caspase-1 subfamily^{23,24}.

More recently, a number of NLRP3 inhibitors have emerged due to the strong inflammatory potential of NLRP3 and its role in many inflammatory diseases^{3,21}. NLRP3 is capable of sensing a wide variety of microbial and damage-related stimuli²¹. The majority of NLRP3 inhibitors have come from phenotypic screening due to lack of high-resolution crystal structures and limited understanding of mechanistic details of NLRP3. Hence, these compounds can be either a direct or indirect inhibitor of NLRP3, and for many of them there is a lack of detailed understanding of their mechanism of action. The best studied and most widely used NLRP3 inhibitor is CP-456,773, also called MCC95025, with the mechanism of action just resolved recently^{26,27}. Although a wide range of preclinical models have been successful, it was discontinued after reaching phase II clinical trials for rheumatoid arthritis due to liver toxicity. While this approach targeting NLRP3 is attractive, it is limited in inflammatory diseases involving tissues or lesions where NLRP3 is not the predominant inflammasome sensor or is only one of the main sensors. For instance, NLRP1, but not NLRP3, is the predominant sensor in human skin and is responsible for certain skin inflammatory diseases and cancers²⁸⁻³⁰. Both NLRP1 and NLRP3 contribute to neuroinflammation in Alzheimer's disease^{31,32}. Recent studies also reveal a critical role of AIM2, an inflammasome sensor for cytosolic DNA, in promoting atherosclerosis³³, causing ischemic brain injury in stroke³⁴ and contributing to virus-induced morbidity/mortality³⁵, in addition to NLRP3. Another limitation of the NLRP3 inhibitors is that they would not suppress extracellular and ‘prionoid’ activity of ASC specks, which amplify inflammation in a NLRP3 independent manner^{15,16}.

ASC is the adaptor linking the sensors and the effector. Therefore, ASC inhibitors would suppress pathological inflammation mediated by multiple key sensors including NLRP3, NLRP1 and AIM2 and hence have wider application and/or better efficacy. They would also block all the caspase-1-dependent inflammatory factors downstream. Besides, they would also prevent the amplification of the inflammatory cascade by extracellular ASC specks. However, there are no existing inhibitors targeting ASC so far. Technical difficulties of targeting ASC include relatively little understanding about the regulation of ASC activation and lack of high-resolution structure for full-length ASC protein. In addition, most inflammasome inhibitors have been derived from phenotypic screening and there is lack of robust high-throughput assays for target-based drug screening of NLRP3 or ASC.

This present invention relates to the treatment and prevention of diseases in which inflammasome activity is pathologically-implicated, through ASC antagonism. The current invention encompasses the novel therapies targeting ASC for multiple inflammasome pathways with application in various human diseases including but not limited to autoinflammatory/autoimmune disorders, infectious diseases, metabolic diseases and neurodegenerative diseases. The current invention also provides a robust high-throughput assay for target based drug screening of ASC and interacting partners of ASC.

Bats, as the only flying mammals, have many unique features, including their long lifespan, low tumorigenesis and hosting many viruses highly lethal in humans without clinical diseases^{4.5}. These observations have led to our investigation into the inflammation control in bats, especially the inflammasomes. Our previous published work demonstrated the dampened inflammasome activation at the sensor level, including NLRP3⁶ and AIM2⁷, and at the effector or cytokine level⁸⁶. Importantly, our present invention reveals that bat ASC2, a potent negative regulator of inflammasomes, is highly expressed in bat cells and significantly suppresses human/mouse inflammasome activation at the adaptor ASC level. 89-amino-acid ASC2, also known as PYRIN-only protein 1 (POP1) or PYRIN domain containing 1 (PYDC1), has only a PYRIN domain without a CARD domain responsible for the downstream recruitment and activation of caspase-1⁹. Human ASC2 is very poorly expressed or not detectable in most human tissues⁹ and inconsistent functional results of potentiating⁹, neutral³⁶ or inhibitory³⁷ effects have been reported. Moreover, ASC2 and the other members of its gene family are believed to be restricted to primates only⁸. However, our study reveals the presence of ASC2 in more than six bat species examined and high endogenous expression is detected especially in immune cells. More importantly, in contrast to human counterpart, bat ASC2 is highly potent in inhibiting human/mouse inflammasomes in vitro and in vivo, suggesting an ASC inhibitor with wide applications in human diseases. This present invention also relates to inhibitors of inflammasomes derived from bat ASC2 and compositions and methods of using such inhibitors. The current invention encompasses the novel therapies targeting ASC for multiple inflammasome pathways with application in various human diseases including but not limited to autoinflammatory/autoimmune disorders, infectious diseases, metabolic diseases and neurodegenerative diseases.

### SUMMARY

In a first aspect, the present disclosure provides a polypeptide comprising or consisting of an amino acid sequence having at least 50% amino acid sequence identity to SEQ ID NO:6, wherein the amino acid sequence of the polypeptide is non-identical to the amino acid sequence of a naturally-occurring protein, and wherein the polypeptide inhibits interaction between ASC and an interaction partner for ASC.

In accordance with various aspects disclosed herein, in some embodiments the polypeptide comprises one or more of: E or D at the position corresponding to position 10 of SEQ ID NO:6; R, K or H at the position corresponding position 37 of SEQ ID NO:6; C, R, K or H at the position corresponding position 61 of SEQ ID NO:6; and G at the position corresponding position 77 of SEQ ID NO:6.

In accordance with various aspects disclosed herein, in some embodiments the polypeptide comprises one or more of: E at the position corresponding to position 10 of SEQ ID NO:6; R at the position corresponding position 37 of SEQ ID NO:6; C at the position corresponding position 61 of SEQ ID NO:6; and G at the position corresponding position 77 of SEQ ID NO:6.

In accordance with various aspects disclosed herein, in some embodiments the polypeptide comprises E or D at the position corresponding to position 10 of SEQ ID NO:6; R, K or H at the position corresponding position 37 of SEQ ID NO:6; C, R, K or H at the position corresponding position 61 of SEQ ID NO:6; and G at the position corresponding position 77 of SEQ ID NO:6.

In accordance with various aspects disclosed herein, in some embodiments the polypeptide comprises E at the position corresponding to position 10 of SEQ ID NO:6; R at the position corresponding position 37 of SEQ ID NO:6; C at the position corresponding position 61 of SEQ ID NO:6; and G at the position corresponding position 77 of SEQ ID NO:6.

In accordance with various aspects disclosed herein, in some embodiments the polypeptide comprises or consists of the amino acid sequence of SEQ ID NO:6.

In accordance with various aspects disclosed herein, in some embodiments the polypeptide further comprises a cell-penetrating peptide.

Also provided is a composition comprising a polypeptide according to the present disclosure, encapsulated in, or immobilised on, a nanoparticle, liposome, nanogel or nanolipogel.

Also provided is a nucleic acid encoding a polypeptide according to the present disclosure.

Also provided is an expression vector, comprising a nucleic acid according to the present disclosure.

Also provided is a cell comprising a polypeptide, a composition, a nucleic acid, or an expression vector according to the present disclosure.

Also provided is a method for producing a polypeptide according to the present disclosure, comprising culturing a cell comprising a nucleic acid according to the present disclosure, or an expression vector according to the present disclosure, under conditions suitable for expression of the polypeptide by the cell.

Also provided is a pharmaceutical composition comprising a polypeptide, a composition, a nucleic acid, an expression vector, or a cell according to the present disclosure, and a pharmaceutically acceptable carrier, diluent, excipient or adjuvant.

Also provided is a polypeptide, a composition, a nucleic acid, an expression vector, a cell, or a pharmaceutical composition according to the present disclosure, for use in a method of medical treatment or prophylaxis.

Also provided is a polypeptide, a composition, a nucleic acid, an expression vector, a cell, or a pharmaceutical composition according to the present disclosure, for use in a method of treating or preventing a disease or condition in which inflammasome activity is pathologically-implicated.

Also provided is the use of a polypeptide, a composition, a nucleic acid, an expression vector, a cell, or a pharmaceutical composition according to the present disclosure, in the manufacture of a medicament for treating or preventing a disease or condition in which inflammasome activity is pathologically-implicated.

Also provided is a method of treating or preventing a disease or condition in which inflammasome activity is pathologically-implicated, comprising administering a therapeutically- or prophylactically-effective amount of a polypeptide, a composition, a nucleic acid, an expression vector, a cell, or a pharmaceutical composition according to the present disclosure, to a subject in need thereof.

In accordance with various aspects disclosed herein, in some embodiments the disease or condition in which inflammasome activity is pathologically-implicated is selected from: an inflammatory disease, a chronic inflammatory disease, a neurodegenerative disease, Alzheimer's disease, Parkinson's disease, an autoimmune disease, multiple sclerosis, rheumatoid arthritis, systemic lupus erythematosus (SLE), an autoinflammatory disease, cryopyrin-associated periodic syndrome (CAPS), familial cold autoinflammatory syndrome (FCAS), Muckle-Wells syndrome (MWS), neonatal onset multisystemic inflammatory disease (NOMID), chronic infantile neurologic cutaneous articular (CINCA), a cancer, myelodysplastic syndrome, a cardiovascular disease, atherosclerosis, acute myocardial infarction, stroke, hypertension, an ocular disease, glaucoma, acute glaucoma, macular degeneration, age-related macular degeneration, a respiratory disease, asthma, severe steroid-resistant asthma, chronic obstructive pulmonary disease (COPD), respiratory disease cause by viral infection, gout, a metabolic disease, obesity-induced insulin resistance, diabetes, type 1 diabetes, type 2 diabetes, non-alcoholic fatty liver disease (NAFLD), silicosis, crystal nephropathy, a skin disease, acne, atopic dermatitis, psoriasis, vitiligo, contact hypersensitivity, inflammatory disease caused by viral infection, inflammatory bowel disease, graft-versus-host disease and traumatic brain injury.

Also provided is a method for inhibiting an inflammasome, comprising introducing into a cell, in vitro or in vivo:
(i) a polypeptide comprising or consisting of an amino acid sequence having at least 50% amino acid sequence identity to SEQ ID NO: 6, 7, 8, 9, 10, 11, 12, 13, 14, 15, 16, 17, 21, 22, 23, 24, 25, 26, 27, 28, 29, 30, 31, 32, 33, 34, 35 or 36 which inhibits interaction between ASC and an interaction partner for ASC;
(ii) polypeptide according to (i), wherein the polypeptide further comprises a cell-penetrating peptide;
(iii) a composition comprising a polypeptide according to (i) or (ii), encapsulated in, or immobilised on, a nanoparticle, liposome, nanogel or nanolipogel;
(iv) a nucleic acid encoding a polypeptide according to (i) or (ii); or
(v) an expression vector comprising a nucleic acid according to (iv).

Also provided is the use of:
(i) a polypeptide comprising or consisting of an amino acid sequence having at least 50% amino acid sequence identity to SEQ ID NO: 6, 7, 8, 9, 10, 11, 12, 13, 14, 15, 16, 17, 21, 22, 23, 24, 25, 26, 27, 28, 29, 30, 31, 32, 33, 34, 35 or 36 which inhibits interaction between ASC and an interaction partner for ASC;
(ii) polypeptide according to (i), wherein the polypeptide further comprises a cell-penetrating
(iii) a composition comprising a polypeptide according to (i) or (ii), encapsulated in, or immobilised on, a nanoparticle, liposome, nanogel or nanolipogel;
(iv) a nucleic acid encoding a polypeptide according to (i) or (ii); or
(v) an expression vector comprising a nucleic acid according to (iv);
in an in vitro or in vivo method for inhibiting an inflammasome.

### DESCRIPTION

The present disclosure is broadly based on the inventors' finding that inhibition of ASC blocks inflammasome-mediated inflammation in a broad range of models of inflammation.

Inhibition of ASC finds use in the treatment/prevention of diseases and conditions characterised by aberrant inflammasome activation and/or excessive inflammasome activity.

Inhibition of ASC by ASC inhibitors of the present disclosure advantageously provides for the inhibition upstream of effectors of inflammasome-mediated inflammation (e.g. IL-1β, IL-18, etc.), and thereby provides for effective, broad-spectrum inhibition of inflammasome-mediated inflammation. Inhibition of ASC by ASC inhibitors of the present disclosure also advantageously provides for the inhibition of processes mediated by a variety of different types of inflammasome, including NLRP1, NLRP3, AIM2, IFI16 and pyrin inflammasomes.

The present disclosure is also broadly based on the inventors unexpected finding that the bat homologue of the human PYDC1 gene (which encodes the protein ASC2) encodes a potent inhibitor of inflammasome activity. Bat ASC2 is shown to associate with and competitively inhibit the function of ASC. The inventors introduced four amino acid substitutions in the amino acid sequence of human ASC2 to arrive at a ‘batized’ version of the protein which was found to strongly inhibit inflammasome activity.

The ASC2-derived inhibitors of the present disclosure find use in the treatment/prevention of diseases and conditions characterised by aberrant inflammasome activation and/or excessive inflammasome activity.

The ASC2-derived inhibitors of the present disclosure advantageously provide for the inhibition upstream of effectors of inflammasome-mediated inflammation (e.g. IL-1B, IL-18, etc.), and thereby provide for effective, broad-spectrum inhibition of inflammasome-mediated inflammation. The ASC2-derived inhibitors of the present disclosure also advantageously provide for the inhibition of processes mediated by a variety of different types of inflammasome, including NLRP1, NLRP3, AIM2, IFI16 and pyrin inflammasomes.

### Inflammasome Structure and Function

The present disclosure is concerned with inflammasome inhibition, in particular inflammasome inhibition using ASC inhibitors, e.g. ASC2-derived polypeptides.

Inflammasome structure and function is reviewed e.g. in Sharma and de Alba, Int J Mol Sci. (2021) 22 (2): 872 and Lu and Wu, The FEBS Journal (2015) 282 (3): 435-44 and Kelley et al., Int J Mol Sci. (2019) 20 (13): 3328, all of which are hereby incorporated by reference in their entirety. Inflammasomes are cytosolic multiprotein complexes involved in the initiation of the innate immune response to pathogens and allergens. Pathogen-associated molecular patterns (PAMPs) and danger-associated molecular patterns (DAMPs) arising from physical/chemical insult are recognised by pathogen recognition receptors (PRRs), resulting in inflammasome activation, which in turn initiates inflammation by triggering the production of proinflammatory cytokines including IL-1β and IL-18.

Inflammasomes typically comprise sensor, adaptor and effector components, which associate through homotypic interaction between death domains. In the presence of external stimuli or specific ligands, PRR sensor proteins such as NLRP3 oligomerise and associate with the adaptor molecule ASC (Apoptosis-associated speck-like protein containing a caspase-activation and recruitment domain (CARD)), which in turn recruits pro-caspase 1 (through CARD-CARD homotypic recognition), resulting in caspase-1 activation. Homotypic interactions between the CARD domains of ASC proteins and caspase-1 results in proteolytic cleavage of pro-caspase-1 and the formation of active caspase-1, which in turn cleaves pro-protein precursors of IL-1B and IL-18 to their biologically active forms. IL-1B induces the expression of genes that control fever, pain, vasodilatation, and hypotension, among others. IL-18 is a costimulatory cytokine that mediates adaptive immunity, and is required for IFN-γ production. Active caspase-1 also cleaves gasdermin D (GSDMD), leading to pyroptosis.

Several different types of PRR have been reported to form inflammasomes: the nucleotide-binding oligomerization domain (NOD), leucine-rich repeat (LRR)-containing (NLR) family members NLRP1, NLRP2, NLRP3, NLRP6, NLRP7, NLRP12 and NLRC4; ALR family members absent-in-melanoma 2 (AIM2) and gamma-interferon-inducible protein 16 (IFI16); and pyrin.

For some inflammasomes such as those comprising NLRP1, NLRP3, AIM2, IFI16 or pyrin, the bipartite adaptor protein apoptosis-associated speck-like protein containing a caspase-activation and recruitment domain (ASC) facilitates the recruitment of pro-caspase-1 to the inflammasome complex.

Inflammasomes are commonly referred to by reference to the identity of their constituent PRR sensor protein. By way of illustration, the inflammasome comprising NLRP3, ASC and pro-caspase-1 is typically referred to as the NLRP3 inflammasome, and similarly the inflammasome comprising AIM2, ASC and pro-caspase-1 is referred to as the AIM2 inflammasome.

The best-studied inflammasome is the NLRP3 inflammasome, which is important for immune responses to bacterial, fungal, and viral pathogens and parasites. However, aberrant NLRP3 inflammasome activation or excessive activity is also implicated in the pathogenesis of several inflammatory disorders, such as cryopyrin-associated periodic syndromes (CAPS), Alzheimer's disease, diabetes, gout, autoinflammatory diseases, and atherosclerosis.

NLRP3 is a tripartite protein, comprising an amino-terminal pyrin domain (PYD), a central nucleotide-binding and oligomerization domain (NOD, sometimes also referred to as the NACHT domain), and a C-terminal leucine-rich repeat (LRR) domain. Interaction between NLRP3 and the adaptor ASC protein occurs through homotypic interaction between the PYD domains of the respective proteins, and is important for inflammasome assembly. The NOD domain has ATPase activity, and is required for NLRP3 oligomerization following activation.

The AIM2 inflammasome comprises the ALR protein AIM2, which comprises a HIN200 domain and an N-terminal PYD domain. AIM2 is activated via its HIN200 domain by the cytosolic presence of dsDNA, and associates with ASC through homotypic interaction between the PYD domains of the respective proteins.

In some embodiments, the inflammasome according to the present disclosure is selected from: an NLR family protein inflammasome (e.g. an NLRP1, NLRP2, NLRP3, NLRP6, NLRP7, NLRP12 or NLRC4 inflammasome); a ALR family protein inflammasome (e.g. an AIM2 inflammasome or an IFI16 inflammasome); and a pyrin inflammasome. In some embodiments, the inflammasome is an inflammasome comprising ASC. In some embodiments, the inflammasome is selected from: an NLRP3 inflammasome, an AIM2 inflammasome, an IFI16 inflammasome, an NLRP1 inflammasome, an NLRC4 inflammasome and a pyrin inflammasome. In some embodiments, the inflammasome is an inflammasome comprising a PRR comprising a PYD domain. In some embodiments, the inflammasome is an inflammasome comprising NLRP3, AIM2, NLRP1, IFI16, NLRC4 or pyrin. In some embodiments, the inflammasome is an inflammasome comprising NLRP3, AIM2, or NLRC4.

### ASC

Apoptosis associated speck like protein containing CARD (ASC) structure and function is described in Agrawal and Jha, Molecular Biology Reports (2020) 47:3077-3096, which is hereby incorporated by reference in its entirety.

Alterative splicing of the mRNA transcribed from human PYCARD results in the production of three different isoforms. Isoform 1 has the amino acid sequence shown in SEQ ID NO:1. Isoform 2 (shown in SEQ ID NO:2) lacks positions 93-111 of SEQ ID NO:1. Isoform 3 (shown in SEQ ID NO:3) lacks positions 26-85 of SEQ ID NO:1.

The 195 amino acid sequence of human ASC isoform 1 is shown in SEQ ID NO:1. N-terminal positions 1-91 of SEQ ID NO:1 form a Pyrin (PYD) domain (SEQ ID NO:4), and C-terminal positions 107-195 form a CARD domain (SEQ ID NO:5).

In this specification, reference to ‘ASC’ encompasses: human ASC, homologues of human ASC (i.e. encoded by the genome of a non-human animal), isoforms of human ASC and isoforms of homologues of human ASC.

A homologue of human ASC may be from a non-human mammal (e.g. a therian, placental, epitherian, preptotheria, archontan, yinpterochiropteran (e.g. a pteropodid (e.g. Pteropus alecto), a rhinolophid (e.g. Rhinolophus ferrumequinum or Rhinolophus sinicus) or a hipposiderid (e.g. Hipposideros armiger)), yangochiropteran (e.g. a phyllostomid (e.g. Phyllostomus discolor), a vespertilionid (e.g. Eptesicus fuscus) or a Mormoopid (e.g. Pteronotus parnellii), or primate (e.g. a non-human primate, a non-human hominid (e.g. Pan troglodytes), an indriid (e.g. Propithecus coquereli), or a cebid (e.g. Saimiri boliviensis)).

Homologues of human ASC may optionally be characterised as having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of human ASC.

Isoforms of human ASC and isoforms of homologues of human ASC may optionally be characterised as having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of the reference protein (i.e. human ASC or the homologue of human ASC).

In some embodiments, ASC according to present disclosure comprises or consists of an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:1, 2 or 3.

### ASC2

ASC2 (also known as PYRIN domain containing 1 (PYDC1)) is the protein encoded in humans by the gene PYDC1. The 89 amino acid sequence of human ASC2 is shown in SEQ ID NO:7, and forms a Pyrin (PYD) domain.

In this specification, reference to ‘ASC2’ encompasses: human ASC2, homologues of human ASC2 (i.e. encoded by the genome of a non-human animal), isoforms of human ASC2 and isoforms of homologues of human ASC2.

A homologue of human ASC2 may be from a non-human mammal (e.g. a therian, placental, epitherian, preptotheria, archontan, yinpterochiropteran (e.g. a pteropodid (e.g. Pteropus alecto), a rhinolophid (e.g. Rhinolophus ferrumequinum or Rhinolophus sinicus) or a hipposiderid (e.g. Hipposideros armiger), yangochiropteran (e.g. a phyllostomid (e.g. Phyllostomus discolor), a vespertilionid (e.g. Eptesicus fuscus) or a Mormoopid (e.g. Pteronotus parnellii), or primate (e.g. a non-human primate, a non-human hominid (e.g. Pan troglodytes), an indriid (e.g. Propithecus coquereli), or a cebid (e.g. Saimiri boliviensis)).

In some embodiments, a homologue of human ASC2 may be ASC2 from a bat. In some embodiments, a homologue of human ASC2 may be ASC2 from a yinpterochiropteran (e.g. a pteropodid (e.g. Pteropus alecto), a rhinolophid (e.g. Rhinolophus ferrumequinum or Rhinolophus sinicus) or a hipposiderid (e.g. Hipposideros armiger), yangochiropteran (e.g. a phyllostomid (e.g. Phyllostomus discolor), a vespertilionid (e.g. Eptesicus fuscus) or a Mormoopid (e.g. Pteronotus parnellii))

Homologues of human ASC2 may optionally be characterised as having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of human ASC2.

Isoforms of human ASC2 and isoforms of homologues of human ASC2 may optionally be characterised as having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of the reference protein (i.e. human ASC2 or the homologue of human ASC2).

In some embodiments, ASC2 according to present disclosure comprises or consists of an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO: 6, 7, 8, 9, 10, 11, 12, 13, 14, 15, 16, 17, 21, 22, 23, 24, 25, 26, 27, 28, 29, 30, 31, 32, 33, 34, 35 or 36. In some embodiments, ASC2 according to present disclosure comprises or consists of an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO: 6, 7, 8, 9, 10, 11, 12, 13, 14, 15, 16, or 17.

### Inhibition of ASC

The present invention is concerned with inhibition of ASC. That is, the invention is concerned with inhibition of the expression and/or activity of ASC and the downstream functional consequences thereof.

Inhibition of ASC encompasses decreased/reduced expression (gene and/or protein expression) of ASC and/or decreased/reduced activity of ASC, relative to the level of expression/activity observed in the absence of inhibition. “Inhibition” may herein also be referred to as “antagonism”.

In accordance with the present disclosure, inhibition of ASC may be characterised by one of more of the following (relative to the uninhibited state):
reduced expression (e.g. gene and/or protein expression) of ASC;
reduced level of RNA encoding ASC;
reduced transcription of nucleic acid encoding ASC;
increased degradation of RNA encoding ASC;
reduced level of ASC protein;
reduced post-transcriptional processing (e.g. splicing, translation, post-translational processing) of RNA encoding ASC;
increased degradation of ASC protein;
reduced level of an ASC function;
inhibition of interaction between ASC and an interaction partner for ASC;
inflammasome inhibition; and/or
inhibition of inflammasome-mediated inflammation.

Gene expression can be determined by means well known to the skilled person. The level of RNA encoding ASC can be determined e.g. by techniques such as RT-qPCR, northern blot, etc.

A reduction in the level of RNA encoding ASC may e.g. be the result of reduced transcription of nucleic acid encoding ASC, or increased degradation of RNA encoding ASC.

Reduced transcription of nucleic acid encoding ASC may be a consequence of inhibition of assembly and/or activity of factors required for transcription of the DNA encoding ASC. Increased degradation of RNA encoding ASC may be a consequence of increased enzymatic degradation of RNA encoding ASC, e.g. as a consequence of RNA interference (RNAi), and/or reduced stability of RNA encoding ASC.

Protein expression can be determined by means well known to the skilled person. The level of an ASC protein can be determined e.g. by antibody-based methods including western blot, immunohisto/cytochemistry, flow cytometry, ELISA, ELISPOT, or by reporter-based methods.

A reduction in the level of an ASC protein may e.g. be the result of a reduction in the level of RNA encoding ASC, reduced post-transcriptional processing of RNA encoding ASC, or increased degradation of ASC protein.

Reduced post-transcriptional processing of ASC may e.g. be reduced splicing of pre-mRNA encoding ASC to mature mRNA encoding ASC, reduced translation of mRNA encoding ASC, or reduced post-translational processing of ASC.

Reduced splicing of pre-mRNA encoding ASC to mature mRNA encoding ASC may be a consequence of inhibition of assembly and/or activity of factors required for splicing. Reduced translation of mRNA encoding ASC may be a consequence of inhibition of assembly and/or activity of factors required for translation. Reduced post-translational processing (e.g. enzymatic processing, folding) of ASC may be a consequence of inhibition of assembly and/or activity of factors required for post-translational processing of ASC.

Increased degradation of ASC protein may be a consequence of increased enzymatic (e.g. protease-mediated) degradation of ASC protein.

In some embodiments, inhibition of ASC may be characterised by a reduced level of an ASC function. An ASC function may be any functional property of ASC e.g. interaction with an interaction partner.

An interaction partner for ASC may be any polypeptide which associates with ASC. An interaction partner for ASC may interact with ASC to form a polypeptide complex. Association between ASC and an interaction partner for ASC may involve covalent interaction (e.g. disulfide bonding) and/or non-covalent interaction (e.g. electrostatic interaction (e.g. ionic bonding, hydrogen bonding), Van der Waals forces).

In some embodiments, an interaction partner for ASC is a PYD domain-containing protein. In some embodiments, an interaction partner for ASC is a PYD domain-containing PRR. In some embodiments, an interaction partner for ASC is ASC (ASC is known to form homomultimers).

In some embodiments, an interaction partner for ASC is selected from ASC, NLRP3, AIM2, NLRP1, IFI16, NLRC4 and pyrin. In some embodiments, an interaction partner for ASC is selected from NLRP3, AIM2, NLRP1, IFI16, NLRC4 and pyrin. In some embodiments, an interaction partner for ASC is selected from NLRP3, AIM2, and NLRC4.

In some embodiments, an interaction partner for ASC is a CARD domain-containing protein. In some embodiments, an interaction partner for ASC is pro-caspase-1. In some embodiments, an interaction partner for ASC is ASC (ASC is known to form homomultimers).

Inhibition of interaction between ASC and an interaction partner for ASC can be identified e.g. by detection of a reduction in the level of interaction between ASC and the interaction partner for ASC, relative to a control, uninhibited condition. The ability of proteins to interact can be analysed by methods well known to the skilled person, such as co-immunoprecipitation, and resonance energy transfer (RET) assays. In some embodiments, the inhibition of interaction between ASC and an interaction partner for ASC can be identified by target-based high-throughput screening as described in Example 7.

Inhibition of ASC function can also be evaluated by analysis of one or more correlates of ASC function. That is, ASC function can be evaluated by analysis of downstream functional consequences of ASC function. For example, inhibition of ASC function can be identified by detection of a decreased level of IL-1β, IL-18, caspase-1 activity, pyroptosis, ASC speck formation and/or inflammasome activity.

In some embodiments inhibition of ASC may be characterised by inflammasome inhibition. Inflammasome inhibition may be a consequence of inhibition of inflammasome assembly, disruption of the inflammasome, and/or inhibition of inflammasome activity/function.

Inhibition of formation of an inflammasome may be achieved through inhibition of interaction between constituent proteins of an inflammasome. For example, inhibition of formation of an inflammasome may be achieved through inhibition of interaction between ASC and an interaction partner for ASC (e.g. ASC, a PYD domain-containing PRR (e.g. NLRP3, AIM2, NLRP1, IFI16 or pyrin), or pro-caspase-1).

Disruption of an inflammasome may be achieved through displacement of a constituent protein of an inflammasome from an inflammasome. For example, disruption of an inflammasome may be achieved through displacement of an interaction partner for ASC (e.g. a PYD domain-containing PRR (e.g. NLRP3, AIM2, NLRP1, IFI16 or pyrin), or pro-caspase-1) from an inflammasome.

In some embodiments, inflammasome inhibition comprises inhibiting one or more functions of an inflammasome (e.g. an inflammasome as described hereinabove). Inflammasome inhibition may be characterised by a reduced level of a function of an inflammasome. In some embodiments, inflammasome inhibition may be inferred by detection of a reduced level of a correlate of inflammasome function.

In some embodiments, a function of an inflammasome may be selected from: caspase-1 activation, IL-1B activation, IL-18 activation and pyroptosis. Inflammasome function may be analysed e.g. using an in vitro assay of inflammasome activity as described in the experimental Examples herein.

Such assays may include (i) culturing macrophages (e.g. THP-1 cell-derived macrophages, BMDMs, iMACs) in vitro, (ii) treating the cells in such a way as to activate an inflammasome (e.g. culturing the cells in the presence of LPS, nigericin or MSU crystals; transfecting the cells with poly(dA:dT) or flagellin; infecting the cells with virus (e.g. RNA virus such as IAV, ZIKV, PRV3M), and (iii) measuring correlates of inflammasome activity (e.g. secreted IL-1B or IL-18 (e.g. as determined by ELISA), ASC speck formation or pyroptosis (e.g. as determined by LDH release)).

In some embodiments inhibition of ASC may be characterised by inhibition of inflammasome-mediated inflammation. Inflammasome-mediated inflammation may be evaluated e.g. in vivo in an animal model of inflammasome-mediated inflammation. Suitable models include e.g. the MSU crystal-induced model of peritonitis, and the virus-induced models of inflammation described in the experimental examples.

### ASC Inhibitors

Aspects of the present invention comprise inhibition of ASC using an inhibitor of ASC.

An “inhibitor of ASC” refers to any agent capable of inhibiting ASC expression and/or function. Such agents may be effectors of (i.e. may directly or indirectly cause) inhibition of ASC. Agents capable of inhibiting ASC may be referred to herein as ASC inhibitors. ASC inhibitors may also be referred to herein as antagonists of ASC/ASC antagonists.

In some embodiments, an inhibitor of ASC may display one or more of the following properties:
reducing expression (e.g. gene and/or protein expression) of ASC;
reducing the level of RNA encoding ASC;
reducing transcription of nucleic acid encoding ASC;
increasing degradation of RNA encoding ASC;
reducing the level of ASC protein;
reducing post-transcriptional processing (e.g. splicing, translation, post-translational processing) of RNA encoding ASC;
increasing degradation of ASC protein;
binding to ASC;
binding to an interaction partner of ASC;
binding to a PYD domain;
binding to a CARD domain;
reducing the interaction between ASC and an interaction partner for ASC;
reducing the level of an ASC function;
inflammasome inhibition; and/or
inhibition of inflammasome-mediated inflammation.

A given agent may be evaluated for the properties recited in the preceding paragraph using suitable assays. The assays may be e.g. in vitro assays, optionally cell-based assays or cell-free assays. The assays may be e.g. in vivo assays, i.e. performed in non-human animals.

Agents capable of reducing gene expression of ASC (e.g. reducing the level of RNA encoding ASC, reducing transcription of nucleic acid encoding ASC and/or increasing degradation of RNA encoding ASC) may be identified using assays comprising detecting the level of RNA encoding ASC, e.g. by RT-qPCR. Such assays may comprise treating cells/tissue with the agent, and subsequently comparing the level of RNA encoding ASC in such cells/tissue to the level of RNA encoding ASC in cells/tissue of an appropriate control condition (e.g. untreated/vehicle-treated cells/tissue).

Agents capable of reducing protein expression of ASC (e.g. reducing the level of ASC protein, increasing degradation of ASC protein) may be identified using assays comprising detecting the level of ASC protein, e.g. using antibody/reporter-based methods (western blot, ELISA, immunohisto/cytochemistry, etc.). Such assays may comprise treating cells/tissue with the agent, and subsequently comparing the level of ASC protein in such cells/tissue to the level of ASC protein in cells/tissue of an appropriate control condition (e.g. untreated/vehicle-treated cells/tissue).

Agents capable of binding to ASC, binding to an interaction partner of ASC, binding to a PYD domain, or binding to a CARD domain may be identified using assays comprising ELISA, Surface Plasmon Resonance (SPR; see e.g. Hearty et al., Methods Mol Biol (2012) 907:411-442), Bio-Layer Interferometry (see e.g. Lad et al., (2015) J Biomol Screen 20 (4): 498-507), flow cytometry, immunoblot (e.g. western blot), co-immunoprecipitation, and resonance energy transfer (RET) assays.

Agents capable of reducing interaction between ASC and an interaction partner for ASC may be identified using assays comprising detecting the level of interaction between ASC and an interaction partner for ASC, e.g. using antibody/reporter-based methods. The level of interaction between ASC and an interaction partner for ASC can be analysed e.g. using resonance energy transfer techniques (e.g. FRET, BRET), or methods analysing a correlate of interaction between ASC and the interaction partner. Assays may comprise treating cells/tissue with the agent, and subsequently comparing the level of interaction between ASC and an interaction partner for ASC in such cells/tissue to the level of interaction between ASC and the interaction partner for ASC in cells/tissue of an appropriate control condition (e.g. untreated/vehicle-treated cells/tissue). The level of interaction between ASC and an interaction partner for ASC can also be analysed e.g. using techniques such as ELISA, surface plasmon resonance or biolayer interferometry analysis. Assays may comprise comparing the level of interaction between ASC and an interaction partner for ASC in the presence of the agent to the level of interaction between ASC and the interaction partner for ASC in an appropriate control condition (e.g. the absence of the agent).

Agents capable of reducing the level of an ASC function can be evaluated by analysis of one or more correlates of ASC function. That is, ASC function can be evaluated by analysis of downstream functional consequences of ASC function. For example, inhibition of ASC function can be identified by detection of a decreased level of IL-1B, IL-18, caspase-1 activity, pyroptosis, ASC speck formation and/or inflammasome activity.

Agents capable of inhibiting inflammasome formation and/or activity may be identified using appropriate assays, e.g. using an in vitro assay of inflammasome activity. Cells may be treated with a putative inflammasome-inhibiting agent prior to, or during, treatment to activate an inflammasome in such an assay. Inflammasome inhibition may be inferred where treatment with an agent is determined to result in a reduction in the level of a correlate of inflammasome activity as compared to the level observed in the absence of the agent (or the level observed in the presence of a control agent known not to inhibit inflammasome activity).

Such assays may include (i) culturing macrophages (e.g. THP-1 cell-derived macrophages, BMDMs, IMACs) in vitro, (ii) treating the cells in such a way as to activate an inflammasome (e.g. culturing the cells in the presence of LPS, nigericin or MSU crystals; transfecting the cells with poly(dA:dT) or flagellin; infecting the cells with virus (e.g. RNA virus such as IAV, ZIKV, PRV3M)), and (iii) measuring correlates of inflammasome activity (e.g. secreted IL-1B or IL-18 (e.g. as determined by ELISA), ASC speck formation or pyroptosis (e.g. as determined by LDH release)).

Agents capable of inhibiting inflammasome-mediated inflammation may be identified using appropriate assays, e.g. in vivo in an animal model of inflammasome-mediated inflammation. Suitable models include e.g. the MSU crystal-induced model of peritonitis, and the virus-induced models of inflammation described in the experimental examples.

In some embodiments, an ASC inhibitor is selected from: an ASC-binding molecule, an ASC target-binding molecule, or a molecule capable of reducing expression of ASC.

An “ASC-binding molecule” refers to a molecule which is capable of binding to ASC. In some embodiments an ASC-binding molecule binds to a polypeptide according to SEQ ID NO:1, SEQ ID NO:2 and/or SEQ ID NO:3.

An “ASC target-binding molecule” refers to a molecule which is capable of binding to an interaction partner for ASC (e.g. an interaction partner for ASC as described herein, such as NLRP3, AIM2, NLRP1, IFI16, NLRC4, pyrin, pro-caspase-1 etc.).

In some embodiments, the ASC-binding molecule or ASC target-binding molecule binds to a PYD domain from ASC or an ASC interacting partner, e.g. the PYD domain from ASC, NLRP3, AIM2, NLRP1, IFI16, NLRC4 or pyrin. In some embodiments, the ASC-binding molecule or ASC target-binding molecule binds to a CARD domain in ASC or an ASC interacting partner, e.g. the CARD domain in pro-caspase-1.

Such binding molecules can be identified using any suitable assay for detecting binding of a molecule to the relevant factor (i.e. ASC, or the interaction partner for ASC). Such assays may comprise detecting the formation of a complex between the relevant factor and the molecule.

In some embodiments the binding agents may be identified using the assay described in Example 7.

ASC-binding molecules and ASC target-binding molecules may display specific binding to the relevant factor (i.e. ASC, or the interaction partner for ASC). As used herein, “specific binding” refers to binding which is selective, and which can be discriminated from non-specific binding to non-target molecules. An ASC-binding molecule that specifically binds to ASC preferably binds to ASC with greater affinity, and/or with greater duration than it binds to other, non-target molecules; such binding molecules may be described as being “specific for” ASC. An ASC target-binding molecule that specifically binds to an interaction partner for ASC preferably binds to the interaction partner for ASC with greater affinity, and/or with greater duration than it binds to other, non-target molecules; such binding molecules may be described as being “specific for” the interaction partner for ASC.

In some embodiments an ASC-binding molecule/ASC target-binding molecule inhibits the ability of ASC to bind to an interaction partner for ASC. In some embodiments an ASC-binding molecule/ASC target-binding molecule behaves as a competitive inhibitor of interaction between ASC and an interaction partner for ASC. In some embodiments an ASC-binding molecule/ASC target-binding molecule may act through competitive inhibition of homotypic interaction between the PYD domain of ASC and a PYD domain-containing interaction partner therefor. In some embodiments an ASC-binding molecule/ASC target-binding molecule may act through competitive inhibition of homotypic interaction between the CARD domain of ASC and a CARD domain-containing interaction partner therefor. The binding molecule may occupy, or otherwise reduce access to, a region of ASC required for binding to an interaction partner for ASC, or may occupy, or otherwise reduce access to, a region of an interaction partner for ASC required for binding to ASC. In some embodiments an ASC-binding molecule/ASC target-binding molecule may occupy or otherwise inhibit access of PYD domain-containing interaction partners to the PYD domain of ASC, preventing their association. In some embodiments an ASC-binding molecule/ASC target-binding molecule may occupy or otherwise inhibit access of CARD domain-containing interaction partners to the CARD domain of ASC, preventing their association.

The ability of an ASC-binding molecule/ASC target-binding molecule to inhibit interaction between ASC and an interaction partner for ASC can be evaluated e.g. by analysis of interaction in the presence of, or following incubation of one or both of the interaction partners with, the ASC-binding molecule/ASC target-binding molecule. An example of a suitable assay to determine whether a given binding agent is capable of inhibiting interaction between ASC and an interaction partner for ASC is a competition ELISA. A further example of a suitable assay to determine whether a given binding agent is capable of inhibiting the interaction between ASC and an interaction partner for ASC is a high-throughput screening assay as described in Example 7.

Agents capable of binding to ASC or an interacting partner of ASC may be of any kind, but in some embodiments the agent may be an antibody, an antigen-binding fragment thereof, a polypeptide, a peptide, a nucleic acid, an oligonucleotide, an aptamer or a small molecule. The agents may be provided in isolated or purified form, or may be formulated as a pharmaceutical composition or medicament.

ASC-binding molecules and ASC target-binding molecules include peptides/polypeptides, e.g. peptide aptamers, thioredoxins, monobodies, anticalin, Kunitz domains, avimers, knottins, fynomers, atrimers, DARPins, affibodies, nanobodies (i.e. single-domain antibodies (sdAbs)) affilins, armadillo repeat proteins (ArmRPs), OBodies and fibronectin-reviewed e.g. in Reverdatto et al., Curr Top Med Chem. 2015; 15 (12): 1082-1101, which is hereby incorporated by reference in its entirety (see also e.g. Boersma et al., J Biol Chem (2011) 286:41273-85 and Emanuel et al., Mabs (2011) 3:38-48). ASC-binding molecules and ASC target-binding molecules include peptides/polypeptides that can be identified by screening of libraries of the relevant peptides/polypeptides.

ASC-binding peptides/polypeptides and ASC target-binding peptides/polypeptides include antibodies, or an antigen-binding fragment thereof. An “antibody” is used herein in the broadest sense, and encompasses monoclonal antibodies, polyclonal antibodies, monospecific antibodies, multispecific antibodies (e.g., bispecific antibodies), and fragments and derivatives thereof (e.g. Fv, scFv, Fab, scFab, F(ab′)₂, Fab₂, diabodies, triabodies, scFv-Fc, minibodies, single domain antibodies (e.g. VhH), etc.), as long as they display binding to the relevant target molecule.

In view of today's techniques in relation to monoclonal antibody technology, antibodies can be prepared to most antigens. The antigen-binding portion may be a part of an antibody (for example a Fab fragment) or a synthetic antibody fragment (for example a single chain Fv fragment [ScFv]). Monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger et al (1988, 8th International Biotechnology Symposium Part 2, 792-799). Monoclonal antibodies (mAbs) are particularly useful in the methods of the invention, and are a homogenous population of antibodies specifically targeting a single epitope on an antigen.

Polyclonal antibodies are also useful in the methods of the invention. Monospecific polyclonal antibodies are preferred. Suitable polyclonal antibodies can be prepared using methods well known in the art.

Antigen-binding fragments of antibodies, such as Fab and Fab2 fragments may also be used/provided as can genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

Antibodies and antigen-binding fragments according to the present disclosure comprise the complementarity-determining regions (CDRs) of an antibody which is capable of binding to the relevant target molecule (i.e. ASC/interacting partner of ASC).

The skilled person is well aware of techniques for producing antibodies suitable for therapeutic use in a given species/subject. For example, procedures for producing antibodies suitable for therapeutic use in humans are described in Park and Smolen, Advances in Protein Chemistry (2001) 56:369-421 (hereby incorporated by reference in its entirety).

Antibodies to a given target protein (e.g. ASC or interacting partner of ASC) can be raised in model species (e.g. rodents, lagomorphs), and subsequently engineered in order to improve their suitability for therapeutic use in a given species/subject. For example, one or more amino acids of monoclonal antibodies raised by immunisation of model species can be substituted to arrive at an antibody sequence which is more similar to human germline immunoglobulin sequences (thereby reducing the potential for anti-xenogenic antibody immune responses in the human subject treated with the antibody). Modifications in the antibody variable domains may focus on the framework regions in order to preserve the antibody paratope. Antibody humanisation is a matter of routine practice in the art of antibody technology, and is reviewed e.g. in Almagro and Fransson, Frontiers in Bioscience (2008) 13:1619-1633, Safdari et al., Biotechnology and Genetic Engineering Reviews (2013) 29 (2): 175-186 and Lo et al., Microbiology Spectrum (2014) 2 (1), all of which are hereby incorporated by reference in their entirety. The requirement for humanisation can be circumvented by raising antibodies to a given target protein (e.g. ASC or interacting partner of ASC) in transgenic model species expressing human immunoglobulin genes, such that the antibodies raised in such animals are fully-human (described e.g. in Brüggemann et al., Arch Immunol Ther Exp (Warsz) (2015) 63 (2): 101-108, which is hereby incorporated by reference in its entirety).

Phage display techniques may also be employed to the identification of antibodies to a given target protein (e.g. ASC or interacting partner of ASC), and are well known to the skilled person. The use of phage display for the identification of fully human antibodies to human target proteins is reviewed e.g. in Hoogenboom, Nat. Biotechnol. (2005) 23, 1105-1116 and Chan et al., International Immunology (2014) 26 (12): 649-657, which are hereby incorporated by reference in their entirety.

ASC-binding peptides/polypeptides and ASC target-binding peptides/polypeptides also include peptide/polypeptide interaction partners for the relevant factor.

ASC-binding peptide/polypeptide interaction partners may be based on an interaction partner for ASC, and may e.g. comprise a ASC-binding fragment of an interaction partner for ASC. ASC target-binding peptide/polypeptide interaction partners may be based on ASC, and may e.g. comprise a ASC target-binding fragment of ASC. Such agents may behave as ‘decoy’ molecules, and preferably display competitive inhibition of interaction between ASC and an interaction partner for ASC and/or ASC-mediated function.

An ASC-binding peptide/polypeptide may be an ASC2-derived polypeptide. An “ASC2-derived polypeptide” refers to a polypeptide comprising or consisting of an amino acid sequence having at least 50%, preferably one of 55%, 60%, 65%, 70%, 75%, 80%, 85%, 90%, 91%, 92%, 93%, 94%, 95%, 96%, 97%, 98%, 99% or greater amino acid sequence identity to the amino acid sequence of ASC2 (SEQ ID NOs 7-17, 21-36). An ASC-binding peptide may comprise or consist of an amino acid sequence having at least 50%, preferably one of 55%, 60%, 65%, 70%, 75%, 80%, 85%, 90%, 91%, 92%, 93%, 94%, 95%, 96%, 97%, 98%, 99% or greater amino acid sequence identity to the amino acid sequence of SEQ ID NO: 6.

Peptides capable of binding to ASC are described in Pal et al., Scientific Reports (2019) 9:4913, which is hereby incorporated by reference in its entirety.

ASC-binding molecules and ASC target-binding molecules include aptamers. Nucleic acid aptamers are reviewed e.g. in Zhou and Rossi, Nat Rev Drug Discov. 2017 16 (3): 181-202, and may be identified and/or produced by the method of Systematic Evolution of Ligands by Exponential enrichment (SELEX), or by developing SOMAmers (slow off-rate modified aptamers) (Gold L et al. (2010) PLOS ONE 5 (12):e15004). Aptamers and SELEX are described in Tuerk and Gold, Science (1990) 249 (4968): 505-10, and in WO 91/19813. Nucleic acid aptamers may comprise DNA and/or RNA, and may be single stranded or double stranded. They may comprise chemically modified nucleic acids, for example in which the sugar and/or phosphate and/or base is chemically modified. Such modifications may improve the stability of the aptamer or make the aptamer more resistant to degradation and may include modification at the 2′ position of ribose. Nucleic acid aptamers may be chemically synthesised, e.g. on a solid support. Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, typically iodine. The cycle may then be repeated to assemble the aptamer (e.g., see Sinha, N. D.; Biernat, J.; McManus, J.; Köster, H. Nucleic Acids Res. 1984, 12, 4539; and Beaucage, S. L.; Lyer, R. P. (1992). Tetrahedron 48 (12): 2223). Peptide aptamers and methods for their generation and identification are reviewed in Reverdatto et al., Curr Top Med Chem. (2015) 15 (12): 1082-101, which is hereby incorporated by reference in its entirety.

ASC-binding molecules and ASC target-binding molecules include small molecules. ASC-binding small molecules and ASC target-binding small molecules can be identified by screening of small molecule libraries.

As used herein, a “small molecule” refers to a low molecular weight (<1000 daltons, typically between ˜300-700 daltons) organic compound.

A small molecule, MCC950 capable of binding to NLRP3 (i.e. an ASC target-binding molecule) is described in Coll, R. C. et al., Nat. Med. (2015) 21:248-255.

A “molecule capable of reducing expression of ASC” refers to a molecule which is capable of reducing gene and/or protein expression of ASC. In some embodiments the molecule reduces or prevents the expression of a polypeptide according to SEQ ID NO:1, SEQ ID NO:2 and/or SEQ ID NO:3.

Repression of expression of ASC or an isoform thereof will preferably result in a decrease in the quantity of ASC expressed by a cell/tissue/organ/organ system/subject. For example, in a given cell the repression of ASC by administration of a suitable nucleic acid will result in a decrease in the level of expression relative to an untreated cell. Repression may be partial. Preferred degrees of repression are at least 50%, more preferably one of at least 60%, 70%, 80%, 85% or 90%. A level of repression between 90% and 100% is considered a ‘silencing’ of expression or function. Gene and protein expression may be determined as described herein or by methods in the art that are well known to a skilled person.

Such agents may be of any kind. In some embodiments a molecule capable of reducing expression of ASC is an inhibitory nucleic acid.

In some embodiments, the inhibitory nucleic acid is an antisense nucleic acid. In some embodiments the inhibitory nucleic acid is an antisense oligonucleotide (ASO). Antisense oligonucleotides are preferably single-stranded, and bind by complementary sequence binding, to a target oligonucleotide, e.g. mRNA.

In view of the known nucleic acid sequences for ASC, oligonucleotides may be designed to repress or silence the expression of ASC, or particular isoforms thereof. The mRNA sequence for human ASC is shown in NCBI Reference Sequence NM_013258.5 (SEQ ID NO:18).

Oligonucleotides designed to repress or silence the expression of ASC, or particular isoforms thereof, may have substantial sequence identity to a portion of ASC/the relevant isoform, or the complementary sequence thereto.

In some embodiments, the inhibitory nucleic acid reduces ASC expression by RNA interference (RNAi). RNAi involves inhibition of gene expression and translation by targeted neutralisation of mRNA molecules. In some embodiments, the inhibitory nucleic acid is small interfering RNA (siRNA), a short hairpin RNA (shRNA), or a micro RNA (miRNA).

A role for the RNAi machinery and small RNAs in targeting of heterochromatin complexes and epigenetic gene silencing at specific chromosomal loci has been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of SIRNA. RNAi based therapeutics have been progressed into Phase I, II and III clinical trials for a number of indications (Nature 2009 Jan. 22; 457 (7228): 426-433).

In the art, such RNA sequences are termed “short or small interfering RNAs” (siRNAs) or “microRNAs” (miRNAs) depending on their origin. Both types of sequence may be used to down-regulate gene expression by binding to complementary RNAs and either triggering mRNA elimination (RNAi) or arresting mRNA translation into protein. siRNAs are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

siRNAs are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNAs are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed in John et al, PLOS Biology, 11 (2), 1862-1879, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo) nucleotides, typically a UU of dTdT 3′ overhang. Based on the disclosure provided herein, the skilled person can readily design suitable siRNA and miRNA sequences, for example using resources such the Ambion siRNA finder. siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (see for example Myers (2003) Nature Biotechnology 21:324-328). The longer dsRNA molecule may have symmetric 3′ or 5′ overhangs, e.g. of one or two (ribo) nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17, 1340-5, 2003).

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector. shRNAs may be produced within a cell by transfecting the cell with a vector encoding the shRNA sequence under control of a RNA polymerase III promoter such as the human H1 or 7SK promoter or a RNA polymerase Il promoter. Alternatively, the shRNA may be synthesised exogenously (in vitro) by transcription from a vector. The shRNA may then be introduced directly into the cell. Preferably, the shRNA molecule comprises a partial sequence of ASC. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

In some embodiments, the inhibitory nucleic acid is a splice-switching oligonucleotide (SSO). Splice switching oligonucleotides are reviewed e.g. in Haves and Hastings, Nucleic Acids Res. (2016) 44 (14): 6549-6563, which is hereby incorporated by reference in its entirety. SSOs disrupt the normal splicing of target RNA transcripts by blocking the RNA-RNA base-pairing and/or protein-RNA binding interactions that occur between components of the splicing machinery and pre-mRNA. SSOs may be employed to reduce the number/proportion of mature mRNA transcripts encoding the ASC isoform that it is intended to inhibit. SSOs generally comprise alterations to oligonucleotide sugar-phosphate backbones to prevent RNAse H degradation, and may comprise include e.g. phosphorodiamidate morpholino (PMOs), peptide nucleic acid (PNA), locked nucleic acid (LNA), and/or 2′O-methyl (2′OMe) and 2′-O-methoxyethyl (MOE) ribose modifications.

Inhibitory nucleic acids may be made recombinantly by transcription of a nucleic acid sequence, preferably contained within a vector. In some embodiments inhibitory nucleic acids are produced within a cell, e.g. by transcription from a vector. Vectors encoding such molecules may be introduced into cells in any of the ways known in the art. Optionally, expression of the RNA sequence can be regulated using a tissue specific (e.g. heart, liver, or kidney specific) promoter.

Inhibitory nucleic acids may also be synthesized using standard solid or solution phase synthesis techniques which are known in the art.

In some embodiments, inhibition of ASC may comprise modification of a cell(s) to reduce or prevent expression of ASC. In some embodiments inhibition of ASC comprises modifying nucleic acid encoding ASC. The modification causes the cell to have a reduced level of gene and/or protein expression of ASC as compared to an unmodified cell.

In some embodiments inhibition of ASC may comprise modifying a gene encoding ASC. In some embodiments inhibition of ASC comprises introducing an insertion, substitution or deletion into a nucleic acid sequence encoding ASC.

In some embodiments inhibition of ASC comprises introducing a modification which reduces or prevents the expression of a polypeptide according to SEQ ID NO:1 from the modified nucleic acid sequence. In some embodiments inhibition of ASC comprises modifying a cell to comprise an ASC allele which does not encode an amino acid sequence according to SEQ ID NO:1. In some embodiments inhibition of ASC comprises modifying a cell to lack nucleic acid encoding a polypeptide according to SEQ ID NO:1.

In some embodiments inhibition of ASC comprises modifying ASC to introduce a premature stop codon in the sequence transcribed from ASC. In some embodiments inhibition of ASC comprises modifying ASC to encode a truncated and/or non-functional ASC polypeptide. In some embodiments inhibition of ASC comprises modifying ASC to encode an ASC polypeptide which is misfolded and/or degraded.

Modification of a nucleic acid encoding ASC can be achieved in a variety of ways known to the skilled person, including modification of the target nucleic acid by homologous recombination, and target nucleic acid editing using site-specific nucleases (SSNs).

Suitable methods may employ targeting by homologous recombination, which is reviewed, for example, in Mortensen, Curr Protoc Neurosci. (2007) Chapter 4: Unit 4.29 and Vasquez et al., PNAS 2001, 98 (15): 8403-8410 both of which are hereby incorporated by reference in their entirety. Targeting by homologous recombination involves the exchange of nucleic acid sequence through crossover events guided by homologous sequences.

The methods employ target nucleic acid editing using SSNs. Gene editing using SSNs is reviewed e.g. in Eid and Mahfouz, Exp Mol Med. 2016 October; 48 (10): e265, which is hereby incorporated by reference in its entirety. Enzymes capable of creating site-specific double strand breaks (DSBs) can be engineered to introduce DSBs to target nucleic acid sequence(s) of interest. DSBs may be repaired by either error-prone non-homologous end-joining (NHEJ), in which the two ends of the break are rejoined, often with insertion or deletion of nucleotides. Alternatively, DSBs may be repaired by highly homology-directed repair (HDR), in which a DNA template with ends homologous to the break site is supplied and introduced at the site of the DSB.

SSNs capable of being engineered to generate target nucleic acid sequence-specific DSBs include zinc-finger nucleases (ZFNs), transcription activator-like effector nucleases (TALENs) and clustered regularly interspaced palindromic repeats/CRISPR-associated-9 (CRISPR/Cas9) systems.

ZFN systems are reviewed e.g. in Umov et al., Nat Rev Genet. (2010) 11 (9): 636-46, which is hereby incorporated by reference in its entirety. ZFNs comprise a programmable Zinc Finger DNA-binding domain and a DNA-cleaving domain (e.g. a FokI endonuclease domain). The DNA-binding domain may be identified by screening a Zinc Finger array capable of binding to the target nucleic acid sequence.

TALEN systems are reviewed e.g. in Mahfouz et al., Plant Biotechnol J. (2014) 12 (8): 1006-14, which is hereby incorporated by reference in its entirety. TALENs comprise a programmable DNA-binding TALE domain and a DNA-cleaving domain (e.g. a FokI endonuclease domain). TALEs comprise repeat domains consisting of repeats of 33-39 amino acids, which are identical except for two residues at positions 12 and 13 of each repeat which are repeat variable di-residues (RVDs). Each RVD determines binding of the repeat to a nucleotide in the target DNA sequence according to the following relationship: “HD” binds to C, “NI” binds to A, “NG” binds to T and “NN” or “NK” binds to G (Moscou and Bogdanove, Science (2009) 326 (5959): 1501).

CRISPR/Cas9 and related systems e.g. CRISPR/Cpf1, CRISPR/C2c1, CRISPR/C2c2 and CRISPR/C2c3 are reviewed e.g. in Nakade et al., Bioengineered (2017) 8 (3): 265-273, which is hereby incorporated by reference in its entirety. These systems comprise an endonuclease (e.g. Cas9, Cpf1 etc.) and the single-guide RNA (sgRNA) molecule. The sgRNA can be engineered to target endonuclease activity to nucleic acid sequences of interest.

In some embodiments, inhibition of ASC employs a site-specific nuclease (SSN) system targeting ASC. The SSN system may be a ZFN system, a TALEN system, CRISPR/Cas9 system, a CRISPR/Cpf1 system, a CRISPR/C2c1 system, a CRISPR/C2c2 system or a CRISPR/C2c3 system.

For example, inhibition of ASC may employ nucleic acid(s) encoding a CRISPR/Cas9 system. The nucleic acid(s) may encode a CRISPR RNA (crRNA) targeting an exon of ASC and a trans-activating crRNA (tracrRNA) for processing the crRNA to its mature form.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate). In some embodiments the cell is an epithelial cell (e.g. HEK293T) or an immune cell (e.g. THP-1).

The nucleotide sequence may be contained in a vector, e.g. an expression vector. A “vector” as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector.

The term “operably linked” may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. The resulting transcript(s) may then be translated into a desired peptide(s)/polypeptide(s).

Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes).

In some embodiments, the vector may be a eukaryotic vector, e.g. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

### Polypeptides of the Disclosure

The present disclosure provides polypeptides.

In some aspects and embodiments, polypeptides of the present disclosure may be referred to as ASC2-derived polypeptides. As used herein, “an ASC2-derived polypeptide” refers to a polypeptide comprising, or consisting of, an amino acid sequence having at least 50%, preferably one of 55%, 60%, 65%, 70%, 75%, 80%, 85%, 90%, 91%, 92%, 93%, 94%, 95%, 96%, 97%, 98%, 99% or greater amino acid sequence identity to the amino acid sequence of ASC2.

In some embodiments, a polypeptide according the present disclosure comprises, or consists of, an amino acid sequence having at least 50%, preferably one of 55%, 60%, 65%, 70%, 75%, 80%, 85%, 90%, 91%, 92%, 93%, 94%, 95%, 96%, 97%, 98%, 99% or greater amino acid sequence identity to the amino acid sequence of SEQ ID NO: 6, 7, 8, 9, 10, 11, 12, 13, 14, 15, 16, 17, 21, 22, 23, 24, 25, 26, 27, 28, 29, 30, 31, 32, 33, 34, 35 or 36. In some embodiments, a polypeptide according the present disclosure comprises, or consists of, an amino acid sequence having at least 50%, preferably one of 55%, 60%, 65%, 70%, 75%, 80%, 85%, 90%, 91%, 92%, 93%, 94%, 95%, 96%, 97%, 98%, 99% or greater amino acid sequence identity to the amino acid sequence of SEQ ID NO: 6, 7, 8, 9, 10, 11, 12, 13, 14, 15, 16, or 17.

In some embodiments, the polypeptides of the present disclosure consist of an amino sequence which is non-identical to the amino acid sequence of a naturally-occurring protein. Herein, a ‘naturally-occurring protein’ refers to a protein which exists in nature. In some embodiments, a naturally-occurring protein may be an endogenous protein encoded by the genome of an individual of a species of animal. In some embodiments, a polypeptide according to the present disclosure may comprise at least one amino acid substitution relative to the amino acid sequence of a naturally-occurring protein.

In some embodiments, a polypeptide according the present disclosure comprises one or more amino acid substitutions relative to the amino acid sequence of ASC2. In some embodiments, an ASC2-derived polypeptide may be referred to as a variant or a mutant of ASC2.

In some embodiments, a polypeptide according the present disclosure comprises, or consists of, an amino acid sequence having at least 50%, preferably one of 55%, 60%, 65%, 70%, 75%, 80%, 85%, 90%, 91%, 92%, 93%, 94%, 95%, 96%, 97%, 98%, 99% or greater amino acid sequence identity to the amino acid sequence of SEQ ID NO:7, 8, 9, 10, 11, 12, 13, 14, 15, 16, 17, 21, 22, 23, 24, 25, 26, 27, 28, 29, 30, 31, 32, 33, 34, 35 or 36, and comprising one or more amino acid substitutions relative to the reference sequence. In some embodiments, a polypeptide according the present disclosure comprises, or consists of, an amino acid sequence having at least 50%, preferably one of 55%, 60%, 65%, 70%, 75%, 80%, 85%, 90%, 91%, 92%, 93%, 94%, 95%, 96%, 97%, 98%, 99% or greater amino acid sequence identity to the amino acid sequence of SEQ ID NO:7, 8, 9, 10, 11, 12, 13, 14, 15, 16, or 17, and comprising one or more amino acid substitutions relative to the reference sequence.

In some embodiments, a polypeptide according the present disclosure comprises more than one amino acid substitution relative to the amino acid sequence of a wildtype ASC2 protein. Where reference is made herein to ‘a wildtype ASC2 protein’ this is to be understood as referring to ASC2 proteins encoded by the most common allele encoding ASC2, in species of animal having a genome encoding an ASC2. By way of illustration, the amino acid sequence of wildtype human ASC2 is shown in SEQ ID NO:7.

In some embodiments, a polypeptide comprises 1, 2, 3, 4, 5, 6, 7, 8, 9, 10, 11, 12, 13, 14, 15, 16, 17, 18, 19 or 20 amino acid substitutions relative to the amino acid sequence of a wildtype ASC2 protein.

In some embodiments, a polypeptide according the present disclosure comprises, or consists of, an amino acid sequence having at least 50%, preferably one of 55%, 60%, 65%, 70%, 75%, 80%, 85%, 90%, 91%, 92%, 93%, 94%, 95%, 96%, 97%, 98%, 99% or greater amino acid sequence identity to the amino acid sequence of SEQ ID NO: 7, 8, 9, 10, 11, 12, 13, 14, 15, 16, 17, 21, 22, 23, 24, 25, 26, 27, 28, 29, 30, 31, 32, 33, 34, 35 or 36, and comprising 1, 2, 3, 4, 5, 6, 7, 8, 9, 10, 11, 12, 13, 14, 15, 16, 17, 18, 19 or 20 amino acid substitutions relative to the reference sequence. In some embodiments, a polypeptide according the present disclosure comprises, or consists of, an amino acid sequence having at least 50%, preferably one of 55%, 60%, 65%, 70%, 75%, 80%, 85%, 90%, 91%, 92%, 93%, 94%, 95%, 96%, 97%, 98%, 99% or greater amino acid sequence identity to the amino acid sequence of SEQ ID NO: 7, 8, 9, 10, 11, 12, 13, 14, 15, 16, or 17, and comprising 1, 2, 3, 4, 5, 6, 7, 8, 9, 10, 11, 12, 13, 14, 15, 16, 17, 18, 19 or 20 amino acid substitutions relative to the reference sequence.

In some embodiments, a polypeptide according the present disclosure comprises, or consists of, an amino acid sequence having at least 50%, preferably one of 55%, 60%, 65%, 70%, 75%, 80%, 85%, 90%, 91%, 92%, 93%, 94%, 95%, 96%, 97%, 98%, 99% or greater amino acid sequence identity to the amino acid sequence of SEQ ID NO:7, and comprising 1, 2, 3, 4, 5, 6, 7, 8, 9, 10, 11, 12, 13, 14, 15, 16, 17, 18, 19 or 20 amino acid substitutions relative to the reference sequence.

In some embodiments, a polypeptide according the present disclosure comprises, or consists of, an amino acid sequence having at least 50%, preferably one of 55%, 60%, 65%, 70%, 75%, 80%, 85%, 90%, 91%, 92%, 93%, 94%, 95%, 96%, 97%, 98%, 99% or greater amino acid sequence identity to the amino acid sequence of SEQ ID NO:7, and comprising one or more (e.g. 1, 2, 3 or 4) of the following: (a) a negatively charged amino acid (e.g. E or D) at the position corresponding to position 10 of SEQ ID NO: 7; (b) a positively charged amino acid (e.g. R, H or K) at the position corresponding to position 37 of SEQ ID NO:7; (c) C, or a positively charged amino acid (e.g. R, H or K), at the position corresponding to position 61 of SEQ ID NO:7; and (d) G at the position corresponding to position 77 of SEQ ID NO:7.

In some embodiments, an amino acid at a given position of an amino acid sequence referred to herein may be substituted with another amino acid. In some embodiments, a positively-charged amino acid (e.g. R, H or K) may be substituted with another positively-charged amino acid. In some embodiments, a negatively-charged amino acid (e.g. D or E) may be substituted with another negatively-charged amino acid. In some embodiments, an amino acid having a polar uncharged side chain (e.g. S, T, C, N or Q) may be substituted with another amino acid having a polar uncharged side chain. In some embodiments, a non-polar amino acid (e.g. G, A, V, P, L, I, M, W or F) may be substituted with another non-polar amino acid. In some embodiments, an amino acid having a hydrophobic side chain (e.g. A, V, I, L, M, F, Y or W) may be substituted with another amino acid having hydrophobic side chain.

In some embodiments, substitution(s) may be functionally conservative. That is, in some embodiments the substitution may not affect (or may not substantially affect) one or more functional properties (e.g. inflammasome inhibition) of the polypeptide comprising the amino acid sequence having the substitution relative to a polypeptide comprising the equivalent unsubstituted sequence.

Herein, a position or region of an amino acid sequence which ‘corresponds’ to a specified position/region of a reference amino acid sequence can be identified by sequence alignment of the subject sequence to the reference sequence, e.g. using sequence alignment software such as ClustalOmega (Söding, J. 2005, Bioinformatics 21, 951-960). By way of illustration, position 10 of SEQ ID NO:6 corresponds to position 10 of SEQ ID NO:7.

In some embodiments, a polypeptide according the present disclosure comprises, or consists of, an amino acid sequence having at least 50%, preferably one of 55%, 60%, 65%, 70%, 75%, 80%, 85%, 90%, 91%, 92%, 93%, 94%, 95%, 96%, 97%, 98%, 99% or greater amino acid sequence identity to the amino acid sequence of SEQ ID NO:7, and comprising one or more (e.g. 1, 2, 3 or 4) of the following: (a) E at the position corresponding to position 10 of SEQ ID NO:7; (b) R at the position corresponding to position 37 of SEQ ID NO:7; (c) C or R at the position corresponding to position 61 of SEQ ID NO:7; and (d) G at the position corresponding to position 77 of SEQ ID NO:7.

In some embodiments, a polypeptide according the present disclosure comprises, or consists of, an amino acid sequence having at least 50%, preferably one of 55%, 60%, 65%, 70%, 75%, 80%, 85%, 90%, 91%, 92%, 93%, 94%, 95%, 96%, 97%, 98%, 99% or greater amino acid sequence identity to the amino acid sequence of SEQ ID NO:7, and comprising one or more (e.g. 1, 2, 3 or 4) of the following: (a) E at the position corresponding to position 10 of SEQ ID NO:7; (b) R at the position corresponding to position 37 of SEQ ID NO:7; (c) C at the position corresponding to position 61 of SEQ ID NO:7; and (d) G at the position corresponding to position 77 of SEQ ID NO:7. In some embodiments, a polypeptide according the present disclosure comprises, or consists of, an amino acid sequence having at least 50%, preferably one of 55%, 60%, 65%, 70%, 75%, 80%, 85%, 90%, 91%, 92%, 93%, 94%, 95%, 96%, 97%, 98%, 99% or greater amino acid sequence identity to the amino acid sequence of SEQ ID NO:7, and comprising one or more (e.g. 1, 2, 3 or 4) of the following: (a) E at the position corresponding to position 10 of SEQ ID NO:7; (b) R at the position corresponding to position 37 of SEQ ID NO:7; (c) R at the position corresponding to position 61 of SEQ ID NO:7; and (d) G at the position corresponding to position 77 of SEQ ID NO:7.

### Functional Properties of the Polypeptides of the Disclosure

The polypeptides of the present disclosure may be characterised by reference to certain functional properties.

In some embodiments, a polypeptide according to the present disclosure displays one or more of the following properties:
binding to a PYD domain;
binding to ASC;
inhibition of interaction between PYD-domain containing-proteins;
inhibition of interaction between ASC and an interaction partner for ASC;
inflammasome inhibition; and
inhibition of inflammasome-mediated inflammation.

The polypeptides described herein preferably display binding to a PYD domain and/or a PYD domain-containing protein (e.g. ASC). In some embodiments, the polypeptide of the present disclosure binds to a polypeptide comprising, or consisting of, an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:4.

In some embodiments, the polypeptide of the present disclosure binds to ASC, e.g. ASC as described hereinabove. In some embodiments, the polypeptide binds to human ASC. In some embodiments, the polypeptide binds to a polypeptide comprising, or consisting of, an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:1, 2 or 3.

In some embodiments, binding by the polypeptides described herein to a PYD domain, a PYD domain-containing protein and/or ASC is achieved via homotypic protein-protein interaction. Homotypic protein-protein interactions may involve non-covalent interaction. Non-covalent interactions include e.g. electrostatic, π-effects, van der Waals forces, and hydrophobic effects. Binding may be reversible or irreversible.

The ability of a given polypeptide to bind to a reference polypeptide can be determined by analysis according to methods known in the art, such as by ELISA, Surface Plasmon Resonance (SPR; see e.g. Hearty et al., Methods Mol Biol (2012) 907:411-442), Bio-Layer Interferometry (see e.g. Lad et al., (2015) J Biomol Screen 20 (4): 498-507), flow cytometry, immunoblot (e.g. western blot), co-immunoprecipitation, and resonance energy transfer (RET) assays.

The ability of a given polypeptide to bind to ASC can be determined by analysis as described in the experimental examples.

In some embodiments, the polypeptides of the disclosure bind to a PYD domain, a PYD domain-containing protein, and/or ASC with greater affinity than the affinity with which they bind to a polypeptide not comprising a PYD domain. In some embodiments, the binding of the polypeptide to a protein not comprising a PYD domain is less than about 10% of the level of binding displayed by the polypeptide to a PYD domain, a PYD domain-containing protein, and/or ASC (e.g. as determined by ELISA, SPR or BLI).

In some embodiments, the polypeptides of the present disclosure bind to ASC (e.g. human ASC) with greater affinity, and/or with greater duration, than a reference polypeptide binds to ASC (e.g. human ASC).

In some embodiments, the polypeptides of the present disclosure bind to ASC (e.g. human ASC) with an affinity and/or duration which is more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times, ≥10 times, ≥100 times or ≥1000 times the affinity and/or duration of a reference polypeptide. In some embodiments, the reference polypeptide is human ASC2 (e.g. human ASC2, as described hereinabove).

The affinity of binding to ASC for a polypeptide described herein may be determined by analysis according to methods known in the art, such as Surface Plasmon Resonance (SPR), Bio-Layer Interferometry (BLI), immunoblot (e.g. western blot), co-immunoprecipitation, and nuclear magnetic resonance (NMR).

In some embodiments, the polypeptide of the present disclosure is an inhibitor of interaction between a PYD domain, a PYD domain-containing protein, and/or ASC and an interaction partner therefor. In some embodiments the polypeptide is an inhibitor of interaction between ASC and an interaction partner for ASC.

In some embodiments, an interaction partner for ASC is PYD domain-containing protein. In some embodiments, an interaction partner for ASC is a PYD domain-containing PRR. In some embodiments, an interaction partner for ASC is ASC (ASC is known to form homomultimers).

In some embodiments, an interaction partner for ASC is selected from ASC, NLRP3, AIM2, NLRP1, IFI16, NLRC4 and pyrin. In some embodiments, an interaction partner for ASC is selected from NLRP3, AIM2, NLRP1, IFI16, NLRC4 and pyrin. In some embodiments, an interaction partner for ASC is selected from NLRP3, AIM2, and NLRC4.

Polypeptides which inhibit interaction between a PYD domain, a PYD domain-containing protein, and/or ASC and an interaction partner therefor may be described as antagonists of such interaction.

The ability of a given polypeptide to inhibit interaction between two factors can be determined for example by analysis of interaction in the presence of, or following incubation of one or both of the interaction partners with, the polypeptide. An example of a suitable assay to determine whether a given polypeptide inhibits interaction between two interaction partners is a competition ELISA assay. A polypeptide which inhibits a given interaction (e.g. between interaction between a PYD domain, a PYD domain-containing protein, and/or ASC and an interaction partner therefor) is identified by the observation of a reduction/decrease in the level of interaction between the interaction partners in the presence of—or following incubation of one or both of the interaction partners with—the polypeptide, as compared to the level of interaction in the absence of the polypeptide (or in the presence of an appropriate control polypeptide). Suitable analysis can be performed in vitro, e.g. using recombinant interaction partners or using cells expressing the interaction partners. Cells expressing interaction partners may do so endogenously, or may do so from nucleic acid introduced into the cell. For the purposes of such assays, one or both of the interaction partners and/or the polypeptide may be labelled or used in conjunction with a detectable entity for the purposes of detecting and/or measuring the level of interaction.

The ability of a polypeptide to inhibit interaction between a PYD domain, a PYD domain-containing protein, and/or ASC and an interaction partner therefor can be analysed as described in the experimental examples. The ability of a polypeptide to inhibit interaction between interaction partners can also be determined by analysis of the downstream functional consequences of such interaction, e.g. inflammasome activity and/or the functional consequences thereof.

In some embodiments, the polypeptide of the present disclosure inhibits interaction between ASC and an interaction partner for ASC (e.g. ASC, NLRP3, AIM2, NLRP1, IFI16, NLRC4 or pyrin) to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of interaction between the interaction partners in the absence of the polypeptide (or in the presence of an appropriate control polypeptide).

In some embodiments, the polypeptide of the present disclosure binds to the same region or an overlapping region of a PYD domain, a PYD domain-containing protein, and/or ASC as the region bound by an interaction partner therefor. In some embodiments the polypeptide binds to the same region or an overlapping region of ASC as the region bound by an interaction partner for ASC. In some embodiments, the polypeptide is a competitive inhibitor of interaction between a PYD domain, a PYD domain-containing protein, and/or ASC and an interaction partner therefor.

In some embodiments, the polypeptide of the present disclosure binds to ASC in the region corresponding to SEQ ID NO:4.

Whether a given polypeptide (e.g. a polypeptide of the present disclosure) binds to the same or an overlapping region of a given target (e.g. ASC) as a reference molecule (e.g. an interaction partner for ASC) can be evaluated, for example, by analysis of (i) interaction between the target and reference molecule in the absence of the polypeptide, and (ii) interaction between the target and reference molecule in the presence of the polypeptide, or following incubation of the target with the polypeptide. Determination of a reduced level of interaction between the target and the reference molecule following analysis according to (ii) as compared to (i) might support an inference that the polypeptide and reference molecule bind to the same or an overlapping region of the target. Suitable assays for such analysis include e.g. competition ELISA assays and epitope binning assays.

In some embodiments, polypeptides of the present disclosure bind to ASC (e.g. the PYD domain thereof) with greater affinity than the affinity with which an interaction partner for ASC binds to ASC.

In some embodiments, the K_{D} for interaction between a polypeptide of the present disclosure and ASC is lower than the K_{D} for interaction between an interaction partner for ASC and ASC. In some embodiments, the Kₒₙ for interaction between a polypeptide of the present disclosure and ASC is higher than the Kₒₙ for interaction between an interaction partner for ASC and ASC. In some embodiments, the K_{off} for interaction between a polypeptide of the present disclosure and ASC is lower than the K_{off} for interaction between an interaction partner for ASC and ASC. The binding kinetics for a given interaction can be determined using methods well known in the art, such as SPR and BLI analysis.

In some embodiments the polypeptide of the present disclosure is an inflammasome inhibitor. In some embodiments, the polypeptide inhibits formation of an inflammasome (i.e. inhibits inflammasome assembly). In some embodiments, the polypeptide disrupts an inflammasome. In some embodiments, the polypeptide inhibits inflammasome activity/function.

In some embodiments, the polypeptide of the present disclosure is an ASC inhibitor. An ASC inhibitor may be characterised by the ability to inhibit an activity of ASC, or an activity of a protein complex comprising ASC.

Inhibition of formation of an inflammasome may be achieved through inhibition of interaction between constituent proteins of an inflammasome. For example, inhibition of formation of an inflammasome may be achieved through inhibition of interaction between ASC and an interaction partner for ASC (e.g. ASC, or a PYD domain-containing PRR (e.g. NLRP3, AIM2, NLRP1, IFI16 or pyrin)).

The molecular basis of the ASC and/or inflammasome inhibition by the polypeptides of the present disclosure may be through competitive inhibition of homotypic interaction between the PYD domain of ASC and a PYD domain-containing interaction partners therefor. Polypeptides of the present disclosure may occupy or otherwise inhibit access of PYD domain-containing interaction partners to the PYD domain of ASC, preventing their association.

Disruption of an inflammasome may be achieved through displacement of a constituent protein of an inflammasome from an inflammasome. For example, disruption of an inflammasome may be achieved through displacement of an interaction partner for ASC (e.g. a PYD domain-containing PRR, e.g. NLRP3, AIM2, NLRP1, IFI16 or pyrin) from an inflammasome.

In some embodiments, inflammasome inhibition comprises inhibiting one or more functions of an inflammasome (e.g. an inflammasome as described hereinabove). Inflammasome inhibition may be characterised by a reduced level of a function of an inflammasome. In some embodiments, inflammasome inhibition may be inferred by detection of a reduced level of a correlate of inflammasome function.

In some embodiments, a function of an inflammasome may be selected from: caspase-1 activation, IL-1B activation, IL-18 activation and pyroptosis. Inflammasome function may be analysed e.g. using an in vitro assay of inflammasome activity as described in the experimental Examples herein.

Such assays may include (i) culturing macrophages (e.g. THP-1 cell-derived macrophages, BMDMs, IMACs) in vitro, (ii) treating the cells in such a way as to activate an inflammasome (e.g. culturing the cells in the presence of LPS, nigericin or MSU crystals; transfecting the cells with poly(dA:dT) or flagellin; infecting the cells with virus (e.g. RNA virus such as IAV, ZIKV, PRV3M), and (iii) measuring correlates of inflammasome activity (e.g. secreted IL-1B or IL-18 (e.g. as determined by ELISA), ASC speck formation or pyroptosis (e.g. as determined by LDH release)).

Polypeptides may be evaluated for inflammasome inhibition in such assays. Cells may be treated with a putative inflammasome-inhibiting polypeptide prior to, or during, treatment to activate an inflammasome in such an assay. Inflammasome inhibition may be inferred where treatment with a polypeptide is determined to result in a reduction in the level of a correlate of inflammasome activity as compared to the level observed in the absence of the polypeptide (or the level observed in the presence of a control polypeptide known not to inhibit inflammasome activity).

In some embodiments, the polypeptide of the present disclosure inhibits inflammasome activity (e.g. as determined by the level of secreted IL-1B, ASC speck formation, or LDH release) to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of inflammasome activity observed in the absence of the polypeptide (or in the presence of an appropriate control polypeptide), in an assay of inflammasome activity.

In some embodiments, the polypeptide of the present disclosure inhibits inflammasome-mediated inflammation. Inflammasome-mediated inflammation may be evaluated e.g. in vivo in an animal model of inflammasome-mediated inflammation. Suitable models include e.g. the MSU crystal-induced model of peritonitis, and the virus-induced models of inflammation described in the experimental examples.

In some embodiments, the polypeptide of the present disclosure inhibits inflammasome-mediated inflammation to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of inflammasome-mediated inflammation observed in the absence of treatment with the polypeptide (or in the presence of treatment with an appropriate control polypeptide), in an in vivo assay of inflammasome-mediated inflammation.

### Additional Sequences and Linkers

In some embodiments, polypeptides according to the present disclosure may additionally comprise one or more further amino acids or sequences of amino acids.

In some embodiments, the additional amino acids/sequence of amino acids is provided at the N-terminus of the amino acid sequence of the polypeptide. In some embodiments, the additional amino acids/sequence of amino acids is provided at the C-terminus of the amino acid sequence of the polypeptide. In some embodiments, additional amino acids/sequences of amino acids are provided at the N-terminus and C-terminus of the amino acid sequence of the polypeptide.

Polypeptides according to the disclosure may comprise amino acid sequence(s) to facilitate cellular uptake/entry. The polypeptides may comprise amino acid sequence(s) to facilitate traversal of cell membrane and/or entry to the cytosolic space.

In some embodiments the polypeptides comprise an amino acid sequence encoding a cell-penetrating peptide. Cell penetrating peptides and polypeptides are described e.g. in Xie et al., Front. Pharmacol. (2020) 11:697, which is hereby incorporated by reference in its entirety. In some embodiments, the cell-penetrating peptide is or comprises a cell-penetrating peptide disclosed in Xie et al., Front. Pharmacol. (2020) 11:697. In some embodiments, the cell-penetrating peptide is or comprises a protein transduction domain, trojan peptide, arginine-rich peptide or a vectocell peptide.

In some embodiments, the cell-penetrating peptide is selected from: a cationic cell-penetrating peptide (e.g. TAT, R8, DPV3, DPV6, DPV1047, K16ApoE, penatratin, R9-TAT or RVG-9R), an amphipathic cell-penetrating peptide (e.g. pVEC, PEP-1, ARF (19-31), MPG, MAP, P28, transportan, TP10, or cell-penetrating artificial mitochondria-targeting peptide (CAMP)), or a hydrophobic cell-penetrating peptide (e.g. Bip4, C105Y, melittin or gH625). In some embodiments, the cell-penetrating peptide is TAT.

In some embodiments, the polypeptides additionally comprise an amino acid sequence encoding a domain/moiety capable of binding to a target cell of interest or an antigen thereof. In some embodiments, the amino acid sequence encodes an antibody/antigen-binding fragment thereof, a peptide aptamer, or a ligand for a cell surface molecule/a cell surface molecule-binding fragment thereof.

Polypeptides according to the disclosure may comprise amino acid sequence(s) to facilitate expression, folding, trafficking, processing, purification or detection of the polypeptide. For example, polypeptides according to the present disclosure may comprise an amino acid sequence encoding a His, (e.g. 6×His), Myc, GST, MBP, FLAG, HA, E, or Biotin tag. In some embodiments the polypeptide comprises an amino acid sequence encoding a His tag.

In some embodiments, a polypeptide according to the present disclosure further comprises a detectable moiety, e.g. a fluorescent, luminescent, immuno-detectable, radio, chemical, nucleic acid or enzymatic label.

In some embodiments, a polypeptide according to the present disclosure further comprises an amino acid sequence encoding a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides.

The amino acid sequence encoding a signal peptide may be present at the N-terminus of the polypeptide, and may be present in the newly-synthesised polypeptide. The signal peptide provides for efficient trafficking and secretion of the polypeptide. Signal peptides are often removed by cleavage, and thus are not comprised in the mature polypeptide secreted from the cell expressing the polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8:785-786) or Signal-BLAST (Frank and Sippl, 2008 Bioinformatics 24:2172-2176).

In some embodiments, a polypeptide according to the present disclosure comprises one or more linker sequences, e.g. between amino acid sequences of the polypeptide. Linker sequences may be provided at one or both ends of a specified amino acid sequence of the polypeptide.

Linker sequences are known to the skilled person, and are described, for example in Chen et al., Adv Drug Deliv Rev (2013) 65 (10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65 (10): 1357-1369. Flexible linker sequences often comprise high proportions of glycine and/or serine residues.

In some embodiments, the linker sequence comprises at least one glycine residue and/or at least one serine residue. In some embodiments the linker sequence consists of glycine and serine residues. In some embodiments, the linker sequence comprises one or more copies (e.g. in tandem) of the sequence motif G4S. In some embodiments, the linker sequence has a length of 1-2, 1-3, 1-4, 1-5, 1-10, 1-15, 1-20, 1-25, or 1-30 amino acids.

### Labels and Conjugates

In some embodiments, a polypeptide according to the present disclosure additionally comprises a label or conjugate.

In some embodiments, the detectable moiety may be a fluorescent label, phosphorescent label, luminescent label, immuno-detectable label (e.g. an epitope tag), radiolabel, chemical, nucleic acid or enzymatic label. The polypeptide may be covalently or non-covalently labelled with the detectable moiety.

Fluorescent labels include e.g. fluorescein, rhodamine, allophycocyanin, eosine and NDB, green fluorescent protein (GFP), chelates of rare earths such as europium (Eu), terbium (Tb) and samarium (Sm), tetramethyl rhodamine, Texas Red, 4-methyl umbelliferone, 7-amino-4-methyl coumarin, Cy3, and Cy5. Radiolabels include radioisotopes such as Iodine¹²³, Iodine¹²⁵, Iodine¹²⁶, Iodine¹³¹, Iodine¹³³ Bromine⁷⁷, Technetium^{99m}, Indium¹¹¹, Indium^{113m}, Gallium⁶⁷, Gallium⁶⁸, Ruthenium⁹⁵, Ruthenium⁹⁷, Ruthenium¹⁰³, Ruthenium¹⁰⁵, Mercury²⁰⁷, Mercury²⁰³, Rhenium^{99m}, Rhenium¹⁰¹, Rhenium¹⁰⁵, Scandium⁴⁷, Tellurium^{121m}, Tellurium^{122m}, Tellurium^{125m}, Thulium¹⁶⁵, Thuliuml¹⁶⁷, Thulium¹⁶⁸, Copper⁶⁷, Fluorine¹⁸, Yttrium⁹⁰, Palladium¹⁰⁰, Bismuth²¹⁷ and Antimony²¹¹. Luminescent labels include e.g. radioluminescent, chemiluminescent (e.g. acridinium ester, luminol, isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers. Enzymatic labels include e.g. peroxidase, alkaline phosphatase, glucose oxidase, beta-galactosidase and luciferase.

In some embodiments, the polypeptide is conjugated to a chemical moiety. The chemical moiety may be a moiety for providing a therapeutic effect. Antibody-drug conjugates are reviewed e.g. in Parslow et al., Biomedicines. 2016 September; 4 (3): 14. In some embodiments, the chemical moiety may be a drug moiety (e.g. a cytotoxic agent).

### Nucleic Acids, Vectors

The present disclosure provides a nucleic acid encoding a polypeptide according to the present disclosure. In some embodiments the nucleic acid comprises or consists of DNA and/or RNA.

The present disclosure also provides a vector comprising the nucleic acid according to the present disclosure.

The nucleic acid may be, or may be comprised in, a vector, e.g. an expression vector. The nucleotide sequence of the nucleic acid may be contained in a vector, e.g. an expression vector. A “vector” as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector according to the present disclosure.

The term “operably linked” may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. The resulting transcript(s) may then be translated into a desired peptide/polypeptide.

Nucleic acids and vectors according to the present disclosure may be provided in purified or isolated form, i.e. isolated/purified from other nucleic acid, or naturally-occurring biological material.

The nucleic acid and/or vector according to the present disclosure is preferably provided for introduction into a cell. Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 or Morgan and Boyerinas, Biomedicines 2016 4, 9, which are both hereby incorporated by reference in its entirety.

In some embodiments, the vector may be a eukaryotic vector, e.g. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression. In some embodiments, the viral vector may be a lentiviral, retroviral, adenoviral, or Herpes Simplex Virus vector.

In some embodiments, a vector is selected based on tropism for a cell type/tissue/organ to which it is desired to deliver the nucleic acid, e.g. a cell type/tissue/organ affected by the disease to be treated/prevented in accordance with the present disclosure (i.e. cells/tissue/an organ in which the symptoms of the disease manifest). In some embodiments, a vector is selected based on tropism for a macrophage or a precursor thereof (e.g. a monocyte, a macrophage/DC progenitor cell or a myeloid progenitor cell).

In some embodiments it is desired to deliver nucleic acid encoding a inflammasome inhibitor to a macrophage, and vectors having a tropism for mast cells may be employed in such embodiments.

In some embodiments the nucleic acid/vector comprises one or more sequences for controlling expression of the nucleic acid. Accordingly, in some embodiments the nucleic acid/vector comprises a control element for inducible expression of the nucleic acid.

A sequence for controlling expression of the nucleic acid may provide for expression of the nucleic acid by cells of a particular type or tissue. For example, expression may be under the control of a cell type- or tissue-specific promoter. Promoters for cell type- or tissue-specific expression of a nucleic acid in accordance with the present disclosure can be selected in accordance with the disease to be treated/prevented. For example, the promoter may drive expression in cells/tissue/an organ affected by the disease (i.e. cells/tissue/an organ in which the symptoms of the disease manifest).

In some embodiments, a promoter is a myeloid cell-specific promoter. In some embodiments, a promoter is a macrophage-specific promoter (e.g. the CD68 promoter).

A sequence for controlling expression of the nucleic acid may provide for expression of the nucleic acid in response to e.g. an agent/signal. For example, expression may be under the control of inducible promoter. The agent may provide for inducible expression of the nucleic acid in vivo by administration of the agent to a subject having been administered with a modified cell according to the disclosure, or ex vivo/in vitro by administration of the agent to cells in culture ex vivo or in vitro.

In some embodiments the nucleic acid(s)/vector(s) employ a conditional expression system for controlling expression of the nucleic acid encoding a inflammasome inhibitor by cells comprising the nucleic acid(s)/vector(s). ‘Conditional expression’ may also be referred to herein as ‘inducible expression’, and refers to expression contingent on certain conditions, e.g. the presence of a particular agent. Conditional expression systems are well known in the art and are reviewed e.g. in Ryding et al. Journal of Endocrinology (2001) 171, 1-14, which is hereby incorporated by reference in its entirety.

### Cells Comprising/Expressing the Polypeptides of the Disclosure

The present disclosure also provides a cell comprising or expressing a polypeptide according to the present disclosure. Also provided is a cell comprising or expressing a nucleic acid or a vector according to the present disclosure. The cell comprising or expressing a polypeptide, nucleic acid or vector according to the present disclosure may secrete a polypeptide according to the present disclosure. That is, expression of the polypeptide, nucleic acid or vector may result in the soluble production of a polypeptide according of the present disclosure from the cell.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell is, or is derived from, a cell type commonly used for the expression of polypeptides for use in therapy in humans. Exemplary cells are described e.g. in Kunert and Reinhart, Appl Microbiol Biotechnol. (2016) 100:3451-3461 (hereby incorporated by reference in its entirety), and include e.g. CHO, HEK 293, PER.C6, NS0 and BHK cells.

The present disclosure also provides a method for producing a cell comprising a nucleic acid(s) or vector(s) according to the present disclosure, comprising introducing a nucleic acid or a vector according to the present disclosure into a cell. In some embodiments, introducing an isolated nucleic acid(s) or vector(s) according to the present disclosure into a cell comprises transformation, transfection, electroporation or transduction (e.g. retroviral transduction).

The present disclosure also provides a method for producing a cell expressing/comprising a polypeptide according to the present disclosure, comprising introducing a nucleic acid or a vector according to the present disclosure in a cell. In some embodiments, the methods additionally comprise culturing the cell under conditions suitable for expression of the nucleic acid(s) or vector(s) by the cell. In some embodiments, the methods are performed in vitro.

The present disclosure also provides cells obtained or obtainable by the methods according to the present disclosure.

### Producing the Polypeptides of the Disclosure

Polypeptides according to the present disclosure may be prepared according to methods for the production of polypeptides known to the skilled person.

Polypeptides may be prepared by chemical synthesis, e.g. liquid or solid phase synthesis. For example, peptides/polypeptides can by synthesised using the methods described in, for example, Chandrudu et al., Molecules (2013), 18:4373-4388, which is hereby incorporated by reference in its entirety.

Alternatively, polypeptides may be produced by recombinant expression. Molecular biology techniques suitable for recombinant production of polypeptides are well known in the art, such as those set out in Green and Sambrook, Molecular Cloning: A Laboratory Manual (4th Edition), Cold Spring Harbor Press, 2012, and in Nat Methods. (2008); 5 (2): 135-146 both of which are hereby incorporated by reference in their entirety.

For recombinant production according to the present disclosure, any cell suitable for the expression of polypeptides may be used. The cell may be a prokaryote or eukaryote. In some embodiments the cell is a prokaryotic cell, such as a cell of archaea or bacteria. In some embodiments the bacteria may be Gram-negative bacteria such as bacteria of the family Enterobacteriaceae, for example Escherichia coli. In some embodiments, the cell is a eukaryotic cell such as a yeast cell, a plant cell, insect cell or a mammalian cell, e.g. a cell described hereinabove.

In some cases, the cell is not a prokaryotic cell because some prokaryotic cells do not allow for the same folding or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

Production may involve culture or fermentation of a eukaryotic cell modified to express the polypeptide(s) of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide(s). Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the polypeptide(s) of interest may be isolated. Any suitable method for separating proteins from cells known in the art may be used. In order to isolate the polypeptide, it may be necessary to separate the cells from nutrient medium. If the polypeptide(s) are secreted from the cells, the cells may be separated by centrifugation from the culture media that contains the secreted polypeptide(s) of interest. If the polypeptide(s) of interest collect within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polypeptide(s) of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for isolating/purifying polypeptides are known in the art, for example ion exchange chromatography and size chromatography. The polypeptide may also be affinity-purified using an appropriate binding partner for a molecular tag on the polypeptide (e.g. a His, FLAG, Myc, GST, MBP, HA, E, or Biotin tag). These techniques may be used as an alternative to precipitation, or may be performed subsequently to precipitation. In some cases it may further be desired to process the polypeptide, e.g. to remove a sequence of amino acids, molecular tag, moiety, etc.

In some embodiments, treatment is with an appropriate endopeptidase for the cleavage and removal of an amino acid sequence. In some embodiments, treatment is with an enzyme to remove the moiety of interest. In some embodiments, the polypeptide is treated to remove glycans (i.e. the polypeptide is degylcosylated), e.g. by treatment with a glycosidase such as with a Peptide: N-glycosidase (PNGase).

Other methods for distinguishing different proteins are known in the art, for example ion exchange chromatography and size chromatography. These may be used as an alternative to precipitation or may be performed subsequently to precipitation.

Once the polypeptide(s) of interest have been isolated from culture it may be desired or necessary to concentrate the polypeptide(s). A number of methods for concentrating proteins are known in the art, such as ultrafiltration or lyophilisation.

In some embodiments, the production of the polypeptide occurs in vivo, e.g. after introduction to the host of a cell comprising a nucleic acid or vector encoding a polypeptide of the present disclosure, or following introduction into a cell of the host of a nucleic acid or vector encoding a polypeptide of the present disclosure. In such embodiments, the polypeptide is transcribed, translated and post-translationally processed to the mature polypeptide. In some embodiments, the polypeptide is produced in situ at the desired location in the host.

### Compositions

The present disclosure also provides compositions comprising the polypeptides, nucleic acids, expression vectors and cells described herein.

The polypeptides, nucleic acids, expression vectors and cells described herein may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion.

Suitable formulations may comprise a polypeptide in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments the composition is formulated for injection or infusion, e.g. into a blood vessel or tissue/organ of interest.

Compositions according to the present disclosure comprise a polypeptide according to the disclosure and an agent for facilitating delivery to and/or uptake by a cell/tissue, e.g. a cell type of interest to modify (e.g. a macrophage). Strategies for facilitating intracellular delivery of molecular cargo are reviewed e.g. in Li et al., Int. J. Mol. Sci. (2015) 16:19518-19536 and Fu et al., Bioconjug Chem. (2014) 25 (9): 1602-1608, which are hereby incorporated by reference in their entirety.

In some embodiments, a composition comprises a polypeptide according to the disclosure formulated with a cationic polymer. In some embodiments, a composition comprises a polypeptide according to the disclosure encapsulated in, or immobilised on, a nanoparticle, liposome, nanogel or nanolipogel.

Nanoparticles are reviewed e.g. in Mitchell et al., Nature Reviews Drug Discovery (2021) 20:101-124, which is hereby incorporated by reference in its entirety. In some embodiments, a nanoparticle is a polymeric nanoparticle (e.g. a polymersome, dendrimer, polymer micelle, nanogel or nanosphere), an inorganic nanoparticle (e.g. a silica nanoparticle, quantum dot, iron oxide nanoparticle or gold nanoparticle), or a lipid-based nanoparticle (e.g. a liposome, lipid nanoparticle or emulsion).

Nanolipogels are described e.g. in Cao et al., Nanoscale Adv. (2020) 2:1040-1045, which is hereby incorporated by reference in its entirety. In some embodiments, a nanolipogel is a nano-sized core-shell system with a gelled core and a lipid bilayer.

In some embodiments a polypeptide or nucleic acid described herein is (covalently or non-covalently) associated with a cell-penetrating peptide (e.g. a cell-penetrating peptide described hereinabove), a cationic polymer, a cationic lipid or a viral carrier. In some embodiments a polypeptide or nucleic acid described herein is associated with a peptide/polypeptide (e.g. antibody, peptide aptamer, ligand for a cell surface molecule/fragment thereof) or a nucleic acid (e.g. nucleic acid aptamer) capable of binding to a target cell of interest (e.g. a macrophage) or an antigen thereof (a cell surface molecule expressed by a macrophage).

The present disclosure also provides methods for the production of pharmaceutically useful compositions, such methods of production may comprise one or more steps selected from: producing a polypeptide, nucleic acid, expression vector or cell described herein; isolating a polypeptide, nucleic acid, expression vector or cell described herein; and/or mixing a polypeptide, nucleic acid, expression vector or cell described herein with a pharmaceutically acceptable carrier, adjuvant, excipient or diluent.

For example, a further aspect the present disclosure provides a method of formulating or producing a medicament or pharmaceutical composition for use in the treatment of a disease/condition (e.g. a disease described hereinbelow), the method comprising formulating a pharmaceutical composition or medicament by mixing a polypeptide, nucleic acid, expression vector or cell described herein with a pharmaceutically acceptable carrier, adjuvant, excipient or diluent.

In aspects and embodiments of the present disclosure, a polypeptide may be provided in a composition comprising particular chemical constituents in specified concentrations/proportions.

In some embodiments, a polypeptide is provided in a buffer. As used herein, a “buffer” refers to a buffered solution that resists changes in pH by the action of its acid-base conjugate components. A buffer of the present disclosure preferably has a pH in the range from about 4.5 to about 7.0, preferably from about 5.0 to about 6.5. Examples of buffers that will control the pH in this range include acetate, histidine, histidine-arginine, histidine-methionine and other organic acid buffers.

### Methods for Identifying ASC Inhibitors

The present disclosure also provides methods for identifying ASC inhibitors.

Methods for identifying ASC inhibitors generally comprise analysing a putative ASC inhibitor in order to determine whether it reduces/inhibits ASC expression and/or function. In some embodiments, the reduction/inhibition is determined relative to the level of expression and/or the level of the relevant function observed for an appropriate negative control condition. In some embodiments, reduction/inhibition of ASC expression and/or function is determined by reference to the level of expression/the relevant function observed in the absence of the putative ASC inhibitor. In some embodiments, reduction/inhibition of ASC expression and/or function is determined by reference to the level of expression/the relevant function observed in the presence of an equivalent quantity of an agent known not to reduce or inhibit ASC expression or function. Where a putative ASC inhibitor is determined by such analysis to reduce/inhibit ASC expression and/or function, the putative ASC inhibitor is identified as being an ASC inhibitor.

A given agent may be evaluated for the ability to reduce or inhibit ASC expression or function using suitable assays as described herein, e.g. an assay described in Example 7. In some embodiments, the assays may be e.g. in vitro assays, optionally cell-based assays or cell-free assays. In some embodiments, the assays may be e.g. in vivo assays, i.e. performed in non-human animals.

In some embodiments, the methods are for identifying an ASC inhibitor capable of reducing/inhibiting interaction between ASC and an interaction partner for ASC (e.g. an interaction partner for ASC as described hereinabove).

Accordingly, in some embodiments, the methods for identifying ASC inhibitors according to the present disclosure comprise analysing a putative ASC inhibitor in order to determine if it reduces or inhibits interaction between ASC and an interaction partner for ASC. In some embodiments, reduction/inhibition is determined relative to the level of interaction between ASC and an interaction partner for ASC observed for an appropriate negative control condition. In some embodiments, reduction/inhibition of interaction between ASC and an interaction partner for ASC is determined by reference to the level of interaction observed in the absence of the putative ASC inhibitor. In some embodiments, reduction/inhibition of interaction between ASC and an interaction partner for ASC is determined by reference to the level of interaction observed in the presence of an equivalent quantity of an agent known not to reduce or inhibit interaction between ASC and the interaction partner for ASC. Where a putative ASC inhibitor is determined by such analysis to reduce/inhibit interaction between ASC and an interaction partner for ASC, the putative ASC inhibitor is identified as being an ASC inhibitor.

The ability of a putative ASC inhibitor to inhibit interaction between ASC and an interaction partner for ASC can be evaluated for example by analysis of the level of interaction in the presence of, or following incubation of one or both of the interaction partners with, the putative ASC inhibitor. A putative ASC inhibitor which is capable of inhibiting a given interaction (e.g. between ASC and an ASC-interaction partner) is identified by the observation of a reduction/decrease in the level of interaction between the interaction partners in the presence of—or following incubation of one or both of the interaction partners with—the putative ASC inhibitor, as compared to the level of interaction observed in the absence of the putative ASC inhibitor (or in the presence of an appropriate control agent).

Aspects and embodiments of methods for identifying ASC inhibitors in accordance with the present disclosure comprise analysis using a reporter system. The reporter system comprises: (i) an ASC polypeptide or a domain thereof, and (ii) an interaction partner for the ASC polypeptide or a domain thereof of (i), wherein one or both of (i) and (ii) further comprise a moiety/moieties providing for the production of a detectable signal upon interaction between (i) and (ii).

In some embodiments, (i) and (ii) are linked to reporter components, which upon interaction between (i) and (ii) cooperate to produce a detectable signal. The reporter components may produce a detectable signal contingent on their being brought into close physical proximity with one another.

In some embodiments, (i) and/or (ii) may comprise a PYD domain. A PYD domain may comprise an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:4.

In some embodiments, (i) and/or (ii) may comprise a CARD domain. A CARD domain may comprise an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:5.

In some embodiments, the ASC polypeptide or a domain thereof of (i) comprises ASC (i.e. ASC as described hereinabove), or a fragment thereof. A fragment of ASC may comprise one or more domains of ASC (e.g. PYD and/or CARD).

In some embodiments, the ASC polypeptide or a domain thereof of (i) comprises the PYD domain of ASC. In some embodiments, ASC polypeptide or a domain thereof of (i) comprises an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:4.

In some embodiments, the ASC polypeptide or a domain thereof of (i) comprises the CARD domain of ASC. In some embodiments, ASC polypeptide or a domain thereof of (i) comprises an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:5.

In some embodiments, the interaction partner for the ASC polypeptide or a domain thereof of (i) comprises an interaction partner for ASC (i.e. an interaction partner for ASC as described hereinabove), or a fragment thereof. A fragment of an interaction partner for ASC may comprise one or more domains of an interaction partner for ASC. It will be appreciated that the interaction partner for the ASC polypeptide or a domain thereof of (i) must be capable of interacting with the ASC polypeptide or a domain thereof of (i).

In some embodiments, the interaction partner for the ASC polypeptide or a domain thereof of (i) comprises the PYD domain of ASC. In some embodiments, the interaction partner for the ASC polypeptide or a domain thereof of (i) comprises an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO: 4.

In some embodiments, the interaction partner for the ASC polypeptide or a domain thereof of (i) comprises the CARD domain of ASC. In some embodiments, the interaction partner for the ASC polypeptide or a domain thereof of (i) comprises an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO: 5.

In some embodiments, the ASC-derived polypeptide of (i) comprises the PYD domain of ASC, and the interaction partner for the ASC polypeptide or a domain thereof of (i) comprises the PYD domain of ASC. In some embodiments, the ASC-derived polypeptide of (i) comprises an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:4, and the interaction partner for the ASC polypeptide or a domain thereof of (i) comprises an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:4.

In some embodiments, the ASC-derived polypeptide of (i) comprises the CARD domain of ASC, and the interaction partner for the ASC polypeptide or a domain thereof of (i) comprises the CARD domain of ASC. In some embodiments, the ASC-derived polypeptide of (i) comprises an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:5, and the interaction partner for the ASC polypeptide or a domain thereof of (i) comprises an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:5.

The reporter components of (i) and (ii) may be components of a suitable pair for producing a detectable signal upon being brought into close physical proximity. Suitable such reporter pairs are well known to the skilled person, and are reviewed e.g. in Blaszczak et al., Biochemical Society Transactions (2021) 49:1337-1348 and Xing et al., Plant Physiol (2016): 171 (2): 727-758, both of which are hereby incorporated by reference in their entirety.

In some embodiments, the reporter components of (i) and (ii) are components of a protein-fragment complementation system. Protein-fragment complementation systems produce a detectable signal upon physical interaction between the constituent fragments. Protein-fragment complementation systems include e.g. yeast-two hybrid, split ubiquitin, split luciferase, biomolecular fluorescence complementation (BiFC; e.g. split Venus), and bimolecular luciferase complementation (BiLC; e.g. NanoLuc Binary Technology (NanoBiT) systems. BiFC and split Venus systems are described e.g. in Kodama et al., Biotechniques (2010) 49 (5): 793-805, Ohashi et al., BioTechniques (2012) 52:45-50 and Shyu et al., Biotechniques (2006) 40 (1): 61-6, all of which are hereby incorporated by reference. NanoLuc and NanoBiT are described e.g. in Thirukkumaran et al., Front Chem. (2020) 7:938 and Dixon et al., ACS Chem. Biol. (2016) 11 (2): 400-408, both of which are hereby incorporated by reference.

In some embodiments, the reporter components of (i) and (ii) are components of a resonance energy transfer system. Resonance energy transfer systems include e.g. FRET and BRET (e.g. NanoBRET) systems, which are described e.g. in Ciruela, Curr Opin Biotechnol. (2008) 19 (4): 338-43, which is hereby incorporated by reference. NanoBRET is described e.g. in Machleidt et al. ACS Chem. Biol. (2015) 10:1797-1804, which is hereby incorporated by reference.

In some embodiments, the reporter components of (i) and (ii) are components of a protein-fragment complementation system selected from: a yeast-two hybrid system, a split ubiquitin system, a split luciferase system, a BiFC system, a split Venus system, a BiLC system and a NanoBIT system. In some embodiments, the reporter components of (i) and (ii) are components of a protein-fragment complementation system selected from: a BiFC system, a split Venus system, a BiLC system, a NanoBIT system, and a NanoBRET system.

The reporter components of a NanoBiT system comprise luciferase split into two complementary subunits: Large BIT (LgBIT) and Small BiT (SmBIT). The LgBiT subunit may be around 18 kDa, while the SmBIT may be a short complementary peptide of around 11 amino acids. A LgBiT subunit may comprise or consist of the amino acid sequence of SEQ ID NO:20. A SmBIT subunit may comprise or consist of the amino acid sequence of SEQ ID NO:19. Interaction of (i) and (ii) results the formation of a complex between LgBiT and SmBIT and the reassembly of a functional luciferase. The functional luciferase can generate a luminescent signal in the presence of substrate, e.g. furimazine.

In some embodiments, the reporter components of (i) and (ii) are components of a NanoBIT system. In some embodiments, (i) comprises a LgBiT moiety, and (ii) comprises a SmBiT moiety. In some embodiments, (i) comprises a SmBIT moiety, and (ii) comprises a LgBiT moiety. In some embodiments, (i) comprises the amino acid sequence of SEQ ID NO:20, and (ii) comprises the amino acid sequence of SEQ ID NO:19. In some embodiments, (i) comprises the amino acid sequence of SEQ ID NO:19, and (ii) comprises the amino acid sequence of SEQ ID NO:20.

The reporter components of a BiFC system (e.g. split Venus system) comprise a fluorescent protein split into two complementary fragments. Generally, fluorescent proteins consist of 11 antiparallel β-strands forming a β-barrel, with an α-helix inside and several short helical structures. The chromophore, located in the α-helix within the β-barrel structure, is chemically formed by three residues. In a BiFC system the fluorescent protein is split at a loop or within a β-stand. The two resulting non-fluorescent fragments can then be fused to proteins of interest. Interaction of the target proteins will bring the fragments within proximity, allowing the reporter protein to reform in its native three-dimensional structure and emit its fluorescent signal.

Many different fluorescent proteins can be used in BiFC systems e.g. GFP, EGFP, YFP, EYFP, CFP, ECFP, Venus, Citrine, Cerulean, mRFP1-Q66T, mCherry, mLumin, mNeptune, mKG, Dronpa, iRFP.

In the split Venus system the fluorescent protein is Venus, a modified yellow fluorescent protein derived from Aequorea Victoria. Several combinations of fluorescent protein N- and C-terminal fragments support biomolecular fluorescence complementation. Fragments of Venus may be designated VNx and VCy, which correspond to the amino acid sequences of 1-x and y-238, respectively. For example, VN173 comprises amino acids 1-173 of Venus, and VC155 comprises 155-238 of Venus.

Mutations can also be introduced into the fluorescent reporter proteins which reduce background fluorescence produced by spontaneous self-assembly of the fluorescent reporter protein, e.g. as described in Kodama & Hu, BioTechniques (2018) 53 (5) 285-298, which is hereby incorporated by reference.

In some embodiments, the reporter components of (i) and (ii) are components of a BiFC system, e.g. a split Venus system. In some embodiments, (i) comprises a VN moiety, and (ii) comprises a VC moiety. In some embodiments, (i) comprises a VC moiety, and (ii) comprises a VN moiety. In some embodiments, (i) comprises a VN173 moiety, and (ii) comprises a VC155 moiety. In some embodiments, (i) comprises a VC155 moiety, and (ii) comprises a VN173 moiety.

In some embodiments, the reporter system comprises: (i) an ASC polypeptide or a domain thereof linked to a SmBiT moiety, and (ii) an interaction partner for the ASC polypeptide or a domain thereof of (i) linked to a LgBiT moiety. In some embodiments, the reporter system comprises: (i) an ASC polypeptide or a domain thereof linked to a LgBiT moiety, and (ii) an interaction partner for the ASC polypeptide or a domain thereof of (i) linked to a SmBIT moiety.

In some embodiments, the reporter system comprises: (i) the PYD domain of ASC linked to a SmBIT moiety, and (ii) the PYD domain of ASC linked to a LgBiT moiety.

In some embodiments, the term “close physical proximity” means a physical interaction between the reporter components of (i) and (ii), e.g. the formation of a protein-protein complex. A physical interaction may be the formation of a non-covalent complex formed of the reporter components of (i) and (ii), e.g. via electrostatic interaction (e.g. ionic bonding, hydrogen bonding) and/or Van der Waals forces.

As used herein, the term “close physical proximity” may mean a physical proximity such that energy may be transferred between reporter components of (i) and (ii). For example, in FRET/BRET based systems close physical proximity may mean less than 100 angstroms, e.g. less than 90, less than 80, less than 70, less than 60, less than 50, less than 40, less than 30, less than 20 or less than 10 angstroms. In some FRET/BRET based systems close physical proximity may mean between 10 and 100 angstroms, e.g. between 30 and 60 angstroms.

As used herein, a “reporter signal” is a detectable signal/activity produced by the reporter components upon interaction of (i) and (ii).

The detectable signal/activity may be visual. For example, the detectable signal/activity may be electromagnetic emission e.g. detectable light, fluorescence, luminescence. A detectable signal/activity may be electromagnetic emission of a given wavelength.

The detectable signal/activity may be an enzymatic activity e.g. luciferase, β-galactosidase, β-lactamase, antibiotic resistance. In some embodiments the reporter system is contacted with a substrate. In some embodiments the enzymatic activity results in a coloured reaction product. In some embodiments the enzymatic activity results in product which fluoresces or luminesces. In some embodiments the enzymatic activity imparts antibiotic resistance.

In some embodiments, the ASC polypeptide or domain thereof of (i) and/or the interaction partner for the ASC polypeptide or domain thereof of (i) are linked/connected/conjugated to reporter components.

Reporter components may be covalently or non-covalently linked to the ASC polypeptide or domain thereof of (i) and/or the interaction partner for the ASC polypeptide or domain thereof of (i). In some embodiments the reporter component is covalently linked to the ASC polypeptide or domain thereof of (i) and/or the interaction partner for the ASC polypeptide or domain thereof of (i). For example, the reporter component and the ASC polypeptide or domain thereof of (i) and/or the interaction partner for the ASC polypeptide or domain thereof of (i) may be linked by a peptide bond. In some embodiments the reporter construct and the ASC polypeptide or domain thereof of (i) and/or the interaction partner for the ASC polypeptide or domain thereof of (i) are linked by a linker sequence. In some embodiments the reporter construct and the ASC polypeptide or domain thereof of (i) and/or the interaction partner for the ASC polypeptide or domain thereof of (i) are expressed as a fusion protein.

Linker sequences are known to the skilled person, and are described, for example in Chen et al., Adv Drug Deliv Rev (2013) 65 (10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65 (10): 1357-1369. Flexible linker sequences often comprise high proportions of glycine and/or serine residues.

In some embodiments, the linker sequence comprises at least one glycine residue and/or at least one serine residue. In some embodiments the linker sequence consists of glycine and serine residues. In some embodiments, the linker sequence has a length of 1-2, 1-3, 1-4, 1-5, 1-10, 1-15, 1-20, 1-30 or 1-40 amino acids.

Alternatively, the reporter component may be connected to the polypeptide of interest by a hydrocarbon linker e.g. a polyethylene glycol (PEG) linker. PEG linkers may comprise one or more hexaethyleneglycol residues joined by phosphodiester bonds.

As used herein, “contacting” may refer to bringing together, co-expressing, incubating or mixing the reporter system and the putative ASC inhibitor.

In some embodiments, contacting/incubating/mixing an agent with a reporter system comprises expressing said agent and reporter system in the same cell. In some embodiments, contacting/incubating/mixing an agent with a reporter system comprises treating cells which express reporter system components with said agent. In some embodiments, “contacting” an agent with a reporter system comprises contacting/incubating/mixing samples comprising an agent and a reporter e.g. in an appropriate container or on an appropriate solid support.

As used herein, a “putative ASC inhibitor” refers to any agent which may be capable of inhibiting, or is predicted to inhibit, ASC expression and/or function. In some embodiments a putative ASC inhibitor is capable of inhibiting ASC expression and/or function. In some embodiments a putative ASC inhibitor is not capable of inhibiting ASC expression and/or function.

In some embodiments, a “putative ASC inhibitor” refers to any agent which may be capable of reducing or inhibiting the interaction between ASC and an interaction partner for ASC. In some embodiments, a putative ASC inhibitor is capable of reducing or inhibiting the interaction between ASC and an interaction partner for ASC. In some embodiments, a putative ASC inhibitor is not capable of reducing or inhibiting the interaction between ASC and an interaction partner for ASC.

A putative ASC inhibitor may be referred to herein as an agent. Agents which may be capable of inhibiting ASC (e.g. reducing or inhibiting the interaction between ASC and an interaction partner for ASC) may be of any kind. In some embodiments the agent may be an antibody, an antigen-binding fragment thereof, a polypeptide, a peptide, a nucleic acid, an oligonucleotide, an aptamer or a small molecule, e.g. as described herein. The agents may be provided in isolated or purified form.

A putative ASC inhibitor which is found to be capable of inhibiting ASC expression and/or function (e.g. capable of reducing or inhibiting the interaction between ASC and an interaction partner for ASC) may be referred to as an ASC inhibitor.

In some embodiments, an ASC inhibitor causes a reduced or inhibited interaction between ASC and an interaction partner for ASC.

In some embodiments, the ASC inhibitor is a competitive inhibitor of the interaction between ASC and an interaction partner for ASC. In some embodiments, the ASC inhibitor is an allosteric inhibitor of the interaction between ASC and an interaction partner for ASC. In some embodiments, the ASC inhibitor may form a complex with ASC or the ASC-interaction partner. A complex with ASC/ASC-interacting partner may refer to a non-covalent complex comprising ASC/ASC-interacting partner and the ASC inhibitor, formed e.g. via electrostatic interaction (e.g. ionic bonding, hydrogen bonding) and/or Van der Waals forces.

In some embodiments, the ASC inhibitor binds to ASC in a region required for interaction with an interacting partner for ASC. In some embodiments, the ASC inhibitor binds to the PYD-domain of ASC. In some embodiments, the ASC inhibitor binds to the CARD-domain of ASC.

In some embodiments, the ASC inhibitor causes a reduced or inhibited interaction between PYD domains. In some embodiments, the ASC inhibitor causes a reduced or inhibited interaction between CARD domains.

In some embodiments, the ASC inhibitor causes a reduced or inhibited interaction between at least one (e.g. one, two or three) interfaces involved in ASC-PYD domain homotypic interactions. In some embodiments, the ASC inhibitor causes a reduced or inhibited interaction between at least one (e.g. one, two or three) interfaces involved in ASC-CARD domain homotypic interactions.

As used herein, “reduced” or “inhibited” interaction may be a level of interaction which is less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of interaction between ASC and an ASC-interacting partner observed in the negative control condition.

Alternatively, “reduced” or “inhibited” interaction may be expressed in terms of percentage inhibition of the level of interaction between ASC and the ASC-interaction partner observed in the negative control condition. In such instances, “reduced” or “inhibited” interaction may refer to percentage inhibition of greater than 0%, e.g. greater than 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, or 100% of the level of interaction between ASC and the ASC-interaction partner observed in the negative control condition.

In some embodiments, the methods comprise detecting the level of a reporter signal. The level of reporter signal may be detected by any suitable means well known to the skilled person.

In some embodiments, the methods comprise determining whether the putative ASC inhibitor causes a reduced or decreased level of the reporter signal.

In some embodiments, a reduced/decreased level of reporter signal is relative to the level of reporter signal in the absence of an ASC inhibitor (and/or in the presence of a suitable control agent known not to inhibit interaction between the ASC polypeptide/domain thereof and interaction partner therefor).

As used herein, a “reduced” or “decreased” level of reporter signal may be a level of reporter signal which is less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, $0.25 times, ≤0.2 times, ≤0.15 times, ≤0.1 times, ≤0.05 times, or ≤0.01 times the level of reporter signal observed in the negative control condition.

Alternatively, a “reduced” or “decreased” level of reporter signal may be expressed in terms of percentage inhibition of the level of reporter signal observed in the negative control condition. In such instances, a “reduced” or “decreased” level of reporter signal may refer to percentage inhibition of greater than 0%, e.g. greater than 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, or 100% of the level of reporter signal observed in the negative control condition.

An appropriate negative control condition may e.g. employ an agent known to lack the ability to reduce or inhibit the level of interaction between ASC and an ASC-interaction partner or an agent that is not capable of inhibiting ASC expression and/or function.

In some embodiments a background level of reporter signal is determined. The background level of reporter signal may be determined by detecting the level of reporter signal wherein one of the reporter components is linked to a mutant ASC or domain thereof. In some embodiments the mutant ASC or domain thereof comprises one or more mutations (e.g. one, two or three) in the PYD domain (e.g. E13R, K21E/K22E/, F59E). In some embodiments the mutant ASC or domain thereof comprises one or more (e.g. one, two or three) mutations in the CARD domain (e.g. R125E, D134R, R160E). In some embodiments the mutant ASC comprises one or more mutations (e.g. one, two or three) in each of the PYD and CARD domain.

In some embodiments, an ASC polypeptide or a domain thereof linked to a first reporter component, and/or an ASC interacting partner polypeptide or a domain thereof linked to a second reporter component are encoded by a nucleotide sequence. In some embodiments the putative ASC inhibitor is encoded by a nucleotide sequence. In some embodiments (i), (ii) and the putative ASC inhibitor are encoded by a nucleotide sequence. In such embodiments, the methods may comprise a step of introducing (e.g. by transfection) into cells a nucleotide sequence(s).

### Kits

In some aspects of the present disclosure a kit of parts is provided. In some embodiments the kit may have at least one container having a predetermined quantity of a polypeptide, nucleic acid, expression vector, cell or composition described herein.

In some embodiments, the kit may comprise materials for producing a polypeptide, nucleic acid, expression vector, cell or composition described herein. For example, the kit may comprise materials for modifying a cell to express or comprise a polypeptide, nucleic acid, expression vector, according to the present disclosure, or materials for introducing into a cell the nucleic acid, expression vector, according to the present disclosure.

The kit may provide a polypeptide, nucleic acid, expression vector, cell or composition together with instructions for administration to a patient in order to treat or prevent a specified disease/condition, e.g. a disease/condition described hereinbelow.

In some embodiments the kit may further comprise at least one container having a predetermined quantity of another therapeutic/prophylactic agent (e.g. a therapeutic/prophylactic agent for the treatment/prevention of a disease/condition described herein). In such embodiments, the kit may also comprise a second medicament or pharmaceutical composition such that the two medicaments or pharmaceutical compositions may be administered simultaneously or separately such that they provide a combined treatment/prevention for the specific disease or condition.

The present disclosure also provides kits for identifying an ASC inhibitor. Kits of the present disclosure may comprise a reporter system described herein, or a part thereof. The reporter system comprises: (i) an ASC polypeptide or a domain thereof, and (ii) an interaction partner for the ASC polypeptide or a domain thereof of (i), wherein one or both of (i) and (ii) further comprise a moiety/moieties providing for the production of a detectable signal upon interaction between (i) and (ii). The kit may comprise any one or more components of the reporter system.

In some embodiments the kit additionally comprises a substrate, e.g. the substrate for an enzyme of the reporter system.

In some embodiments the kit additionally comprises means for detecting the detectable signal. Suitable means for such detection can be selected by the skilled person in accordance with the entity/signal to be detected.

In some embodiments the kit comprises nucleic acid(s) encoding the polypeptides (i) and/or (ii) described herein.

In some embodiments the kit comprises a cell comprising the nucleic acid(s) encoding the polypeptides (i) and/or (ii) described herein.

Kits according to the present disclosure may include instructions for use, e.g. in the form of an instruction booklet or leaflet. The instructions may include a protocol for performing any one or more of the methods described herein.

### Further Articles of the Disclosure

The present disclosure also provides the constituent polypeptides of the kits and methods described herein. The polypeptides may be provided in isolated or substantially purified form.

In some embodiments, the polypeptide may comprise a reporter component (i.e. a reporter component as described hereinabove).

In some embodiments, the polypeptide may comprise a reporter component of a NanoBiT system, e.g. selected from a LgBIT and a SmBIT.

In some embodiments, the polypeptide may comprise a reporter component of a BiFC system. In some embodiments, the polypeptide may comprise a fragment of a fluorescent protein, e.g. a fragment of GFP, EGFP, YFP, EYFP, CFP, ECFP, Venus, Citrine, Cerulean, mRFP1-Q66T, mCherry, mLumin, mNeptune, mKG, Dronpa, or iRFP. In some embodiments, the polypeptide may comprise a fragment of Venus, e.g. selected from amino acids 1-173 of Venus and acids 155-238 of Venus.

In some embodiments, the polypeptide may comprise a PYD domain. A PYD domain may comprise an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:4. In some embodiments, the polypeptide may further comprise a reporter component (i.e. a reporter component as described hereinabove).

In some embodiments, the polypeptide may comprise a CARD domain. A CARD domain may comprise an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:5. In some embodiments, the polypeptide may further comprise a reporter component (i.e. a reporter component as described hereinabove).

In some embodiments, the polypeptide comprises ASC (i.e. ASC as described hereinabove), or a fragment thereof. A fragment of ASC may comprise one or more domains of ASC (e.g. PYD and/or CARD). In some embodiments, the polypeptide comprises the PYD domain of ASC. In some embodiments the polypeptide comprises an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:4. In some embodiments, the polypeptide comprises the CARD domain of ASC. In some embodiments, the polypeptide comprises an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:5. In some embodiments, the polypeptide may further comprise a reporter component (i.e. a reporter component as described hereinabove).

In some embodiments, the polypeptide comprises an interaction partner for ASC (i.e. an interaction partner for ASC as described hereinabove), or a fragment thereof. A fragment of an interaction partner for ASC may comprise one or more domains of an interaction partner for ASC. In some embodiments, the polypeptide comprises the PYD domain of ASC. In some embodiments, the polypeptide comprises an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:4. In some embodiments, the polypeptide comprises the CARD domain of ASC. In some embodiments, the polypeptide comprises an amino acid sequence having at least 50% sequence identity, preferably one of 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of SEQ ID NO:5. In some embodiments, the polypeptide may further comprise a reporter component (i.e. a reporter component as described hereinabove).

Polypeptides according to the invention may be prepared according to methods for the production of polypeptides known to the skilled person.

Polypeptides may be prepared by chemical synthesis, e.g. liquid or solid phase synthesis. For example, peptides/polypeptides can by synthesised using the methods described in, for example, Chandrudu et al., Molecules (2013), 18:4373-4388, which is hereby incorporated by reference in its entirety.

Alternatively, polypeptides may be produced by recombinant expression. Molecular biology techniques suitable for recombinant production of polypeptides are well known in the art, such as those set out in Green and Sambrook, Molecular Cloning: A Laboratory Manual (4th Edition), Cold Spring Harbor Press, 2012, and in Nat Methods. (2008); 5 (2): 135-146 both of which are hereby incorporated by reference in their entirety.

For recombinant production according to the invention, any cell suitable for the expression of polypeptides may be used. The cell may be a prokaryote or eukaryote. In some embodiments the cell is a prokaryotic cell, such as a cell of archaea or bacteria. In some embodiments the bacteria may be Gram-negative bacteria such as bacteria of the family Enterobacteriaceae, for example Escherichia coli. In some embodiments, the cell is a eukaryotic cell such as a yeast cell, a plant cell, insect cell or a mammalian cell, e.g. CHO, HEK (e.g. HEK293), HeLa or COS cells. In some embodiments, the cell is a CHO cell that transiently or stably expresses the polypeptides.

In some cases, the cell is not a prokaryotic cell because some prokaryotic cells do not allow for the same folding or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

In some embodiments polypeptides may be prepared by cell-free-protein synthesis (CFPS), e.g. using a system described in Zemella et al. Chembiochem (2015) 16 (17): 2420-2431, which is hereby incorporated by reference in its entirety.

Production may involve culture or fermentation of a eukaryotic cell modified to express the polypeptide of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide. Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the polypeptide, the polypeptide of interest may be isolated. Any suitable method for separating proteins from cells known in the art may be used. In order to isolate the polypeptide it may be necessary to separate the cells from nutrient medium. If the polypeptide is secreted from the cells, the cells may be separated by centrifugation from the culture media that contains the secreted polypeptide of interest. If the polypeptide of interest collects within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polypeptide of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography. These may be used as an alternative to precipitation, or may be performed subsequently to precipitation.

Once the polypeptide of interest has been isolated from culture it may be desired or necessary to concentrate the polypeptide. A number of methods for concentrating proteins are known in the art, such as ultrafiltration or lyophilisation.

The present invention provides a nucleic acid encoding a polypeptide according to the present disclosure. It will be appreciated that “a nucleic acid” encompasses a plurality of such nucleic acids.

In some embodiments, the nucleic acid is purified or isolated, e.g. from other nucleic acid, or naturally-occurring biological material. In some embodiments the nucleic acid(s) comprise or consist of DNA and/or RNA.

The present disclosure also provides a vector comprising the nucleic acid according to the present disclosure.

The nucleotide sequence may be contained in a vector, e.g. an expression vector. A “vector” as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector according to the invention.

The term “operably linked” may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. The resulting transcript(s) may then be translated into a desired peptide(s)/polypeptide(s).

Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes).

In some embodiments, the vector may be a eukaryotic vector, e.g. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

The present disclosure also provides a cell comprising or expressing a polypeptide according to the present disclosure. Also provided is a cell comprising or expressing a nucleic acid or vector according to the present disclosure. The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

The present disclosure also provides a method for producing a cell comprising a nucleic acid or vector according to the present disclosure, comprising introducing a nucleic acid or vector according to the present disclosure into a cell. In some embodiments, introducing an isolated nucleic acid or vector according to the present disclosure into a cell comprises transformation, transfection, electroporation or transduction (e.g. retroviral transduction).

The present disclosure also provides a method for producing a cell expressing/comprising a polypeptide according to the present disclosure, comprising introducing a nucleic acid or vector according to the present invention in a cell. In some embodiments, the methods additionally comprise culturing the cell under conditions suitable for expression of the nucleic acid or vector by the cell. In some embodiments, the methods are performed in vitro.

The present invention also provides cells obtained or obtainable by the methods according to the present disclosure.

### Treatment/Prevention of Diseases and Conditions Through Inflammasome Inhibition

The present disclosure provides methods and articles (agents and compositions) for the treatment and/or prevention of diseases through inflammasome inhibition. Treatment/prevention of disease is achieved by inflammasome inhibition in e.g. a cell, tissue/organ/organ system/subject.

Aspects of the present disclosure are concerned with the treatment/prevention of diseases in which inflammasome activity is pathologically-implicated. A disease/condition in which inflammasome activity is ‘pathologically-implicated’ is a disease/condition in which inflammasome activity (e.g. as a consequence of activation of an inflammasome) is positively-associated with the disease or condition.

It will be appreciated that the inflammasome whose activity is pathologically-implicated in a disease/condition to be treated/prevented in accordance with the present disclosure may optionally be further defined in accordance with any embodiment of an inflammasome described herein.

A disease/condition in which inflammasome activity is ‘pathologically-implicated’ may be a disease/condition for which an increase in the level of inflammasome activity (relative to the healthy state) is positively associated with the onset, development and/or progression of the disease/condition. A disease/condition in which inflammasome activity is ‘pathologically-implicated’ may be a disease/condition for which an increase in the level of inflammasome activity (relative to the healthy state) is positively associated with the severity of one or more symptoms of the disease/condition. A disease/condition in which inflammasome activity is ‘pathologically-implicated’ may be a disease/condition for which an increase in the level of inflammasome activity (relative to the healthy state) is a risk factor for the onset, development and/or progression of the disease/condition.

As used herein, the ‘healthy state’ refers to the state in the absence of a disease/condition. An elevated level of inflammasome activity may be inferred by detection of an increase in the level of a correlate of inflammasome activity (e.g. IL-1B expression, IL-18 expression, caspase-1 activity, pyroptosis) as compared to the level in the healthy state.

It will be appreciated that the utility of the present disclosure extends to the treatment/prevention of any disease/condition that would derive therapeutic/prophylactic benefit from inflammasome inhibition.

Aspects and embodiments of the present disclosure relate to the treatment/prevention of diseases characterised by aberrant inflammasome activation and/or excessive inflammasome activity.

As used herein, ‘aberrant inflammasome activation’ refers to activation of the inflammasome where the inflammasome should not have been activated. As used herein, ‘excessive inflammasome activity’ refers to a level of inflammasome activity which is in excess of the level of non-pathological inflammasome activity (e.g. the normal level of activity required e.g. for resolution of an infection). Aberrant activation of the inflammasome and/or excessive inflammasome activity can give rise to or exacerbate inflammatory disease.

Diseases/conditions in which inflammasome activity is pathologically-implicated are described e.g. in Seok et al., Archives of Pharmacal Research (2021) and 44:16-35, Guo et al., Nat Med. (2015) 21 (7): 677-687, both of which are hereby incorporated by reference in their entirety.

Aberrant inflammasome activation and/or excessive inflammasome activity is implicated in the pathology of a number of diseases, including inflammatory diseases, chronic inflammatory diseases, neurodegenerative diseases (e.g. Alzheimer's disease, Parkinson's disease), autoimmune diseases (e.g. multiple sclerosis, rheumatoid arthritis, systemic lupus erythematosus (SLE)), autoinflammatory diseases (e.g. cryopyrin-associated periodic syndrome (CAPS), familial cold autoinflammatory syndrome (FCAS), Muckle-Wells syndrome (MWS), neonatal onset multisystemic inflammatory disease (NOMID)/chronic infantile neurologic cutaneous articular (CINCA)), cancers, cardiovascular disease (e.g. atherosclerosis, acute myocardial infarction), respiratory diseases (e.g. asthma, severe steroid-resistant asthma, chronic obstructive pulmonary disease (COPD)), diseases caused by viral infection, gout, non-alcoholic fatty liver disease (NAFLD), silicosis, skin diseases (e.g. acne, atopic dermatitis, psoriasis, vitiligo) and type 2 diabetes. Such diseases/conditions may be described as being characterised by aberrant inflammasome activation and/or excessive inflammasome activity.

In some aspects and embodiments, the disease/condition to be treated/presented in accordance with the present disclosure is a disease characterised by one or more of:
an increased level of IL-1β (e.g. in a subject, or in an organ/tissue in which symptoms of the disease/condition manifest) relative to the healthy state;
an increased level of IL-18 (e.g. in a subject, or in an organ/tissue in which symptoms of the disease/condition manifest) relative to the healthy state;
an increased level of caspase-1 activity (e.g. in a subject, or in an organ/tissue in which symptoms of the disease/condition manifest) relative to the healthy state;
an increased level of pyroptosis (e.g. in a subject, or in an organ/tissue in which symptoms of the disease/condition manifest) relative to the healthy state;
an increase in ASC specks (e.g. in a subject, or in an organ/tissue in which symptoms of the disease/condition manifest) relative to the healthy state; or
an increased level of inflammasome activity (e.g. in a subject, or in an organ/tissue in which symptoms of the disease/condition manifest) relative to the healthy state.

Treatment may be effective to reduce/delay/prevent the development or progression of the disease. Treatment may be effective to reduce/delay/prevent the worsening of one or more symptoms of the disease. Treatment may be effective to improve one or more symptoms of the disease. Treatment may be effective to reduce the severity of and/or reverse one or more symptoms of the disease. Treatment may be effective to reduce morbidity and/or mortality associated with the disease.

Prevention may refer to prevention of development of the disease, and/or prevention of worsening of the disease, e.g. prevention of progression of the disease, e.g. to a later/chronic stage.

In accordance with various aspects of the present invention, a method of treating and/or preventing a disease according to the present invention may comprise one or more of the following:
Reducing the level of gene/protein expression of ASC;
Reducing the level of activity of ASC;
Reducing inflammasome activation;
Reducing the level of a correlate of ASC activity and/or inflammasome activation;
Reducing the level of IL-1B;
Reducing the level of IL-18;
Reducing the level of caspase-1 activity;
Reducing the level of pyroptosis;
Reducing the number of ASC specks;
Reducing the level of inflammasome-mediated inflammation.

### Administration

Inhibition of ASC or administration of an article of the present disclosure (e.g. an ASC inhibitor, a polypeptide/nucleic acid/vector/cell/composition) to a subject in accordance with the present disclosure is preferably in a “therapeutically-effective” or “prophylactically-effective” amount, this being sufficient to show therapeutic/prophylactic benefit to the subject.

The actual amount administered, and rate and time-course of administration, will depend on the nature and severity of the disease to be treated/prevented, and the nature of the agent. Prescription of treatment, e.g. decisions on dosage etc., is within the responsibility of general practitioners and other medical doctors, and typically takes account of the disease/condition to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

In therapeutic applications, the articles of the present disclosure (e.g. ASC inhibitors, polypeptides/nucleic acids/vectors/cells/compositions) are preferably formulated as a medicament or pharmaceutical together with one or more other pharmaceutically acceptable ingredients well known to those skilled in the art, including, but not limited to, pharmaceutically acceptable carriers, adjuvants, excipients, diluents, fillers, buffers, preservatives, anti-oxidants, lubricants, stabilisers, solubilisers, surfactants (e.g., wetting agents), masking agents, colouring agents, flavouring agents, and sweetening agents.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, adjuvant, excipient, etc. must also be “acceptable” in the sense of being compatible with the other ingredients of the formulation.

Suitable carriers, adjuvants, excipients, etc. can be found in standard pharmaceutical texts, for example, Remington's Pharmaceutical Sciences, 20th Edition, 2000, pub. Lippincott, Williams & Wilkins; and Handbook of Pharmaceutical Excipients, 2nd edition, 1994.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulations may be prepared for topical, parenteral, systemic, intravenous, intra-arterial, intramuscular, intrathecal, intraocular, intra-conjunctival, subcutaneous, oral or transdermal routes of administration which may include injection. Injectable formulations may comprise the selected agent in a sterile or isotonic medium. The formulation and mode of administration may be selected according to the agent to be administered, and disease to be treated/prevented.

In some embodiments, articles of the present disclosure (e.g. ASC inhibitors, polypeptides) may be formulated for topical administration, in particular where the disease/condition to be treated/prevented is an inflammatory skin disease/condition, such as contact hypersensitivity, acne, atopic dermatitis, psoriasis or vitiligo.

In some embodiments, articles of the present disclosure (e.g. ASC inhibitors, polypeptides) may be formulated for subretinal administration, in particular where the disease/condition to be treated/prevented is an ocular disease/condition, such as acute glaucoma or age-related macular degeneration.

In some embodiments, articles of the present disclosure (e.g. ASC inhibitors, polypeptides) may be formulated for intranasal administration, in particular where the disease/condition to be treated/prevented is an airway disease/condition, such as severe steroid-resistant asthma, chronic obstructive pulmonary disease (COPD) or respiratory disease caused by viral infection.

In some embodiments, articles of the present disclosure (e.g. ASC inhibitors, polypeptides) may be formulated for systemic administration, in particular where the disease/condition to be treated/prevented is atherosclerosis, stroke, inflammatory disease caused by viral infection, a metabolic disease such as non-alcoholic fatty liver disease (NAFLD), or gout.

In some embodiments, an ASC inhibitor is a polypeptide. In some embodiments, a polypeptide according to the present disclosure may be administered in the form of nucleic acid/vector encoding the polypeptide. In some embodiments, a polypeptide according to the present disclosure may be administered in the form of cell expressing the polypeptide (e.g. from a nucleic acid/vector encoding the polypeptide).

Administration of a polypeptide, nucleic acid or vector according to the present disclosure preferably results in modification of a cell or cells to comprise the polypeptide/nucleic acid/vector.

Multiple doses of an article of the present disclosure (e.g. ASC inhibitors, polypeptides/nucleic acids/vectors/cells/compositions) may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

### Subjects

A subject in accordance with the present disclosure may be any animal. In some embodiments a subject may be mammalian. In some embodiments a subject may be human. In some embodiments a subject may be a non-human animal, e.g. a non-human mammal. The subject may be male or female.

The subject may be a patient. The patient may have a disease/condition described herein. A subject may have been diagnosed with a disease/condition described herein, may be suspected of having a disease/condition described herein, or may be at risk from developing a disease/condition described herein.

A subject/patient may be selected for therapy/prophylaxis in accordance with the present disclosure based on characterisation for markers of a disease/condition described herein.

A subject may be selected for therapy/prophylaxis in accordance with the present disclosure based on determination that the subject has one or more of: an increased level of IL-1β, IL-18, caspase-1 activity, pyroptosis, ASC speck formation or inflammasome activity (e.g. in the subject, or in an organ/tissue of the subject in which symptoms of the disease/condition manifest), e.g. relative to the healthy state.

### Sequence Identity

As used herein, ‘sequence identity’ refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J., Bioinformatics (2005) 21, 951-960), T-coffee (Notredame et al., J. Mol. Biol. (2000) 302, 205-217), Kalign (Lassmann and Sonnhammer, BMC Bioinformatics (2005) 6,298) and MAFFT (Katoh and Standley, Molecular Biology and Evolution (2013) 30 (4) 772-780) software. When using such software, the default parameters, e.g. for gap penalty and extension penalty, are preferably used.

Sequences

| SEQ | | |
|---|---|---|
| ID | | |
| NO: | DESCRIPTION | SEQUENCE |
| 1 | Human ASC Isoform | MGRARDAILDALENLTAEELKKFKLKLLSVPLREGYGRIPRGALLSMDALDLTDKLVS |
| | 1 (UniProt: Q9ULZ3- | FYLETYGAELTANVLRDMGLQEMAGQLQAATHQGSGAAPAGIQAPPQSAAKPGLH |
| | 1, v2) | FIDQHRAALIARVTNVEWLLDALYGKVLTDEQYQAVRAEPTNPSKMRKLFSFTPAW |
| | | NWTCKDLLLQALRESQSYLVEDLERS |
| 2 | Human ASC Isoform | MGRARDAILDALENLTAEELKKFKLKLLSVPLREGYGRIPRGALLSMDALDLTDKLVS |
| | 2 (UniProt: Q9ULZ3- | FYLETYGAELTANVLRDMGLQEMAGQLQAATHQGLHFIDQHRAALIARVTNVEWLL |
| | 2) | DALYGKVLTDEQYQAVRAEPTNPSKMRKLFSFTPAWNWTCKDLLLQALRESQSYLV |
| | | EDLERS |
| 3 | Human ASC Isoform | MGRARDAILDALENLTAEELKKFKLQAATHQGSGAAPAGIQAPPQSAAKPGLHFIDQ |
| | 3 (UniProt: Q9ULZ3- | HRAALIARVTNVEWLLDALYGKVLTDEQYQAVRAEPTNPSKMRKLFSFTPAWNWTC |
| | 3) | KDLLLQALRESQSYLVEDLERS |
| 4 | Human ASC Pyrin | MGRARDAILDALENLTAEELKKFKLKLLSVPLREGYGRIPRGALLSMDALDLTDKLVS |
| | domain | FYLETYGAELTANVLRDMGLQEMAGQLQAATHQ |
| 5 | Human ASC CARD | AAKPGLHFIDQHRAALIARVTNVEWLLDALYGKVLTDEQYQAVRAEPTNPSKMRKLF |
| | domain | SFTPAWNWTCKDLLLQALRESQSYLVEDLERS |
| 6 | Hupa4 ASC2 | MGTKREAILEVLENLTPEELKKFKMKLGTVPLREGFRRIPRGALGQLDIVDLTDKLVA |
| | | SYCEDYAAELVVAVLRDMGMLEEAARLQRAA |
| 7 | Human ASC2 | MGTKREAILKVLENLTPEELKKFKMKLGTVPLREGFERIPRGALGQLDIVDLTDKLVA |
| | (UniProt: Q8WXC3- | SYYEDYAAELVVAVLRDMRMLEEAARLQRAA |
| | 1, v1) | |
| 8 | Pan troglodytes | MGTKREAILKVLENLTPEELKKFKMKLGTVPLREGFERIPRGALGQLDIVDLTDKLVA |
| | ASC2 (UniProt: | SYYEDYAAELVVAVLRDMRMLEEAARLQRAA |
| | H2QB01) | |
| 9 | Propithecus | MRKKRDAIREALENLTADELKFKLKLGSVQLRQGFRNVPRGALGPLDAVDLTDKLVA |
| | coquereli ASC2 | FYCEDYYAAELTAVVLLDVGMQEKAARLQGAA |
| | (XM_012660211.1) | |
| 10 | Saimiri boliviensis | MGRKRDAILEVLENLTPEELKKFKMKLGTVPLHEGSRRIPRGAIGQLDIVDLTDKLVA |
| | ASC2 | FYREDYANELVVTVLLDMGMQEEAERLQRGA |
| | (XM_003930037.2) | |
| 11 | Pteropus alecto | MGKKRDAILEALENLTADELKKFKLKLGAAPLRQGFRNIPRGALGPLDAVDLTDKLVA |
| | ASC2 | FYCEDYGAELTAVVLRDIGMQEEAERLQGVA |
| | (XM_006914166.3) | |
| 12 | Rhinolophus | MGKKRDVILEVLENLDPSELKKFKLKLAVVPLRDGFRNIPRGSLVALDPVDLTDKLTA |
| | ferrumequinum | FYREDYGAEITAEVLRDMGMQEEAFRLQELQ |
| | ASC2 (UniProt: | |
| | A0A671G0P3) | |
| 13 | Rhinolophus sinicus | MGKKRDTILEVLENLDPSELKKFKLKLAAVPLRDGFRNIPRGSLGALDPVDLTDKLVA |
| | (XM_033102071) | FYREDYGAEITAAVLRDMGMQEEAFRLQEVQ |
| 14 | Hipposideros armiger | MGKKRDTILEVLENLDPSELKKFKLKLAAVSLPDGFRNIPRGALGSLDAVDLTDKIVA |
| | ASC2 | FYREDFGAKVAADVLTDMGMQEEASRLQELQ |
| | (XM_019628510.1) | |
| 15 | Eptesicus fuscus | MCRKRDAILDALESLTADELKKFKLKLGAAPLRPGFRHIPRGELEKLDAVDLTDRLVA |
| | ASC2 | FYREDYGAEVTAAVLRGMGMQEAAARLRGAA |
| | (XM_008152780.2) | |
| 16 | Pteronotus parnellii | MGKKRDAILEALENLTPEELKKFKLKLGTAPLREGFRNIPRGALGQLDAVDLTDKLVS |
| | ASC2 | FYCEDYGAELTAVVLRDMGMQEEASRLQGVS |
| | (AWGZ01168050) | |
| 17 | Phyllostomus | MGKKRDAILEALENLTPEELKKFKLKLGAAPLREGFRNIPRGALGQLDAMDLTDKLV |
| | discolor ASC2 | TFYCEDYGAELTAVVLRDMGMQEEAARLQRVE |
| | (UniProt: | |
| | A0A6J2MDV3) | |
| 18 | Human ASC mRNA | GGAGCCGACTTCCTCCTGGTCGGCGGCTGCAGCGGGGTGAGCGGCGGCAGCG |
| | (NCBI | GCCGGGGATCCTGGAGCCATGGGGCGCGCGCGCGACGCCATCCTGGATGCGC |
| | NM_013258.5) | TGGAGAACCTGACCGCCGAGGAGCTCAAGAAGTTCAAGCTGAAGCTGCTGTCG |
| | | GTGCCGCTGCGCGAGGGCTACGGGCGCATCCCGCGGGGCGCGCTGCTGTCCA |
| | | TGGACGCCTTGGACCTCACCGACAAGCTGGTCAGCTTCTACCTGGAGACCTAC |
| | | GGCGCCGAGCTCACCGCTAACGTGCTGCGCGACATGGGCCTGCAGGAGATGG |
| | | CCGGGCAGCTGCAGGCGGCCACGCACCAGGGCTCTGGAGCCGCGCCAGCTGG |
| | | GATCCAGGCCCCTCCTCAGTCGGCAGCCAAGCCAGGCCTGCACTTTATAGACC |
| | | AGCACCGGGCTGCGCTTATCGCGAGGGTCACAAACGTTGAGTGGCTGCTGGAT |
| | | GCTCTGTACGGGAAGGTCCTGACGGATGAGCAGTACCAGGCAGTGCGGGCCG |
| | | AGCCCACCAACCCAAGCAAGATGCGGAAGCTCTTCAGTTTCACACCAGCCTGGA |
| | | ACTGGACCTGCAAGGACTTGCTCCTCCAGGCCCTAAGGGAGTCCCAGTCCTAC |
| | | CTGGTGGAGGACCTGGAGCGGAGCTGAGGCTCCTTCCCAGCAACACTCCGGTC |
| | | AGCCCCTGGCAATCCCACCAAATCATCCTGAATCTGATCTTTTTATACACAATAT |
| | | ACGAAAAGCCAGCTTGAA |
| 19 | SmBiT | VTGYRLFEEIL |
| 20 | LgBiT | VFTLEDFVGDWEQTAAYNLDQVLEQGGVSSLLQNLAVSVTPIQRIVRSGENALKIDIH |
| | | VIIPYEGLSADQMAQIEEVFKVVYPVDDHHFKVILPYGTLVIDGVTPNMLNYFGRPYE |
| | | GIAVFDGKKITVTGTLWNGNKIIDERLITPDGSMLFRVTINS |
| 21 | Choloepus didactylus | MGRKRDAILEALENLTSDELKKFKLKLGVVPLREGFRNIPRGALGPLDAVDLTDKLVA |
| | ASC2 | FYREDYGAELTAAVLQDMGMQEEASQLQAVA |
| | (XM_037815384) | |
| 22 | Condylura cristata | MGKKRDAILETLENLTADELKKFKLKLGAAKLPEGFRVIPRGALGPLDVVDLTDKMVS |
| | ASC2 | FYREDFSAKLAADLLTDMGLQEEAARLQQLE |
| | (XM_004691672) | |
| 23 | Dasypus | MGKKRDAILEALENLTSDELKKFKLKLGVVPLREGFRNIPRGALGPLDPVDLTDKLVS |
| | novemcinctus ASC2 | FYREDYGAELAATVLLDMGMQEEASRLQAVA |
| | (XM_004479428) | |
| 24 | Galeopterus | MGGKRDAILEGLENLTADESKKFKLKLGAARLRQGFRNIPRGVLGPLDTVDLTDKLV |
| | variegatus ASC2 | AFYCEEYRAELTAVVLRDTGMQEEATRLQGAT |
| | (XM_008594345) | |
| 25 | Ictidomys | MGKKRDAILEALENLTTDELKKFKLKVGAVQLREGCRNIPRGALLPLDAVDLTDKLVA |
| | tridecemlineatus | FYREDYAAELTAAVLLDMGMQEEATRLKGVA |
| | ASC2 | |
| | (XM_005323886) | |
| 26 | Loxodonta Africana | MGKKRDAILEALESLTTDEFKKFKLKLGVVQLREGFRNIPRGALGPLDAVDL?KLVAF |
| | ASC2 | YREDYVAELTVAVLIDMGMQEEAVWLQGVA |
| | (XM_023558981) | |
| 27 | Marmot flaviventris | MGKKRDAILEALENLTTDELKKFKLKVGAVQLREGCRNIPRGALLPLDSVDLTDKLVA |
| | ASC2 | FYREDYAAELTAAVLLDMGMQEEATRLKGVA |
| | (XM_027948732) | |
| 28 | Marmota marmota | MGKKRDAILEALENLTTDELKKFKLKVGAVQLREGCRNIPRGALLPLDSVDLTDKLVA |
| | marmota ASC2 | FYREDYAAELTAAVLLDMGMQEEATRLKGVA |
| | (XM_015491354) | |
| 29 | Nannospalax galili | MANKRDAILEALENLTKEEFKKFKLKLGLVPLRQGFRRIPRGTLELVDAIDLTDRLVAF |
| | ASC2 | YCEDYGAELTATVLQDMGMQELAAQLRAVA |
| 30 | Notechis scutatus | MAESVRSTIGNVLEDLTEDELRKFKANLHDYQVKPSYNNIPRGRLQKADALDLSDLL |
| | ASC2 | VSFYNEDYAVEVAAAVLMASNCKPQAQRLLRAV |
| | (XM_008823790) | |
| 31 | Phascolarctos | MPGGRDLILTALDNLTEDEFKRFKSKLLTVPLRDGFNRIPRGILQNLDRLDLTDRIVSY |
| | cinereus ASC2 | YLEDYGTKLTVQVLRDIGMQQEAVRLQQAA |
| | (XM_021004576) | |
| 32 | Pseudonaja textilis | MAESVRSTIENVLEDLTEDELRKFKANLHDYQVKPGYNNIPRGQLQKADALDLSDLL |
| | ASC2 | VSFYNEDYAVEVAAAVLMASNCKPQAQRLLRAV |
| | (XM_026711966) | |
| 33 | Talpa occidentalis | MGKKRDAILEALENLTADELKKFKLKLGAANVREGYRTIPRGSLGILDAVDLTDKLVS |
| | ASC2 | FYREDYAAELTAGLLADMGLQEEAARLQGLA |
| | (XM_037507174) | |
| 34 | Tupaia chinensis | MGKKRDAILDALENLTTDELKKFKLKLGAARLPEGFKNIPRGALGQLDAVDLTDKLVS |
| | ASC2 | FYCEDYAAELTAAVLLDMGMREEAARLQGAA |
| | (XM_027771693) | |
| 35 | Urocitellus parryii | MGKKRDAILEALENLTTDELKKFKLKVGAVQLREGCRNIPRGALMPLDAVDLTDKLV |
| | ASC2 | VFYREDYAAELTAAVLLDMGMQEEATRLKGVA |
| | (XM_026400323) | |
| 36 | Vombatus ursinus | MPGGRDLILTALDNLTEDEFKRFKSKLLTVPLRDGFNRIPRGILHDLDRLDLTDRIISY |
| | ASC2 | YLEDYGTELTVQVLLDIGMREEAVRLQQAA |
| | (XM_027859094) | |

### Numbered Paragraphs

The following numbered paragraphs (paras) provide further statements of features and combinations of features which are contemplated in connection with the present invention:
1a. A polypeptide comprising or consisting of an amino acid sequence having at least 50% amino acid sequence identity to SEQ ID NO:6, wherein the amino acid sequence of the polypeptide is non-identical to the amino acid sequence of a naturally-occurring protein, and wherein the polypeptide inhibits interaction between ASC and an interaction partner for ASC.
2a. The polypeptide according to para 1a, wherein the polypeptide comprises one or more of: E or D at the position corresponding to position 10 of SEQ ID NO:6; R, K or H at the position corresponding position 37 of SEQ ID NO:6; C, R, K or H at the position corresponding position 61 of SEQ ID NO:6; and G at the position corresponding position 77 of SEQ ID NO:6.
3a. The polypeptide according to para 1a or para 2a, wherein the polypeptide comprises one or more of: E at the position corresponding to position 10 of SEQ ID NO:6; R at the position corresponding position 37 of SEQ ID NO:6; C at the position corresponding position 61 of SEQ ID NO:6; and G at the position corresponding position 77 of SEQ ID NO:6.
4a. The polypeptide according to any one of paras 1a to 3a, wherein the polypeptide comprises E or D at the position corresponding to position 10 of SEQ ID NO:6; R, K or H at the position corresponding position 37 of SEQ ID NO:6; C, R, K or H at the position corresponding position 61 of SEQ ID NO:6; and G at the position corresponding position 77 of SEQ ID NO:6.
5a. The polypeptide according to any one of paras 1a to 4a, wherein the polypeptide comprises E at the position corresponding to position 10 of SEQ ID NO:6; R at the position corresponding position 37 of SEQ ID NO: 6; C at the position corresponding position 61 of SEQ ID NO:6; and G at the position corresponding position 77 of SEQ ID NO:6.
6a. The polypeptide according to any one of paras 1a to 5a, wherein the polypeptide comprises or consists of the amino acid sequence of SEQ ID NO:6.
7a. The polypeptide according to any one of paras 1a to 6a, wherein the polypeptide further comprises a cell-penetrating peptide.
8a. A composition comprising a polypeptide according to any one of paras 1a to 7a, encapsulated in, or immobilised on, a nanoparticle, liposome, nanogel or nanolipogel.
9a. A nucleic acid encoding a polypeptide according to any one of paras 1a to 7a.
10a. An expression vector, comprising a nucleic acid according to para 9a.
11a. A cell comprising a polypeptide according to any one of paras 1a to 7a, a composition according to para 8a, a nucleic acid according to para 9a, or an expression vector according to para 10a.
12a. A method for producing a polypeptide according to any one of paras 1a to 7a, comprising culturing a cell comprising a nucleic acid according to para 9a, or an expression vector according to para 10a, under conditions suitable for expression of the polypeptide by the cell.
13a. A pharmaceutical composition comprising a polypeptide according to any one of paras 1a to 7a, a composition according to para 8a, a nucleic acid according to para 9a, an expression vector according to para 10a, or a cell according to para 11a, and a pharmaceutically acceptable carrier, diluent, excipient or adjuvant.
14a. A polypeptide according to any one of paras 1a to 7a, a composition according to para 8a, a nucleic acid according to para 9a, an expression vector according to para 10a, a cell according to para 11a, or a pharmaceutical composition according to para 13a, for use in a method of medical treatment or prophylaxis.
15a. A (n)
   (i) polypeptide comprising or consisting of an amino acid sequence having at least 50% amino acid sequence identity to SEQ ID NO:6, 7, 8, 9, 10, 11, 12, 13, 14, 15, 16 or 17 which inhibits interaction between ASC and an interaction partner for ASC;
   (ii) polypeptide according to (i), wherein the polypeptide further comprises a cell-penetrating peptide;
   (iii) composition comprising a polypeptide according to (i) or (ii), encapsulated in, or immobilised on, a nanoparticle, liposome, nanogel or nanolipogel;
   (iv) nucleic acid encoding a polypeptide according to (i) or (ii);
   (v) expression vector comprising a nucleic acid according to (iv);
   (vi) cell comprising: (a) a polypeptide according to (i) or (ii), (b) a composition according to (iii), (c) a nucleic acid according to (iv), or (d) an expression vector according to (v); or
   (vii) pharmaceutical composition comprising: (a) a polypeptide according to (i) or (ii), (b) a composition according to (iii), (c) a nucleic acid according to (iv), (d) an expression vector according to (v), or (e) a cell according to (vi), and a pharmaceutically acceptable carrier, diluent, excipient or adjuvant;
   for use in a method of medical treatment or prophylaxis.
16a. A polypeptide according to any one of paras 1a to 7a, a composition according to para 8a, a nucleic acid according to para 9a, an expression vector according to para 10a, a cell according to para 11a, or a pharmaceutical composition according to para 13a, for use in a method of treating or preventing a disease or condition in which inflammasome activity is pathologically-implicated.
17a. Use of a polypeptide according to any one of paras 1a to 7a, a composition according to para 8a, a nucleic acid according to para 9a, an expression vector according to para 10a, a cell according to para 11a, or a pharmaceutical composition according to para 13a, in the manufacture of a medicament for treating or preventing a disease or condition in which inflammasome activity is pathologically-implicated.
18a. A method of treating or preventing a disease or condition in which inflammasome activity is pathologically-implicated, comprising administering a therapeutically- or prophylactically-effective amount of a polypeptide according to any one of paras 1a to 7a, a composition according to para 8a, a nucleic acid according to para 9a, an expression vector according to para 10a, a cell according to para 11a, or a pharmaceutical composition according to para 13a, to a subject in need thereof.
19a. A (n)
   (i) polypeptide comprising or consisting of an amino acid sequence having at least 50% amino acid sequence identity to SEQ ID NO: 6, 7, 8, 9, 10, 11, 12, 13, 14, 15, 16 or 17 which inhibits interaction between ASC and an interaction partner for ASC;
   (ii) polypeptide according to (i), wherein the polypeptide further comprises a cell-penetrating peptide;
   (iii) composition comprising a polypeptide according to (i) or (ii), encapsulated in, or immobilised on, a nanoparticle, liposome, nanogel or nanolipogel;
   (iv) nucleic acid encoding a polypeptide according to (i) or (ii);
   (v) expression vector comprising a nucleic acid according to (iv);
   (vi) cell comprising: (a) a polypeptide according to (i) or (ii), (b) a composition according to (iii), (c) a nucleic acid according to (iv), or (d) an expression vector according to (v); or
   (vii) pharmaceutical composition comprising: (a) a polypeptide according to (i) or (ii), (b) a composition according to (iii), (c) a nucleic acid according to (iv), (d) an expression vector according to (v), or (e) a cell according to (vi), and a pharmaceutically acceptable carrier, diluent, excipient or adjuvant;
   for use in a method of treating or preventing a disease or condition in which inflammasome activity is pathologically-implicated.
20a. Use of:
   (i) a polypeptide comprising or consisting of an amino acid sequence having at least 50% amino acid sequence identity to SEQ ID NO:6, 7, 8, 9, 10, 11, 12, 13, 14, 15, 16 or 17 which inhibits interaction between ASC and an interaction partner for ASC;
   (ii) polypeptide according to (i), wherein the polypeptide further comprises a cell-penetrating peptide;
   (iii) a composition comprising a polypeptide according to (i) or (ii), encapsulated in, or immobilised on, a nanoparticle, liposome, nanogel or nanolipogel;
   (iv) a nucleic acid encoding a polypeptide according to (i) or (ii);
   (v) an expression vector comprising a nucleic acid according to (iv);
   (vi) a cell comprising: (a) a polypeptide according to (i) or (ii), (b) a composition according to (iii), (c) a nucleic acid according to (iv), or (d) an expression vector according to (v); or
   (vii) a pharmaceutical composition comprising: (a) a polypeptide according to (i) or (ii), (b) a composition according to (iii), (c) a nucleic acid according to (iv), (d) an expression vector according to (v), or (e) a cell according to (vi), and a pharmaceutically acceptable carrier, diluent, excipient or adjuvant;
   in the manufacture of a medicament for treating or preventing a disease or condition in which inflammasome activity is pathologically-implicated.
21a. A method of treating or preventing a disease or condition in which inflammasome activity is pathologically-implicated, comprising administering a therapeutically- or prophylactically-effective amount of:
   (i) a polypeptide comprising or consisting of an amino acid sequence having at least 50% amino acid sequence identity to SEQ ID NO:6, 7, 8, 9, 10, 11, 12, 13, 14, 15, 16 or 17 which inhibits interaction between ASC and an interaction partner for ASC;
   (ii) polypeptide according to (i), wherein the polypeptide further comprises a cell-penetrating peptide;
   (iii) a composition comprising a polypeptide according to (i) or (ii), encapsulated in, or immobilised on, a nanoparticle, liposome, nanogel or nanolipogel;
   (iv) a nucleic acid encoding a polypeptide according to (i) or (ii);
   (v) an expression vector comprising a nucleic acid according to (iv);
   (vi) a cell comprising: (a) a polypeptide according to (i) or (ii), (b) a composition according to (iii), (c) a nucleic acid according to (iv), or (d) an expression vector according to (v); or
   (vii) a pharmaceutical composition comprising: (a) a polypeptide according to (i) or (ii), (b) a composition according to (iii), (c) a nucleic acid according to (iv), (d) an expression vector according to (v), or (e) a cell according to (vi), and a pharmaceutically acceptable carrier, diluent, excipient or adjuvant;
   to a subject in need thereof.
22a. The polypeptide, composition, nucleic acid, expression vector, cell or pharmaceutical composition for use according to para 16a or para 19a, the use according to para 17a or para 20a, or the method according to para 18a or para 21a, wherein the disease or condition in which inflammasome activity is pathologically-implicated is selected from: an inflammatory disease, a chronic inflammatory disease, a neurodegenerative disease, Alzheimer's disease, Parkinson's disease, an autoimmune disease, multiple sclerosis, rheumatoid arthritis, systemic lupus erythematosus (SLE), an autoinflammatory disease, cryopyrin-associated periodic syndrome (CAPS), familial cold autoinflammatory syndrome (FCAS), Muckle-Wells syndrome (MWS), neonatal onset multisystemic inflammatory disease (NOMID), chronic infantile neurologic cutaneous articular (CINCA), a cancer, myelodysplastic syndrome, a cardiovascular disease, atherosclerosis, acute myocardial infarction, stroke, hypertension, an ocular disease, glaucoma, acute glaucoma, macular degeneration, age-related macular degeneration, a respiratory disease, asthma, severe steroid-resistant asthma, chronic obstructive pulmonary disease (COPD), respiratory disease cause by viral infection, gout, a metabolic disease, obesity-induced insulin resistance, diabetes, type 1 diabetes, type 2 diabetes, non-alcoholic fatty liver disease (NAFLD), silicosis, crystal nephropathy, a skin disease, acne, atopic dermatitis, psoriasis, vitiligo, contact hypersensitivity, inflammatory disease caused by viral infection, inflammatory bowel disease, graft-versus-host disease and traumatic brain injury.
23a. A method for inhibiting an inflammasome, comprising introducing into a cell, in vitro or in vivo:
   (i) a polypeptide comprising or consisting of an amino acid sequence having at least 50% amino acid sequence identity to SEQ ID NO: 6, 7, 8, 9, 10, 11, 12, 13, 14, 15, 16 or 17 which inhibits interaction between ASC and an interaction partner for ASC;
   (ii) polypeptide according to (i), wherein the polypeptide further comprises a cell-penetrating peptide;
   (iii) a composition comprising a polypeptide according to (i) or (ii), encapsulated in, or immobilised on, a nanoparticle, liposome, nanogel or nanolipogel;
   (iv) a nucleic acid encoding a polypeptide according to (i) or (ii); or
   (v) an expression vector comprising a nucleic acid according to (iv).
24a. Use of:
   (i) a polypeptide comprising or consisting of an amino acid sequence having at least 50% amino acid sequence identity to SEQ ID NO: 6, 7, 8, 9, 10, 11, 12, 13, 14, 15, 16 or 17 which inhibits interaction between ASC and an interaction partner for ASC;
   (ii) polypeptide according to (i), wherein the polypeptide further comprises a cell-penetrating peptide;
   (iii) a composition comprising a polypeptide according to (i) or (ii), encapsulated in, or immobilised on, a nanoparticle, liposome, nanogel or nanolipogel;
   (iv) a nucleic acid encoding a polypeptide according to (i) or (ii); or
   (v) an expression vector comprising a nucleic acid according to (iv);
   in an in vitro or in vivo method for inhibiting an inflammasome.
25a. The polypeptide, composition, nucleic acid, expression vector, cell or pharmaceutical composition for use, the use, or the method according to any one of paras 15a or 19a to 24a, wherein the polypeptide comprises one or more of: E or D at the position corresponding to position 10 of SEQ ID NO:6; R, K or H at the position corresponding position 37 of SEQ ID NO:6; C, R, K or H at the position corresponding position 61 of SEQ ID NO:6; and G at the position corresponding position 77 of SEQ ID NO:6.
26a. The polypeptide, composition, nucleic acid, expression vector, cell or pharmaceutical composition for use, the use, or the method according to any one of paras 15a or 19a to 25a, wherein the polypeptide comprises one or more of: E at the position corresponding to position 10 of SEQ ID NO:6; R at the position corresponding position 37 of SEQ ID NO:6; C at the position corresponding position 61 of SEQ ID NO: 6; and G at the position corresponding position 77 of SEQ ID NO:6.
27a. The polypeptide, composition, nucleic acid, expression vector, cell or pharmaceutical composition for use, the use, or the method according to any one of paras 15a or 19a to 26a, wherein the polypeptide comprises E or D at the position corresponding to position 10 of SEQ ID NO:6; R, K or H at the position corresponding position 37 of SEQ ID NO:6; C at the position corresponding position 61 of SEQ ID NO:6; and G at the position corresponding position 77 of SEQ ID NO:6.
28a. The polypeptide, composition, nucleic acid, expression vector, cell or pharmaceutical composition for use, the use, or the method according to any one of paras 15a or 19a to 27a, wherein the polypeptide comprises E at the position corresponding to position 10 of SEQ ID NO:6; R at the position corresponding position 37 of SEQ ID NO:6; C at the position corresponding position 61 of SEQ ID NO:6; and G at the position corresponding position 77 of SEQ ID NO:6.
29a. The polypeptide, composition, nucleic acid, expression vector, cell or pharmaceutical composition for use, the use, or the method according to any one of paras 15a or 19a to 28a, wherein the polypeptide comprises or consists of the amino acid sequence of SEQ ID NO:6.

The following further numbered paragraphs (paras) provide further statements of features and combinations of features which are contemplated in connection with the present invention:
1b. An ASC inhibitor for use in a method of treating or preventing a disease or condition in which inflammasome activity is pathologically-implicated.
2b. Use of an ASC inhibitor in the manufacture of a medicament for use in a method of treating or preventing a disease or condition in which inflammasome activity is pathologically-implicated.
3b. A method of treating or preventing a disease or condition in which inflammasome activity is pathologically-implicated, comprising administering a therapeutically- or prophylactically-effective amount of an ASC inhibitor to a subject.
4b. The ASC inhibitor for use according to para 1b, the use according to para 2b, or the method according to para 3b, wherein the ASC inhibitor is an ASC-binding molecule, an ASC target-binding molecule, or a molecule capable of reducing expression of ASC.
5b. The ASC inhibitor for use according to para 1b or para 4b, the use according to para 2b or para 4b, or the method according to para 3b or para 4b, wherein the ASC inhibitor is capable of preventing or reducing the binding of ASC to an interaction partner of ASC.
6b. The ASC inhibitor for use according to any one of paras 1b, 4b or 5b, the use according to any one of paras 2b, 4b or 5b, or the method according to any one of paras 3b to 5b, wherein the ASC inhibitor is selected from the group consisting of: an antibody or an antigen-binding fragment thereof, a polypeptide, a peptide, a nucleic acid, an oligonucleotide, an aptamer or a small molecule.
7b. The ASC inhibitor for use according to para 1b or para 4b, the use according to para 2b or para 4b, or the method according to para 3b or para 4b, wherein the ASC inhibitor is capable of preventing or reducing the expression of ASC.
8b. The ASC inhibitor for use, the use, or the method according to para 7b, wherein the ASC inhibitor is an oligonucleotide or a small molecule.
9b. A method of identifying an ASC inhibitor, comprising:
   contacting a putative ASC inhibitor with a reporter system, wherein the reporter system comprises: (i) an ASC polypeptide or a domain thereof linked to a first reporter component, and (ii) an interaction partner for the ASC polypeptide or a domain thereof of (i) linked to a second reporter component, wherein upon interaction between (i) and (ii) a reporter signal is produced; and
   wherein a putative ASC inhibitor that reduces the level of the reporter signal is identified as an ASC inhibitor.
10b. The method according to para 9b, wherein (i) and/or (ii) comprise:
   (a) a PYD domain, optionally wherein the PYD domain comprises an amino acid sequence having at least 50% amino acid sequence identity to the amino acid sequence of SEQ ID NO:4; and/or
   (b) a CARD domain, optionally wherein the CARD domain comprises an amino acid sequence having at least 50% amino acid sequence identity to the amino acid sequence of SEQ ID NO:5.
11b. The method of para 9b or 10b wherein the reporter components of (i) and (ii) are components of a protein-fragment complementation system or a resonance energy transfer system.
12b. The method of para 11b, wherein the protein-fragmentation complementation system is selected from a yeast-two hybrid system, split ubiquitin system, split luciferase system, biomolecular fluorescence complementation system, and bimolecular luciferase complementation system,
   optionally wherein the biomolecular fluorescence complementation system is a split Venus system,
   optionally wherein the biomolecular luciferase complementation system is a NanoLuc Binary Technology (NanoBiT) system.
13b. The method of para 11b, wherein the resonance energy transfer system is selected from a FRET system and a BRET system, optionally wherein the BRET system is a NanoBRET system.
14b. The method of any of paras 9b to 12b, wherein the reporter system comprises a NanoBIT system wherein (i) comprises a LgBiT moiety and (ii) comprises a SmBiT moiety or (i) comprises a SmBiT moiety and (ii) comprises a LgBiT moiety.
15b. The method of any of paras 9b to 12b, wherein the reporter system comprises a split Venus system wherein (i) comprises a VN moiety and (ii) comprises a VC moiety, or (i) comprises a VC moiety and (ii) comprises a VN moiety, optionally wherein the VN moiety is a VN173 moiety and/or the VC moiety is a VC155 moiety.
16b. The method according to any one of paras 9b to 15b, wherein the method comprises detecting the level of the reporter signal.
17b. The method according to any one of paras 9b to 16b, wherein the method comprises determining whether the putative ASC inhibitor reduces the level of the reporter signal.
18b. An ASC inhibitor identified by the method according to any one of paras 9b to 17b.
19b. The ASC inhibitor according to para 18b, for use in a method of treating or preventing a disease or condition in which inflammasome activity is pathologically-implicated.
20b. Use of an ASC inhibitor according to para 18b in the manufacture of a medicament for use in a method of treating or preventing a disease or condition in which inflammasome activity is pathologically-implicated.
21b. A method of treating or preventing a disease or condition in which inflammasome activity is pathologically-implicated, comprising administering a therapeutically- or prophylactically-effective amount of an ASC inhibitor according to para 18b to a subject.
22b. The ASC inhibitor for use, the use or the method according to any one of paras 1b to 8b or 19b to 21b, wherein the disease or condition in which inflammasome activity is pathologically-implicated is selected from: an inflammatory disease, a chronic inflammatory disease, a neurodegenerative disease, Alzheimer's disease, Parkinson's disease, an autoimmune disease, multiple sclerosis, rheumatoid arthritis, systemic lupus erythematosus (SLE), an autoinflammatory disease, cryopyrin-associated periodic syndrome (CAPS), familial cold autoinflammatory syndrome (FCAS), Muckle-Wells syndrome (MWS), neonatal onset multisystemic inflammatory disease (NOMID), chronic infantile neurologic cutaneous articular (CINCA), a cancer, myelodysplastic syndrome, a cardiovascular disease, atherosclerosis, acute myocardial infarction, stroke, hypertension, an ocular disease, glaucoma, acute glaucoma, macular degeneration, age-related macular degeneration, a respiratory disease, asthma, severe steroid-resistant asthma, chronic obstructive pulmonary disease (COPD), respiratory disease cause by viral infection, gout, a metabolic disease, obesity-induced insulin resistance, diabetes, type 1 diabetes, type 2 diabetes, non-alcoholic fatty liver disease (NAFLD), silicosis, crystal nephropathy, a skin disease, acne, atopic dermatitis, psoriasis, vitiligo, contact hypersensitivity, inflammatory disease caused by viral infection, inflammatory bowel disease, graft-versus-host disease and traumatic brain injury.

The following further numbered paragraphs (paras) provide further statements of features and combinations of features which are contemplated in connection with the present invention:
1c. A (n)
   (i) polypeptide comprising or consisting of an amino acid sequence having at least 50% amino acid sequence identity to SEQ ID NO:6, 7, 8, 9, 10, 11, 12, 13, 14, 15, 16, 17, 21, 22, 23, 24, 25, 26, 27, 28, 29, 30, 31, 32, 33, 34, 35 or 36 which inhibits interaction between ASC and an interaction partner for ASC;
   (ii) polypeptide according to (i), wherein the polypeptide further comprises a cell-penetrating peptide;
   (iii) composition comprising a polypeptide according to (i) or (ii), encapsulated in, or immobilised on, a nanoparticle, liposome, nanogel or nanolipogel;
   (iv) nucleic acid encoding a polypeptide according to (i) or (ii);
   (v) expression vector comprising a nucleic acid according to (iv);
   (vi) cell comprising: (a) a polypeptide according to (i) or (ii), (b) a composition according to (iii), (c) a nucleic acid according to (iv), or (d) an expression vector according to (v); or
   (vii) pharmaceutical composition comprising: (a) a polypeptide according to (i) or (ii), (b) a composition according to (iii), (c) a nucleic acid according to (iv), (d) an expression vector according to (v), or (e) a cell according to (vi), and a pharmaceutically acceptable carrier, diluent, excipient or adjuvant;
   for use in a method of medical treatment or prophylaxis.
2c. A (n)
   (i) polypeptide comprising or consisting of an amino acid sequence having at least 50% amino acid sequence identity to SEQ ID NO: 6, 7, 8, 9, 10, 11, 12, 13, 14, 15, 16, 17, 21, 22, 23, 24, 25, 26, 27, 28, 29, 30, 31, 32, 33, 34, 35 or 36 which inhibits interaction between ASC and an interaction partner for ASC;
   (ii) polypeptide according to (i), wherein the polypeptide further comprises a cell-penetrating peptide;
   (iii) composition comprising a polypeptide according to (i) or (ii), encapsulated in, or immobilised on, a nanoparticle, liposome, nanogel or nanolipogel;
   (iv) nucleic acid encoding a polypeptide according to (i) or (ii);
   (v) expression vector comprising a nucleic acid according to (iv);
   (vi) cell comprising: (a) a polypeptide according to (i) or (ii), (b) a composition according to (iii), (c) a nucleic acid according to (iv), or (d) an expression vector according to (v); or
   (vii) pharmaceutical composition comprising: (a) a polypeptide according to (i) or (ii), (b) a composition according to (iii), (c) a nucleic acid according to (iv), (d) an expression vector according to (v), or (e) a cell according to (vi), and a pharmaceutically acceptable carrier, diluent, excipient or adjuvant;
   for use in a method of treating or preventing a disease or condition in which inflammasome activity is pathologically-implicated.
3c. Use of:
   (i) a polypeptide comprising or consisting of an amino acid sequence having at least 50% amino acid sequence identity to SEQ ID NO:6, 7, 8, 9, 10, 11, 12, 13, 14, 15, 16, 17, 21, 22, 23, 24, 25, 26, 27, 28, 29, 30, 31, 32, 33, 34, 35 or 36 which inhibits interaction between ASC and an interaction partner for ASC;
   (ii) polypeptide according to (i), wherein the polypeptide further comprises a cell-penetrating peptide;
   (iii) a composition comprising a polypeptide according to (i) or (ii), encapsulated in, or immobilised on, a nanoparticle, liposome, nanogel or nanolipogel;
   (iv) a nucleic acid encoding a polypeptide according to (i) or (ii);
   (v) an expression vector comprising a nucleic acid according to (iv);
   (vi) a cell comprising: (a) a polypeptide according to (i) or (ii), (b) a composition according to (iii), (c) a nucleic acid according to (iv), or (d) an expression vector according to (v); or
   (vii) a pharmaceutical composition comprising: (a) a polypeptide according to (i) or (ii), (b) a composition according to (iii), (c) a nucleic acid according to (iv), (d) an expression vector according to (v), or (e) a cell according to (vi), and a pharmaceutically acceptable carrier, diluent, excipient or adjuvant;
   in the manufacture of a medicament for treating or preventing a disease or condition in which inflammasome activity is pathologically-implicated.
4c. A method of treating or preventing a disease or condition in which inflammasome activity is pathologically-implicated, comprising administering a therapeutically- or prophylactically-effective amount of:
   (i) a polypeptide comprising or consisting of an amino acid sequence having at least 50% amino acid sequence identity to SEQ ID NO:6, 7, 8, 9, 10, 11, 12, 13, 14, 15, 16, 17, 21, 22, 23, 24, 25, 26, 27, 28, 29, 30, 31, 32, 33, 34, 35 or 36 which inhibits interaction between ASC and an interaction partner for ASC;
   (ii) polypeptide according to (i), wherein the polypeptide further comprises a cell-penetrating peptide;
   (iii) a composition comprising a polypeptide according to (i) or (ii), encapsulated in, or immobilised on, a nanoparticle, liposome, nanogel or nanolipogel;
   (iv) a nucleic acid encoding a polypeptide according to (i) or (ii);
   (v) an expression vector comprising a nucleic acid according to (iv);
   (vi) a cell comprising: (a) a polypeptide according to (i) or (ii), (b) a composition according to (iii), (c) a nucleic acid according to (iv), or (d) an expression vector according to (v); or
   (vii) a pharmaceutical composition comprising: (a) a polypeptide according to (i) or (ii), (b) a composition according to (iii), (c) a nucleic acid according to (iv), (d) an expression vector according to (v), or (e) a cell according to (vi), and a pharmaceutically acceptable carrier, diluent, excipient or adjuvant;
   to a subject in need thereof.
5c. The polypeptide, composition, nucleic acid, expression vector, cell or pharmaceutical composition for use according to para 2c, the use according to para 3c, or the method according to para 4c, wherein the disease or condition in which inflammasome activity is pathologically-implicated is selected from: an inflammatory disease, a chronic inflammatory disease, a neurodegenerative disease, Alzheimer's disease, Parkinson's disease, an autoimmune disease, multiple sclerosis, rheumatoid arthritis, systemic lupus erythematosus (SLE), an autoinflammatory disease, cryopyrin-associated periodic syndrome (CAPS), familial cold autoinflammatory syndrome (FCAS), Muckle-Wells syndrome (MWS), neonatal onset multisystemic inflammatory disease (NOMID), chronic infantile neurologic cutaneous articular (CINCA), a cancer, myelodysplastic syndrome, a cardiovascular disease, atherosclerosis, acute myocardial infarction, stroke, hypertension, an ocular disease, glaucoma, acute glaucoma, macular degeneration, age-related macular degeneration, a respiratory disease, asthma, severe steroid-resistant asthma, chronic obstructive pulmonary disease (COPD), respiratory disease cause by viral infection, gout, a metabolic disease, obesity-induced insulin resistance, diabetes, type 1 diabetes, type 2 diabetes, non-alcoholic fatty liver disease (NAFLD), silicosis, crystal nephropathy, a skin disease, acne, atopic dermatitis, psoriasis, vitiligo, contact hypersensitivity, inflammatory disease caused by viral infection, inflammatory bowel disease, graft-versus-host disease and traumatic brain injury.
6c. A method for inhibiting an inflammasome, comprising introducing into a cell, in vitro or in vivo:
   (i) a polypeptide comprising or consisting of an amino acid sequence having at least 50% amino acid sequence identity to SEQ ID NO: 6, 7, 8, 9, 10, 11, 12, 13, 14, 15, 16, 17, 21, 22, 23, 24, 25, 26, 27, 28, 29, 30, 31, 32, 33, 34, 35 or 36 which inhibits interaction between ASC and an interaction partner for ASC;
   (ii) polypeptide according to (i), wherein the polypeptide further comprises a cell-penetrating peptide;
   (iii) a composition comprising a polypeptide according to (i) or (ii), encapsulated in, or immobilised on, a nanoparticle, liposome, nanogel or nanolipogel;
   (iv) a nucleic acid encoding a polypeptide according to (i) or (ii); or
   (v) an expression vector comprising a nucleic acid according to (iv).
7c. Use of:
   (i) a polypeptide comprising or consisting of an amino acid sequence having at least 50% amino acid sequence identity to SEQ ID NO: 6, 7, 8, 9, 10, 11, 12, 13, 14, 15, 16, 17, 21, 22, 23, 24, 25, 26, 27, 28, 29, 30, 31, 32, 33, 34, 35 or 36 which inhibits interaction between ASC and an interaction partner for ASC;
   (ii) polypeptide according to (i), wherein the polypeptide further comprises a cell-penetrating peptide;
   (iii) a composition comprising a polypeptide according to (i) or (ii), encapsulated in, or immobilised on, a nanoparticle, liposome, nanogel or nanolipogel;
   (iv) a nucleic acid encoding a polypeptide according to (i) or (ii); or
   (v) an expression vector comprising a nucleic acid according to (iv);
   in an in vitro or in vivo method for inhibiting an inflammasome.
8c. The polypeptide, composition, nucleic acid, expression vector, cell or pharmaceutical composition for use, the use, or the method according to any one of paras 1c to 7c, wherein the polypeptide comprises one or more of: E or D at the position corresponding to position 10 of SEQ ID NO:6; R, K or H at the position corresponding position 37 of SEQ ID NO:6; C, R, K or H at the position corresponding position 61 of SEQ ID NO:6; and G at the position corresponding position 77 of SEQ ID NO:6.
9c. The polypeptide, composition, nucleic acid, expression vector, cell or pharmaceutical composition for use, the use, or the method according to any one of paras 1c to 8c, wherein the polypeptide comprises one or more of: E at the position corresponding to position 10 of SEQ ID NO:6; R at the position corresponding position 37 of SEQ ID NO:6; C at the position corresponding position 61 of SEQ ID NO:6; and G at the position corresponding position 77 of SEQ ID NO:6.
10c. The polypeptide, composition, nucleic acid, expression vector, cell or pharmaceutical composition for use, the use, or the method according to any one of paras 1c to 9c, wherein the polypeptide comprises E or D at the position corresponding to position 10 of SEQ ID NO:6; R, K or H at the position corresponding position 37 of SEQ ID NO:6; C at the position corresponding position 61 of SEQ ID NO:6; and G at the position corresponding position 77 of SEQ ID NO:6.
11c. The polypeptide, composition, nucleic acid, expression vector, cell or pharmaceutical composition for use, the use, or the method according to any one of paras 1c to 10c, wherein the polypeptide comprises E at the position corresponding to position 10 of SEQ ID NO:6; R at the position corresponding position 37 of SEQ ID NO:6; C at the position corresponding position 61 of SEQ ID NO:6; and G at the position corresponding position 77 of SEQ ID NO:6.
12c. The polypeptide, composition, nucleic acid, expression vector, cell or pharmaceutical composition for use, the use, or the method according to any one of paras 1c to 11c, wherein the polypeptide comprises or consists of the amino acid sequence of SEQ ID NO:6.

The present disclosure includes the combination of the aspects and preferred features described except where such a combination is clearly impermissible or expressly avoided.

The section headings used herein are for organisational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise,’ and variations such as ‘comprises’ and ‘comprising,’ will be understood to imply the inclusion of a stated integer or step or group of integers or steps but not the exclusion of any other integer or step or group of integers or steps.

It must be noted that, as used in the specification and the appended claims, the singular forms ‘a,’ ‘an,’ and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about,’ it will be understood that the particular value forms another embodiment.

Where a nucleic acid sequence is disclosed herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

### BRIEF DESCRIPTION OF THE FIGURES

Embodiments and experiments illustrating the principles of the present disclosure will now be discussed with reference to the accompanying figures.

FIGS. 1A-1H. Bat ASC2 is highly expressed at the mRNA level endogenously and at the protein level when overexpressed. (1A) Schematic showing maximum likelihood phylogenetic tree of ASC and ASC2 orthologs from four primates (H. sapiens, P. troglodytes, P. coquereli and S. boliviensis), six bats including four from Yinpterochiroptera and two from Yangochiroptera (P. alecto, R. ferrumequinum, R. sinicus, H. armiger, E. fuscus, P. parnellii) and a marsupial (P. cinereus) as an outgroup. Scale bar represents 0.2 substitutions per site. (1B) Schematic showing the amino acid sequence alignment of ASC and ASC2 of bat (P. alecto) and human with identical residues highlighted in dark grey and mismatches in light grey. Arrowed lines, the boundaries for PYRIN or CARD domain. (1C and 1D) Graphs showing read-mapping analysis of bat ASC and ASC2 by RNA-seq read counts of the putative monocytes/macrophages (CD11b⁺MHC-II⁻) (1C) and dendritic cells (CD11b⁺MHC-II⁺) (1D) of P. alecto splenocytes. (1E and 1F) Bar charts showing fold changes measured by qPCR of empty vector (His alone), bat ASC2 and human ASC2 tagged with His, overexpressed in HEK293T cells (1E) and bat kidney cells (1F). Primers used recognize the common 3′ region encoding the C-terminal tag. (1G and 1H) Immunoblots and bar charts showing analysis of cell lysates of HEK293T cells (1G) and bat kidney cells (1H) transfected with empty vector (His alone), bat ASC2 and human ASC2 tagged with His. ASC2 protein expression is quantified with anti-His signal and normalized to β-actin. Data are presented as representative of five biological replicates (1C and 1D), mean±s.d. of three independent experiments (1E-1H), or representative of three independent experiments (1G and 1H).****P<0.0001, NS, not significant, by two-tailed unpaired t-test.

FIGS. 2A-2E. Analysis of ASC and ASC2 expression in P. alecto cells. (2A) Immunoblot analysis showing cell lysates of bat kidney cells (PaKi) overexpressing empty vector (His alone), bat ASC or bat ASC2 tagged with His. (2B) Histogram showing bat ASC and bat ASC2 staining on PaKi stably expressing empty vector (EV) (mPlum alone) (grey), bat ASC-mPlum (red) or bat ASC2-mPlum (blue) by flow cytometry. (2C) Graphs showing analysis of ASC and ASC2 expression by flow cytometry in CD11b⁺ and CD11b⁻ leukocytes of P. alecto CD44+ splenocytes, using monoclonal antibody specific to bat ASC and bat ASC2, respectively. CD11b^{pos}, red; CD11b^{neg}, blue; isotype (CD11b^{pos}), grey; isotype (CD11b^{neg}), turquoise. (2D and 2E) Representative cell images of P. alecto PBMCs acquired using confocal microscopy. CD11b, purple; bat ASC, red; bat ASC2, green; propidium iodide (PI), blue. Arrowheads, ASC specks. Scale bars, 10 μm (2D) and 2 μM (2E). Data are presented as representative of three independent experiments (2A-2E).

FIGS. 3A-3H. Bat ASC2 inhibits the human NLRP3 and AIM2 inflammasome with a higher potency than human ASC2. (3A) Images showing representative single-cell HEK293T cells transfected with human NLRP3-mCitrine, human ASC-mPlum and LSSmOrange (control), bat or human ASC2-LSSmOrange. Cells were acquired using ImageStream (top) or confocal microscopy (bottom). NLRP3-mCitrine, green; ASC2-LSSmOrange, yellow; ASC-mPlum, red; DAPI, blue. Arrowheads, ASC specks. Scale bars, 10 μm (top) and 20 μm (bottom). (3B) Bar chart showing quantification of ASC specks by ImageStream in HEK293T cells transfected without (control) or with bat NLRP3-mCitrine, bat ASC-mPlum and LSSmOrange (control), bat or human ASC2-LSSmOrange. (3C and 3D) Bar charts showing quantification of ASC specks by ImageStream in HEK293T cells transfected without (control) or with human NLRP3-mCitrine (3C) or human AIM2-mCitrine (3D), human ASC-mPlum and LSSmOrange (control), bat or human ASC2-LSSmOrange. (3E and 3F) Bar charts showing LDH release (3E) and secretion of IL-1β by ELISA (3F) in cell-free supernatants of THP-1 macrophages stably expressing LSSmOrange (control), bat or human ASC2-LSSmOrange, primed with LPS for 3 h, with or without stimulation by nigericin for 2 h. (3G and 3H) Bar charts showing LDH release (3G) and secretion of IL-1β by ELISA (3H) in cell-free supernatants of THP-1 macrophages as in 3E and 3F, primed with LPS for 3 h, with or without transfection of poly(dA:dT) for 4 h. Data are presented as representative of three independent experiments (3A), or mean±s.d. of three independent experiments (3B-3H). *P<0.05, **P<0.01, ***P<0.001, ****P<0.0001, NS, not significant, by two-tailed unpaired t-test (3B), one-way ANOVA (3C and 3D), or two-way ANOVA (3E to 3H) with Bonferroni's multiple comparisons test.

FIGS. 4A-4J. Bat ASC2 Inhibits human inflammasomes in THP-1 macrophage with a higher potency than human ASC2. (4A) Immunoblot showing analysis of cell lysates of THP-1 macrophages stably expressing LSSmOrange (control), bat or human ASC2-LSSmOrange, unprimed (−) or primed (+) with LPS for 3 h. (4B) Images showing representative single-cell THP-1 macrophages stably expressing LSSmOrange (control), bat or human ASC2-LSSmOrange. Cells were acquired using ImageStream. DAPI, blue; ASC2-LSSmOrange, yellow; ASC, red. Arrowheads, ASC specks. Scale bars, 10 μm. (4C) Bar chart showing quantification of ASC specks by ImageStream in THP-1 macrophages stably expressing LSSmOrange (control), bat or human ASC2-LSSmOrange, unprimed (Un.) or primed with LPS for 3 h, with or without stimulation by nigericin for 1 h. (4D and 4E) Bar charts showing LDH release (4D) and secretion of IL-1β by ELISA (4E) in cell-free supernatants of THP-1 macrophages stably expressing LSSmOrange (control), bat or human ASC2-LSSmOrange, primed with LPS for 3 h, with or without stimulation by MSU crystals for 4 h. (4F) Bar chart showing fold changes measured by qPCR of human ASC2 in wild-type THP-1 macrophages and THP-1 stably expressing human ASC2-LSSmOrange, with (+) or without (−) PMA differentiation for 16 h and LPS priming for 3 h. (4G) Immunoblot showing analysis of cell lysates of HEK293T overexpressing Hupa4 ASC2 as positive control, wild-type THP-1 macrophages and THP-1 stably expressing human ASC2-LSSmOrange, with (+) or without (−) PMA differentiation for 16 h and LPS priming for 3 h. (4H) Immunoblot showing analysis of cell lysates of THP-1 macrophages stably expressing LSSmOrange (control), bat or human ASC2-LSSmOrange, primed with LPS for 3 h. (4I and 4J) Bar charts showing LDH release (4I) and secretion of IL-1β by ELISA (4J) in cell-free supernatants of wild-type THP-1 macrophages, primed with LPS for 3 h, with or without stimulation by nigericin for 2 h. Data are presented as representative of three independent experiments (4A, 4B, 4F and 4G), or mean±s.d. of three independent experiments (4C-4E, 4I and 4J). *P<0.05, **P<0.01, ***P<0.001, ****P<0.0001, NS, not significant, by one-way ANOVA (4I and 4J) and two-way ANOVA with Bonferroni's multiple comparisons test (4C-4E).

FIGS. 5A-5G. Bat ASC2 significantly reduces cell death and IL-16 secretion in mouse macrophages. (5A) Immunoblot showing analysis of bat ASC2 in cell lysates of BMDMs from wild-type (WT) mice and bat ASC2/rtTA mice, unprimed (−) or primed (+) with LPS for 3 h, in the absence (−) or presence (+) of doxycycline (Dox). ASC2, bat ASC2 stained with monoclonal antibody specific to bat ASC2. Grey box, highlighted conditions of WT or bat ASC2/rtTA BMDMs with Dox induction. (5B and 5C) Bar charts showing LDH release (5B) and secretion of IL-1β by ELISA (5C) in cell-free supernatants of WT and bat ASC2/rtTA BMDMs, primed with LPS for 3 h, with or without stimulation by nigericin for 1 h, in the absence (−) or presence (+) of Dox. (5D and 5E) Bar charts showing LDH release (5D) and secretion of IL-1β by ELISA (5E) in cell-free supernatants of WT and bat ASC2/rtTA BMDMs, primed with LPS for 3 h, with or without stimulation by transfection of poly(dA:dT) for 4 h, in the absence (−) or presence (+) of Dox. (5F and 5G) Bar charts showing LDH release (5F) and secretion of IL-1β by ELISA (5G) in cell-free supernatants of WT and bat ASC2/rtTA BMDMs, primed with LPS for 3 h, with or without stimulation by transfection of flagellin for 4 h, in the absence (−) or presence (+) of Dox. Grey box, highlighted conditions of WT or bat ASC2/rtTA BMDMs with stimulation and Dox induction (5B-5G). Data are presented as representative of three biological replicates (n=3) in 5A, or mean±s.d. of three biological replicates (n=3) in 5B-5G. *P<0.05, ***P<0.001, ****P<0.0001, NS=non-significant, by two-way ANOVA with Bonferroni's multiple comparisons test (5B-5G).

FIGS. 6A-6E. Bat ASC2 inhibits mouse inflammasome in BMDMs. (6A) Schematic representation of the generation of bat ASC2/rtTA transgenic mice. Transgenic mice with TetO-batASC2-IRES-eGFP was generated first and subsequently crossed with CAG-rtTA3 transgenic mice to generate batASC2/rtTA double transgenic mice. Expression of bat ASC2 with IRES-linked GFP is induced by doxycycline (Dox) in bat ASC2/rtTA mice. (6B) Images showing representative single-cell BMDMs from wild-type (WT) mice and bat ASC2/rtTA mice, primed with LPS for 3 h, with stimulation by nigericin for 30 min, in the presence of Dox. Cells were acquired using ImageStream. DAPI, blue; GFP, green; ASC, red. Arrowheads, ASC specks. Scale bars, 10 μm. (6C) Bar chart showing quantification of ASC specks by ImageStream in WT and bat ASC2/rtTA BMDMs, primed with LPS for 3 h, with or without stimulation by nigericin for 30 min, in the absence (−) or presence (+) of Dox. Grey box, highlighted conditions of WT or bat ASC2/rtTA BMDMs with stimulation and Dox induction. (6D and 6E) Bar charts showing LDH release (6D) and secretion of IL-1β by ELISA (6E) in cell-free supernatants of WT and bat ASC2/rtTA BMDMs, primed with LPS for 3 h, with or without stimulation by MSU crystals for 16 h, in the absence (−) or presence (+) of Dox. Grey box, highlighted conditions of WT or bat ASC2/rtTA BMDMs with stimulation and Dox induction (6C-6E). Data are presented as representative of three biological replicates (n=3) in 6B, or mean±s.d. of three biological replicates (n=3) in 6C-6E. *P<0.05, ****P<0.0001, NS=non-significant, by two-way ANOVA with Bonferroni's multiple comparisons test (6C-6E).

FIGS. 7A-7E. Delivery of TAT-ASC2 suppresses inflammasome activation in THP-1 macrophages. (7A and 7B) Images showing coomassie staining (7A) and immunoblot analysis (7B) of the recombinant TAT-batASC2-His. Negative, empty lane. (7C) Immunoblot showing analysis of cell lysates of THP-1 macrophages, primed with LPS for 3 h, treated with different concentrations (1 μg, 5 μg and 10 μg) of TAT-His or TAT-bat ASC-His, followed by stimulation by nigericin for 2 h. (7D and 7E) Bar charts showing LDH release (7D) and secretion of IL-1β by ELISA (7E) in cell-free supernatants of THP-1 macrophages as treated in 7C. Data are presented as representative of three independent experiments (7A to 7C), or mean±s.d. of three independent experiments (7D to 7E). *P<0.05, ***P<0.001, **** p<0.0001, NS=non-significant, by one-way ANOVA with Bonferroni's multiple comparisons test (7D and 7E).

FIGS. 8A-8F. Expression of bat ASC2 attenuates peritonitis induced by MSU crystals and ASC specks. (8A) Graphs showing analysis of GFP expression by flow cytometry in peripheral blood cells from wild-type (WT) and bat ASC2/rtTA mice upon oral administration of Dox for 7 days. (8B and 8C) Graphs showing gating strategy (8B) and bar chart showing quantification by flow cytometry (8C) of inflammatory monocytes and neutrophils obtained by peritoneal lavages of WT and bat ASC2/CAG mice 4 h after intraperitoneal injection of PBS or MSU crystals (200 μg), upon oral administration of Dox for 7 days. (8D) Bar chart showing IL-1β secretion by ELISA in the cell-free peritoneal lavages of mice as treated in 8B and 8C. (8E) Bar chart showing quantification by flow cytometry of inflammatory monocytes and neutrophils obtained by peritoneal lavages of WT and bat ASC2/CAG mice 4 h after intraperitoneal injection of ASC specks (10⁵ particles), upon oral administration of Dox for 7 days. (8F) Bar chart showing IL-1β secretion by ELISA in the cell-free peritoneal lavages of mice as treated in 8E. Data are presented as representative of three biological replicates (n=3) in 8A and 8B, or mean±s.d. of three biological replicates (n=3) in 8C-8F. *P<0.05, **P<0.01, NS, not significant, by one-way ANOVA (8C and 8D) and two-tailed unpaired t-test (8E and 8F).

FIGS. 9A-9D. Analysis of GFP expression in immune cell populations of bat ASC2/rtTA mice. (9A) Graphs showing analysis of GFP expression by flow cytometry in peritoneal lavages cells from WT and bat ASC2/rtTA mice. (9B) Graphs showing gating strategy of immune cell populations of peritoneal lavage cells or peripheral blood cells from WT or bat ASC2/rtTA mice. (9C) Graphs showing analysis of GFP expression by flow cytometry in bone marrow (left) and spleen (right) cells from WT and bat ASC2/rtTA mice. (9D) Graphs showing gating strategy of immune cell populations of bone marrow or spleen cells WT or bat ASC2/rtTA mice. Data are presented as representative of at least three biological replicates (9A-9D).

FIGS. 10A-10N. Expression of bat ASC2 reduces virus-induced inflammation and mortality. (10A) Graph showing survival of WT (n=6) and bat ASC2/rtTA (n=6) mice infected intranasally with IAV (0.5×10⁵ pfu), one day after oral Dox administration. (10B and 10C) Bar charts showing IL-1β levels by ELISA (B) and viral titres (C) in the lung homogenates from the infected mice at 3 days post-infection with IAV. (10D) Bar chart showing secretion of IL-1β by ELISA in cell-free supernatants of WT and bat ASC2/rtTA BMDMs, primed with LPS for 3 h, followed by infection with IAV (MOI=1) for 24 h, in the presence of Dox. (10E) Bar charts showing viral titres in cell-free supernatants of WT and bat ASC2/rtTA BMDMs primed with LPS for 3 h, followed by infection with IAV (MOI=1) (left) or PRV2M (MOI=5) (right) for 24 h, in the presence of Dox, expressed as p.f.u./ml (left) or TCID50/ml (right). (10F) Bar chart showing secretion of IL-1β by ELISA in cell-free supernatants of WT and bat ASC2/rtTA BMDMs, infected with ZIKV (MOI=1) for 24 h, in the presence of Dox. (10G) Bar chart showing secretion of IL-1β by ELISA in cell-free supernatants of WT and bat ASC2/rtTA BMDMs, primed with LPS for 3 h, followed by infection with PRV3M (MOI=5) for 24 h, in the presence of Dox. (10H) Schematic diagram showing monocytes treated by SARS-COV-2 immune complexes formed by coated RBD of SARS-COV-2 spike protein and anti-RBD monoclonal antibody. (10I) Graph showing secretion of IL-1β by ELISA in cell-free supernatants of human monocytes, primed with different concentrations of Pam3CSK4 (0, 1, and 100 ng/ml), activated by afucosylated or fucosylated SARS-COV-2 immune complexes, with or without 50 μM MCC950 for 18 h. (10J) Bar chart showing secretion of IL-1β by ELISA in cell-free supernatants of human monocytes, treated with 50 μM MCC950, 8 μM TAT-His (TAT-His) or TAT-batASC2-His (TAT-bat ASC2) for 1 h followed by priming with 100 ng/ml Pam3CSK4 and stimulation by SARS-COV-2 RBD only (RBD only), monoclonal antibody only (mAb only), or afucosylated SARS-COV-2 immune complexes (IC) for 2 h. (10K and 10L) Graph and bar chart showing representative mass spectra of glycopeptides encompassing the Fc region of SARS-COV-2 RBD monoclonal antibody CR3022 with or without α1-2,4,6 fucosidase O treatment. (10M) Bar chart showing LDH release in cell-free supernatants of human monocytes, primed with 100 ng/ml Pam3CSK4, activated by afucosylated or fucosylated SARS-COV-2 immune complexes, with or without 50 μM MCC950 for 18 h. (10N) Bar chart showing LDH release in cell-free supernatants of human monocytes, treated with 50 μM MCC950, 8 μM TAT-His (TAT) or TAT-batASC2-His (TAT-bat ASC2) for 1 h followed by priming with 100 ng/ml Pam3CSK4 and stimulation by SARS-COV-2 RBD only (RBD only), CR3022 monoclonal antibody only (mAb only), or afucosylated SARS-COV-2 immune complexes (IC) for 2 h. Data are presented as mean±s.d. of three biological replicates (n=3) in 10B-10G, 10J. Data are representative of three biological replicates (n=3) in 10I. Data are presented as mean±s.d. of three biological replicates (n=3) in 10E, 10M, 10N. *P<0.05, **P<0.01, ***P<0.001, ****P<0.0001, NS, not significant, by log-rank (Mantel-Cox) test (10A), two-tailed unpaired t-test (10B, 10C and 10E), one-way ANOVA (10J, 10M and 10N) or two-way ANOVA with Bonferroni's multiple comparisons test (10D, 10F and 10G).

FIGS. 11A-11M. Mutant human ASC2 (hupa4 ASC2) with changes of the 4 key residues in bat ASC2 exhibits increased expression and gain-of-function. (11A) Schematic representation showing ten chimaeras between bat (grey) and human (white) ASC2. ASC2 was divided into five regions based on its α-helical structures. Grey box, highlighted chimera 2. (11B and 11C) Bar charts showing quantification of ASC speck (11B) and filament formation (11C) by ImageStream in HEK293T cells transfected with human NLRP3-mCitrine, human ASC-mPlum and LSSmOrange (control), bat or human ASC2 or chimera-LSSmOrange. C1-C10, chimera 1 to chimera 10 as shown in 11A. (11D and 11E) Bar charts showing quantification of ASC specks (11D) and filament formation (11E) by ImageStream in HEK293T cell transfected with human NLRP3-mCitrine, human ASC-mPlum and LSSmOrange (control), bat or human ASC2 or mutant ASC2-LSSmOrange. Grey box, highlighted the mutants for the four key residues. (11F) Bar charts showing quantification of ASC specks by ImageStream in HEK293T cell transfected with human NLRP3-mCitrine, human ASC-mPlum and LSSmOrange (control), bat or primate ASC2-LSSmOrange. (11G) Immunoblot and bar chart showing analysis of cell lysates of HEK293T cells transfected with empty vector (His alone), bat or human ASC2 and hupa4 ASC2 tagged with His. (11H and 11I) Bar charts showing quantification of ASC speck (11H) and filament formation (11I) by ImageStream in HEK293T cells transfected with human NLRP3-mCitrine, human ASC-mPlum and LSSmOrange (control), bat or human ASC2 or hupa4 ASC2-LSSmOrange. (11J and 11K) Bar charts showing LDH release (11J) and secretion of IL-1β by ELISA (11K) in cell-free supernatants of mouse immortalized macrophages (iMACs) stably expressing LSSmOrange (control), bat or human ASC2 or hupa4 ASC2-LSSmOrange, primed with LPS for 3 h, with or without stimulation by nigericin for 1 h. (11L and 11M) Bar charts showing LDH release (11L) and secretion of IL-1β by ELISA (11M) in cell-free supernatants of THP-1 macrophages stably expressing mCitrine (control), bat or human ASC2 or hupa4 ASC2-mCitrine, primed with LPS for 3 h, with or without stimulation by nigericin for 2 h. Data are presented as mean±s.d. of three independent experiments (11B-11M), or as representative of three independent experiments (11G). *P<0.05, **P<0.01, ***P<0.001, ****P<0.0001, NS, not significant; one-way ANOVA (11B-11F and 11G-11I), or two-way ANOVA (11J-11M) with Bonferroni's multiple comparisons test.

FIGS. 12A-12M. The four key residues are critical in the enhanced expression and function of bat ASC2. (12A) Schematic representation showing twenty chimaeras (11-30) between bat (grey) and human (white) ASC2. ASC2 was divided into five regions based on its α-helical structures. (12B and 12C) Bar charts showing quantification of ASC-speck (12B) and filament formation (12C) by ImageStream in HEK293T cells transfected with human NLRP3-mCitrine, human ASC-mPlum and LSSmOrange (control), bat or human ASC2 or chimeras-LSSmOrange. C11-C30, chimera 11 to chimera 30 as shown in 12A. (12D) Bar charts showing quantification of filament formation by ImageStream in HEK293T cell transfected with human NLRP3-mCitrine, human ASC-mPlum and LSSmOrange (control), bat or primate ASC2-LSSmOrange. (12E and 12F) Bar charts showing fold changes measured by qPCR of empty vector (His alone), bat or human ASC2 or hupa4 ASC2 tagged with His, overexpressed in HEK293T cells (12E) and bat kidney cells (12F). (12G) Immunoblot and bar chart showing analysis of cell lysates of bat kidney cells transfected with empty vector (His alone), bat or human ASC2 or hupa4 ASC2 tagged with His. (12H and 12I) Images showing representative single-cell HEK293T cells transfected with human NLRP3-mCitrine, human ASC-mPlum and LSSmOrange (control), bat or human ASC2 or hupa4 ASC2-LSSmOrange. Cells were acquired using ImageStream (12H) and confocal microscopy (12I). NLRP3-mCitrine, green; ASC2-LSSmOrange, yellow; ASC-mPlum, red; DAPI, blue. Arrowheads, ASC specks. Scale bars, 10 μm (12H) and 20 μm (12I). (12J) Immunoblot showing analysis of cell lysates of mouse immortalized macrophages (IMACs) stably expressing LSSmOrange (control), bat or human ASC2, or Hupa4 ASC2-LSSmOrange, primed with LPS for 3 h. (12K) Histogram showing results by flow cytometry of sorted mouse immortalized macrophages (iMACs) stably expressing LSSmOrange (control), bat or human ASC2, or Hupa4 ASC2-LSSmOrange. (12L) Immunoblot showing analysis of cell lysates of THP-1 macrophages stably expressing mCitrine (control), bat or human ASC2 or Hupa4 ASC2-mCitrine, primed with LPS for 3 h. (12M) Histogram showing results by ImageStream of THP-1 macrophages stably expressing mCitrine (control), bat or human ASC2 or Hupa4 ASC2-mCitrine. Data are presented as mean±s.d. of three independent experiments (12B-12G), or as representative of three independent experiments (12H-12M). *P<0.05, **P<0.01, ****P<0.0001, NS, not significant; one-way ANOVA with Bonferroni's multiple comparisons test (12B-12G).

FIG. 13. Table showing a summary of the presence (+) or absence (−) of ASC2 gene locus in the genomes of representative marsupial (Metatheria) and placental (Eutheria) mammals. Highlighted boxes, Chiroptera (bats) and Primates.

FIG. 14. Table showing ASC2 sequences used in the evolutionary analysis. These sequences were retrieved from NCBI databases. Phascolarctos cinereus was included as an outgroup.

FIG. 15. Table showing ASC sequences used in the evolutionary analysis. These sequences were retrieved from NCBI databases. Phascolarctos cinereus was included as an outgroup.

FIG. 16. Schematic diagram showing the ASC inhibitor drug screening assay using the NanoBIT system. Upon PYD-PYD protein-protein interaction, LgBiT and SmBiT complement and release a luminescence signal when the substrate, furimazine, is added.

FIGS. 17A-17C. Bar charts showing relative luciferase unit (RLU) of HEK293T cells transfected with a pair of plasmids containing human ASC PYD fused with NanoBiT tags at 100 ng (17A), 10 ng (17B), or 1 ng (17C) each. Total DNA transfected for each condition was 200 ng. Conditions with lower amounts of LgBIT/SmBiT-tagged PYD plasmids were topped up to 200 ng DNA with an empty plasmid without inserts. PYD* denotes PYD containing E13R, K21E/K22E, and F59E mutations. Lg and Sm denote LgBiT and SmBiT, respectively. Data are representative of three independent experiments. Individual data points represent technical triplicates.

FIG. 18. Bar chart showing signal-to-noise ratio (S/N ratio) of HEK293T cells transfected with a pair of plasmids containing human ASC PYD fused with NanoBiT tags at 100, 10, or 1 ng each. Total DNA transfected for each condition was 200 ng. Conditions with lower amounts of LgBIT/SmBIT-tagged PYD plasmids were topped up to 200 ng DNA with an empty plasmid. Lg and Sm denote LgBIT and SmBIT, respectively. Data are representative of three independent experiments. A ratio higher than the dotted line y=1 indicates more signal than noise.

FIGS. 19A-19B. Bar charts showing relative luciferase unit (RLU) of HEK293T cells transfected with LgBiT/SmBiT-tagged PYD pairs at 10 ng (19A) or 1 ng (19B) each and bat ASC2-LSSmOrange at 0 or 10 ng. Total DNA transfected for each condition was 220 ng. Conditions with lower amounts of LgBiT/SmBiT-tagged PYD plasmids were topped up to 220 ng DNA with an empty plasmid. PYD* denotes PYD mutant containing E13R, K21E/K22E, and F59E mutations. Lg and Sm denote LgBiT and SmBIT, respectively. Data are representative of three independent experiments. Individual data points represent technical triplicates.

FIGS. 20A-20B. Bar charts showing percent inhibition of HEK293T cells transfected with LgBoT/SmBiT-tagged PYD pairs at 10 ng (20A) or 1 ng (20B) each and bat ASC2-LSSmOrange at 0 or 10 ng. Total DNA transfected for each condition was 220 ng. Conditions with lower amounts of LgBIT/SmBiT-tagged PYD plasmids were topped up to 220 ng DNA with an empty plasmid. Percentage inhibition was calculated as inhibition (%)=(wild-type PYD pair without ASC2-wild-type PYD pair with ASC2)/(wild-type PYD pair without ASC2-PYD mutant pair). Lg and Sm denote LgBiT and SmBIT, respectively. Data are representative of three independent experiments.

FIG. 21. TAT-hupa4 is delivered into PBMCs. Immunoblot analysis showing TAT-His and TAT-Hupa4-His proteins in cell lysates of PBMCs, delivered with TAT-His or TAT-Hupa4-His (TAT-HuPa4) proteins at the indicated concentrations.

FIGS. 22A-22C. TAT-hupa4 inhibits NLRP3 inflammasome activation by nigericin in human peripheral blood mononuclear cells (PBMCs). (22A) Immunoblot analysis showing TAT-His and TAT-hupa4-His proteins in cell lysates of PBMCs primed with LPS for 3 h, delivered with TAT-His or TAT-hupa4-His (TAT-huPa4) proteins at indicated concentrations for 1 h, washed, and then stimulated by nigericin (Nig) for 1 h. (22B and 22C) Bar charts showing LDH release (22B) and secretion of IL-1β by ELISA (22C) in cell-free supernatants of PBMCs primed with LPS for 3 h, delivered with TAT-His or TAT-Hupa-His (TAT-hupa4) proteins at indicated concentrations for 1 h, washed, and then stimulated by nigericin for 1 h. Data are presented as mean±s.d. of four independent experiments (22B and 22C). **P<0.01, ***P<0.001, ****P<0.0001, NS=non-significant, by one-way ANOVA with Bonferroni's multiple comparisons test.

FIG. 23. TAT-hupa4 inhibits inflammasome activation induced by α-synuclein in human peripheral blood mononuclear cells (PBMCs). Bar charts showing secretion of IL-1β by ELISA in cell-free supernatants of PBMCs delivered with TAT-His or TAT-Hupa4-His (TAT-Hupa4) proteins at indicated concentrations for 1 h, washed, and then stimulated by α-synuclein for 24 h. Data are presented as representative mean±s.d. of three independent experiments. **P<0.01, ns=non-significant, by one-way ANOVA with Bonferroni's multiple comparisons test.

FIGS. 24A-24B. TAT-hupa4 inhibits NLRP1 inflammasome activation by talabostat in human epidermal keratinocytes, adult (HEKa). (24A) Immunoblot analysis showing TAT-His and TAT-hupa4-His (TAT-huPa4) proteins in cell lysates of HEKa stimulated by talabostat for 8 h, delivered with TAT-His or TAT-hupa4-His proteins at indicated concentrations for 1 h, washed, and then stimulated by talabostat for another 15 h. (24B) Bar charts showing secretion of IL-1β by ELISA in cell-free supernatants of HEKa stimulated by talabostat for 8 h, delivered with TAT-His or TAT-hupa4-His (TAT-hupa4) proteins at indicated concentrations for 1 h, washed, and then stimulated by talabostat for another 15 h. Data are presented as representative of three independent experiments (24B). ***P<0.001, ****P<0.0001, ns=non-significant, by one-way ANOVA with Bonferroni's multiple comparisons test.

FIGS. 25A-25C. Bat ASC2 inhibits talabostat-induced NLRP1 inflammasome activation in N/TERT-1 cells. (25A) Histogram by flow cytometry showing sorted immortalized N/TERT-1 keratinocytes stably expressing mCitrine alone (control), bat or human ASC2 tagged with mCitrine. (25B) LDH release by LDH release assay in cell-free supernatants of sorted N/TERT-1 stably expressing mCitrine alone (control), bat or human ASC2 tagged with mCitrine, treated with different concentrations (0, 20, 200 μM) of talabostat for 24 h. (25C) IL-1β secretion by ELISA in cell-free supernatants of sorted N/TERT-1 stably expressing mCitrine alone (control), bat or human ASC2 tagged with mCitrine, treated with different concentrations (0, 20, 200 μM) of talabostat for 24 h. Data are presented as mean±s.d. of three biological replicates. *P<0.05, **P<0.01, ***P<0.001, ****P<0.0001, ns, not significant, by two-way ANOVA with Bonferroni's multiple comparison test (25B and 25C).

FIGS. 26A-26B. Bat and hupa4 ASC2 interacts with ASC at higher affinity than human ASC2. Immunoprecipitation of ASC-Flag with anti-Flag antibody from HEK293T cells transfected with human (26A) or bat (26B) ASC-Flag and His alone (control), bat, human, or Hupa4 ASC2-His, followed by immunoblot analysis alongside total cell lysates (TCL). 3 μg human ASC2, in contrast to 0.25 μg control, bat or hupa4 ASC2, were transfected to normalize the expression. The TCL and immunoprecipitates were immunoblotted with anti-Flag (top) or anti-His (bottom) antibodies. Data are representative of three independent experiments (26A and 26B).

FIGS. 27A-27B. ASC2 of non-primate and non-bat species shows variable function, from inhibitory, neutral to activating effect on inflammasome activation. (27A) Bar chart showing quantification of ASC speck formation by flow cytometry in HEK293T cells transfected without (negative control) or with human NLRP3-mCitrine, human ASC-mPlum, and LSSmOrange alone (control), bat ASC2, human ASC2, Hupa4 ASC2 or ASC2 of 16 non-primate non-bat species tagged with LSSmOrange. These 16 species are grouped into reptiles, marsupials and two groups of placental mammals, including group I from Proboscidea, Pilosa and Cingulata order and group II from Dermoptera, Scandentia and Rodentia order. (27B) Bar chart showing mean fluorescent intensity of LSSmOrange by flow cytometry in HEK293T cells transfected without (negative control) or with human NLRP3-mCitrine, human ASC-mPlum, and LSSmOrange alone (control), or bat, human, Hupa4, or ASC2 of 16 non-primate and non-bat species tagged with LSSmOrange. Data are presented as mean±s.d. of two independent experiments (27A and 27B). ****P<0.0001, ns, not significant, by one-way ANOVA with Bonferroni's multiple comparison test.

### EXAMPLES

In the following experimental examples, the inventors demonstrate that endogenous bat ASC2 is a potent negative regulator of the inflammasome in bats, and moreover potently inhibits human and mouse inflammasome function in vitro, and mouse inflammasome activity in vivo.

The inventors demonstrate that bat ASC2 behaves as a competitive inhibitor of ASC, and generate a potent inflammasome inhibitor based on human ASC2 through substitution of four amino acid positions with the corresponding residues found in bat homologues of ASC2.

The inventors further demonstrate that bat ASC2 ameliorate peritonitis induced by MSU crystals (the causative agent of gout), and that bat ASC2 suppresses the inflammatory response induced by several different viruses (IAV, ZIKV and PRV3M) in vitro.

It is also shown that fusion of bat ASC2 to the cell-penetrating peptide TAT facilitates intracellular delivery to human macrophages.

### Example 1: Materials and Methods for Examples 2-6

Reagents. LPS-B5 Ultrapure (tlrl-pb5lps), nigericin (tlrl-nig), FLA-PA Ultrapure (tlrl-pafla), Monosodium Urate (MSU) Crystals (tlr-msu), Poly(dA:dT) (tlrl-patn), phorbol 12-myristate 13-acetate (PMA) (tlrl-pma), Hygromycin B Gold (ant-hg-1), Primocin (ant-pm-1), and Pam3CSK4 (tlrl-pms) were purchased from InvivoGen. α1-2,4,6 fucosidase O was purchased from NEB. Recombinant mouse M-CSF protein (416-ML) was purchased from R&D Systems. FuGENE 6 (E2692) was from Promega. Lipofectamine 2000 (12566014) and 3000 (L3000015), anti-6X-His tag monoclonal antibody (MA1-135), nuclear stains (DAPI and propidium iodide), and LIVE/DEAD Fixable Blue and AQUA were obtained from ThermoFisher Scientific. Doxycycline hyclate (D9891) was obtained from Sigma-Aldrich. Alexa 488/647 Zenon labeling kits (Invitrogen) were used for direct labeling of antibodies for ImageStream and flow cytometry. Rabbit polyclonal anti-ASC (AL177) (human/mouse) was obtained from Adipogen. Monoclonal antibody to β-actin (A2228) was purchased from Sigma-Aldrich. Alexa Fluor® 488 anti-mouse IgG2a antibody, Alexa Fluor® 647 anti-mouse IgG2b antibody, PE/Dazzle™ 594 anti-mouse IgG (Poly4053), purified mouse IgG2a isotype control (MOPC-173), purified mouse IgG2b isotype control (27-35), purified anti-mouse CD16/32 antibody (Fc-block) (93), anti-CD11b (M1/70) BV421, anti-CD11b (M1/70) BV711, anti-CD11c (N418) APC, anti-Ly6C (HK1.4) BV510, anti-Ly6G (1A8) PE-Cy7, anti-CD19 (6D5) APC-Cy7, anti-NK1.1 (PK136) AF700, anti-Ter199 (TER-199) PE, anti-MHCII (M5/114.15.2) BV650, anti-CD3 (145-2C11) PerCP/BV605, Anti-CD8 (53-6.7) BV785 were purchased from Biolegend. Anti-CD44 (IM7) APC-H7 was purchased from eBiosciences. P. alecto ASC2-specific mAb (mouse IgG2a) and P. alecto ASC-specific mAb (mouse IgG2b) were generated by GeneScript's monoclonal antibody service. mPlum antibody from Origene was used to stain LSSmOrange. Anti-mouse/goat horseradish peroxidase (HRP)-conjugated secondary antibodies were from R&D Systems. Anti-SARS-COV-2 RBD monoclonal antibody CR3022 and biotinylated SARS-COV-2 RBD were purchased from GenScript. RosetteSep Human Monocyte Enrichment Kit was from STEMCELL. TAT-bat ASC2-His construct was generated by fusing HIV-1 TAT peptide sequence (YGRKKRRQRRR) with bat ASC2 followed by Xhol (LE) and 6×His tag on the C-terminus. The recombinant proteins were purified from E. Coli by ChinaPeptides. TAT-His peptide (TAT peptide+Xhol+6×His) was synthesized by GenScript.

Plasmids. Expression constructs for human NLRP3-mCitrine, bat NLRP3 (isoform 1)-mCitrine, human ASC-mPlum, all the ASC2-LSSmOrange or -mCitrine constructs, and their empty vectors were generated in the retroviral backbone of pQCXIH (Clontech). Plasmids encoding fusions with fluorescent tags generate proteins tagged on the C-terminus. Fusions with mCitrine tag were separated by Notl+1-aa (AAA) and those with mPlum or LSSmOrange tags were separated by Pacl+2-aa (LIN). Chimeras and site-directed mutagenesis of ASC2 were generated by overlapping extension PCR. Human ASC2, bat ASC2, hupa4 ASC2, and empty vector with 6×His tag were generated in pDual GC (Agilent Technologies). pVSV-G envelope vector for retroviral packaging was obtained from Clontech. All constructs were prepared with endotoxin-free plasmid maxi-prep kits (Omega Bio-tek).

Cells. All procedures for animal samples were compliant with all the relevant ethical regulations. Capturing and processing of P. alecto in Australia was approved by the Queensland Animal Science Precinct & University of Queensland Animal Ethics Committee (AEC #SVS/073/16/USGMS) and the Australian Animal Health Laboratory (AAHL) Animal Ethics Committee (AEC #1389 and AEC #1557). Processing of bats has been described previously 49. P. alecto and mouse splenocytes were isolated by grinding the spleen through a 100 μm cell strainer followed by red blood cell (RBC) lysis buffer (eBioscience). Mouse peripheral blood cells were isolated from submandibular veins with EDTA and lysed with RBC lysis buffer. Mouse bone marrows were harvested, processed and differentiated into BMDMs over 6 days of cultures using 10 ng/ml of mouse M-CSF recombinant proteins, 100 μg/ml Primocin (InvivoGen) with or without the presence of 1 μg/ml Doxycycline. Media was replaced on day 3 of each differentiation and experiments were performed on day 6. GP2-293 retroviral packaging cells were obtained from Clontech. BMDMs, Immortalized mouse macrophages (iMACs) and THP-1 cells were cultured in RPMI 1640 medium containing 10% FBS (Biological Industries). Vero, GP2-293, MDCK, PaKi and HEK293T cells were cultured in DMEM (Gibco) medium supplemented with 10% FBS. Sodium pyruvate and NEAA (Life Technologies) were supplemented into the culture media of GP-293 cells during retroviral packaging.

Generation of THP-1 and IMACs stable cells. GP2-293 cells, stably expressing gag and pol proteins, were co-transfected with pVSV-G envelope vector and LSSmOrange/mCitrine-only empty vector, bat or human ASC2 or hupa4 ASC2-LSSmOrange/mCitrine vector for 48 h. Supernatants containing retroviruses were collected, centrifuged at 500 g for 10 min and filtered through a 0.45 μm hydrophilic polyethersulfone filter (Millipore). THP-1 cells or iMACs were transduced with low MOI of retrovirus for 24 h to obtain a single viral copy per transduced cell. 72 h post-transduction, cells were selected by Hygromycin B antibiotic for at least 1 month to ensure stable expression. Single cell clones were generated for THP-1 cells stably expressing ASC2-LSSmOrange. THP-1 and iMacs stable cells were FACS-sorted for equal fluorescent intensity to ensure same expression levels across cell lines.

Inflammasome activation assays. BMDMs, THP-1 cells and iMACs were seeded at 1×10⁶/ml into 96-well plates. THP-1 cells were first differentiated for 16 h overnight with 100 ng/ml PMA. THP-1 macrophages were subsequently primed with 1 μg/ml LPS for 3 h. The medium was then removed and replaced with either with serum-free RPMI 1640 medium or media containing 6.7 μM nigericin for 120 min for LDH release assay and IL-1β detection by ELISA, or 30 min for detection of ASC specks by ImageStream. Primed THP-1 macrophages were also transfected with 1 μg/ml poly(dA:dT) using Lipofectamine 2000 for 4 hr or treated with 100 μg MSU crystals for 4 h in serum-free media. BMDMs were primed with 1 μg/ml LPS B5 with or without the presence of 1 μg/ml doxycycline for 3 h. The medium was then removed and replaced with either with serum-free medium or media containing 6.7 μM nigericin for 60 min for LDH release assay and IL-1β detection by ELISA, or 30 min for detection of ASC specks by ImageStream. Primed BMDMs were also transfected with 1 μg/ml poly(dA:dT) or 286 ng/ml flagellin using Lipofectamine 3000 for 4 h, or treated with 50 μg MSU for 16 h, in serum-free media with or without 1 μg/ml doxycycline. Primed iMACs were treated with 6.7 μM nigericin for 120 min. For ImageStream detection of ASC specks, HEK293T cells were seeded overnight into a 96-well plate and transfected with 12.5 ng human NLRP3-mCitrine, 1.5 ng of human ASC-mPlum, 50 ng indicated ASC2-LSSmOrange and 186 ng of empty vector using FuGENE 6. At 48 h post-transfection, cells were harvested by trypsinization and resuspended in FACS buffer for ImageStream analysis.

Viral infection of cells. Human H1N1 IAV strains A/NWS/33 (ATCC #VR-219) was propagated in Vero clone E6 cells in DMEM, 0.3% BSA, 25 mM HEPES and 1 μg/ml TPCK-treated trypsin. Zika virus strain Paraiba_01/2015 from Brazil (NCBI accession no. KX280026, gift from Pedro F. C. Vasconcelos at Instituto Evandro Chagas) and Pteropine orthoreovirus 3, PRV3M (commonly known as Melaka virus), were propagated in Vero clone B4 cells in 2% FBS DMEM. Both ZIKV and PRV3M were purified by ultracentrifugation in 20% sucrose overlaid at 125,000×g for 90 min. For IAV and PRV3M, BMDMs were primed with 1 μg/ml LPS-B5 for 3 h and then infected with IAV (MOI=1) for 2 h or PRV3M (MOI=5) for 4 h. For ZIKV, BMDMs were infected with ZIKV (MOI=1) for 2 h. Subsequently, the medium was replaced with AIM V serum-free medium (Invitrogen). At 24 h post-infection, cell free supernatants were collected for IL-1β detection by ELISA and virus titrations. 1 μg/ml of doxycycline was present throughout BMDMs infection. To titrate IAV, A/NWS/33, Madin-Darby canine kidney (MDCK, ATCC #CCL-34) cells were infected with IAV for 1 h at 37° C. After 1 h incubation, the medium was replaced with plaque medium (DMEM, 0.8% Avicel, 0.3% BSA, 25 mM HEPES and 1 μg/ml TPCK-treated trypsin). Plaques were fixed with 4% formaldehyde at 48 h post-infection and stained with 0.5% crystal violet. The viral titres were expressed in p.f.u./ml.

SARS-COV-2 immune complex-induced inflammasome activation. To generate afucosylated SARS-CoV-2 antibody, 2 units of α1-2,4,6 fucosidase O (NEB) is added to every 1 μM anti-SARS-COV-2 RBD monoclonal antibody CR3022 and incubated for 18 h at 50° C. To form SARS-COV-2 RBD-specific immune complex, 50 μl of 10 μg/ml biotinylated SARS-COV-2 RBD per well was first coated overnight on a 96-well high-affinity plate (Nunc) at 4° C. The plate was blocked with 10% FBS in DPBS for 1 h at room temperature, then 2 μg/ml CR3022 monoclonal antibodies were added and incubated for 1 h at 37° C. Monocytes were isolated from healthy donor blood using RosetteSep Human Monocyte Enrichment Kit (STEMCELL) per the manufacturer's instructions. For the priming experiment, monocytes treated with or without 50 μM MCC950 and primed with different concentrations of Pam3CSK4 (InvivoGen) (0, 1, and 100 ng/ml) were seeded at a density of 3×10⁶ cells per ml in RPMI 1640 medium containing 10% FBS (Gibco) into the plate containing SARS-COV-2 immune complex for 18 h at 37° C. For the TAT experiment, cells seeded at a density of 3×10⁶ cells per ml were treated with or without 8 μM TAT-His or TAT-bat ASC2-His for 1 h at 37° C. in a 5% CO2 incubator. Following treatment, monocytes were washed once with media and transferred to the plate containing 100 ng/ml Pam3CSK4 and SARS-COV-2 immune complex for 2 h at 37° C. Cell-free supernatants were collected for LDH release assay and IL-1β detection by ELISA.

Generation of bat ASC2/rtTA mice. Plasmid pTetO-batASC2-IRES-eGFP was generated. The target fragment was excised and purified and transgenic mice B6. Tg (TetO-bat ASC2-IRES-eGFP) were generated by pronuclear injection of that fragment into C57BL/6 wild-type embryos. The transgenic mouse was generated by Biological Resource Centre (BRC) at A*STAR. Five lines were initially analyzed, and subsequently a single line was used for most experiments. The progeny of Tg (batASC2) mouse lines were crossed with Tg (CAG-rtTA3) to generate Tg (batASC2/rtTA3) double transgenic mice. Single transgenic mice for bat ASC2 or CAG-rtTA3 were inter-crossed to maintain the mouse line. C57BL/6 wild-type mice were obtained from InVivos and Tg (CAG-rtTA3) mice were obtained from The Jackson Laboratory. Transgenic offspring were genotyped by polymerase chain reaction (PCR) using primers specific for CAG or bat ASC2. Briefly, ear clippings from offspring were lysed with DirectPCR (ViaGEN) and Protease K (Roche) according to manufacturer's instructions. PCR was performed on lysed samples using cycling conditions specific to each primer. Mice were housed in a specific pathogen-free animal facility and all experiments used age- and sex-matched mice of 10-14 weeks of age and were conducted according to procedure approved by the SingHealth Institutional Animal Care and Use Committee (IACUC).

Peritonitis induced by MSU crystals and ASC specks. 10- to 14-week-old age- and sex-matched WT and bat ASC2/rtTA mice were used for the experiments. Seven days prior to injection, mice were given water supplemented with 2 mg/ml doxycycline+5% sucrose. On the day of the challenge, mice were injected intraperitoneally with 200 μg of MSU crystal in 200 μl DPBS or 105 ASC speck particles in 200 μl PBS for 4 h. Mice were sacrificed and peritoneal lavage was performed by flushing the peritoneal cavity with 5 mL of ice-cold DPBS+2% FBS+2 mM EDTA with a 27 g needle. After gentle massaging, the lavage fluids were collected with a 25 g needle and a pasture pipette for the remaining fluid in the peritoneal cavity. The lavage fluids were centrifuged at 500×g for 5 min at 4° C. and cell-free lavage fluids were used for IL-1β detection by ELISA while the cell pellets were used for staining and analysis by flow cytometry.

In vivo viral infection. 10- to 14-week-old age- and sex-match WT and bat ASC2/rtTA mice were used for the viral infection study. One day prior to infection, mice were given water supplemented with 2 mg/ml doxycycline+5% sucrose. The following day, mice were anesthetized with isoflurane and were infected intranasally with 0.5×10⁵ p.f.u. IAV in 20 μl. Mice were monitored daily for up to 14 dpi for survival study. At day 3 post-infection, lungs of infected mice were dissected, weighed and homogenized using homogenizer in 1 ml of serum-free DMEM media. Cell debris was removed by centrifugation at 10,000×g for 5 min, and the virus-containing supernatants were used for titration and IL-1β detection by ELISA. The viral titres were quantified by plaque assay on MDCK cells as described above. The viral titres were expressed in p.f.u./g.

Production and purification of ASC specks. HEK293T cells stably expressing human ASC-mPlum was generated as described previously³⁸. Total cell lysates were prepared by lysis with CHAPS buffer (20 mM HEPES-KOH, PH 7.5, 5 mM MgCl₂, 0.5 mM EGTA, 0.1% CHAPS, supplemented with complete ULTRA protease inhibitor cocktail). The lysates were passed through a syringe with a 23 g needle for fifty times and centrifuged at maximum speed for 8 min at 4° C. Supernatants were incubated at 37° C. for 30 min to induce aggregation of ASC-mPlum. ASC-mPlum specks were subsequently FACS-sorted into PBS.

LDH release assay. Cytotoxicity induced by various stimuli was assessed by the release of cytosolic LDH. Cell culture media was collected and centrifuged at 500 g for 5 min at 4° C. and LDH release was quantified in the cell-free supernatants using Cytotoxicity Detection Kit PLUS (Roche). The kit was used according to the manufacturer's instructions. The percentage of LDH release was calculated as LDH release (%)=(experiment value-Negative control)/(Positive control-Negative control)×100. Negative control is defined as the baseline LDH release from untreated cells, while positive control is the maximum LDH release from cells treated with lysis solution.

Immunoblot analysis. Cells were lysed in lysis buffer⁵⁰ containing complete ULTRA protease inhibitor cocktail (Roche). Proteins were separated by 6-15% SDS-PAGE gels, transferred onto a 0.45 μm polyvinylidene difluoride (PVDF) membrane with a Trans-Blot Turbo transfer system (Biorad). Transferred membranes were blocked with 5% BSA in TBS-T (0.1% Tween-20) for 1 h at room temperature and incubated overnight with specific primary antibodies in 5% BSA in TBS-T in a cold room. The following day, membranes were incubated with HRP-conjugated secondary antibodies for 1 h at room temperature. Membranes were developed using Amersham ECL Prime Western blotting detection reagent (GE Healthcare) and signals were detected with a ChemiDoc MP Imaging System (Bio-Rad). Coomassie staining was performed using PageBlue Protein Staining Solution (Thermo).

ELISAs. Human and mouse IL-1β in cell-free supernatants, lavages or homogenates were quantified by ELISA according to the manufacturer's instructions (R&D Systems; DY201 and DY401). Filtered 1% BSA in PBS was used as the blocking buffer and assay diluent. 3,3′,5,5′-tetramethylbenzidine (TMB) chromogen solution (Invitrogen) and TMB stop solution (VWR) were used in the assay. Absorbance was read using Cytation 5 imaging reader (BioTek).

ImageStream imaging flow cytometry. Cell supernatants were transferred to a 96-well V-bottom plate and centrifuged at 500×g for 5 min at 4° C. THP-1 macrophages and BMDMs were incubated with ice-cold 5 mM EDTA in DPBS for 10 min at RT, followed by pipetting to detach the cells. Detached cells were combined with cell pellets from the supernatants in the V-bottom plate and centrifuged again. Cell pellets were resuspended in 4% paraformaldehyde in TBS and permeabilized with 0.3% Triton X-100+2% FBS+1% NGS (normal goat serum) in TBS for 15 min at 4° C. Cells were washed twice in wash buffer (TBS+2% FBS) and stained with primary ASC antibody pre-labeled with Zenon labeling kit for 1 h in TBS+2% FBS+1% NGS. Cells were washed twice after antibody staining and were resuspended in FACS buffer (DPBS+5% FBS+5 mM EDTA). Nuclei were stained with DAPI. For reconstituted HEK293T cells, cells were harvested by detaching with trypsin and cell pellets were resuspended directly in FACS buffer. Cells were acquired with ×40 magnification using INSPIRE software on an Amnis ImageStreamX Mk II imaging flow cytometer. A minimum of 10,000 single cells were acquired for each sample and analyzed with the inbuilt IDEAS software. First, cells in focus were gated using the bright-field r.m.s values. Single cells were then selected with an intermediate bright-field area and a high aspect ratio. Sub-Go/G1 cells were excluded based on the DAPI intensity. Similar fluorescent intensity for mCitrine, mPlum and LSSmOrange for HEK293T cells or LSSmOrange for THP-1 was gated. Cells with a small area and a high maximum pixel value of ASC signal and ASC2 signal were gated as ASC speck-positive and ASC2 filament-positive cells, respectively.

Flow cytometry. Fluorophore-conjugated antibodies that were used are listed in the reagents section. Harvested mouse cells were blocked with 1% mouse Fc block (purified anti-mouse CD16/32, Biolegend) for 15 min at 4° C. and stained with surface marker antibodies in FACS buffer (PBS supplemented with 2% FBS) for 30 min at 4° C. in the dark. For experiments where Fixable LIVE/DEAD Blue was not used, DAPI was added 10 minutes prior to acquisition on flow cytometer. Thawed P. alecto splenocytes were blocked with 10% FCS for 15 minutes at 4° C. before staining for 30 minutes at 4° C. with anti-CD44, anti-CD11b and fixable viability dye. Intracellular staining was then performed with BD Cytofix/Cytoperm kit. Cells were fixed and permeabilized for 30 minutes at 4° C., before incubating with hybridoma supernatant of anti-bat ASC and anti-bat ASC2 antibodies for 30 minutes at 4° C. The cells were then stained with goat-anti-mouse IgGs conjugated with PE/Dazzle™ 594 for 30 minutes and washed thrice before acquisition on the flow analyser. Fluorescence Minus One (FMO) controls were set up and defined as the sample incubated with all the antibodies minus one control. Isotype controls for IgG2b and IgG2a were also included for anti-bat ASC and anti-bat ASC2 staining respectively. Acquisition was done on LSRFortessa flow cytometer (BD Biosciences) equipped with 355 nm, 405 nm, 488 nm, 561 nm and 640 nm lasers. Compensation beads (Invitrogen, BD Biosciences, and Beckman Coulter) or stained cells were used to set up compensation calculations on BD FACSDiva software v 8.0.1. Acquired cells were analyzed using Flowjo software v.10 (TreeStar).

Confocal imaging. For detection of ASC specks, HEK293T cells were seeded onto glass coverslips (13 mm in diameter) overnight and transfected with 12.5 ng human NLRP3-mCitrine, 1.5 ng human ASC-mPlum, 50 ng indicated ASC2-LSSmOrange and 186 ng of empty vector using FuGENE 6. At 48 h post-transfection, cells were fixed with 10% formalin for 15 min at room temperature. DAPI (1:2000 dilution) was used for nuclear staining. All samples were then mounted and examined using the Leica TCS SP8 confocal microscope. For observation of ASC-ASC2 interaction in bat cells, thawed bat PBMCs were allowed to recover in RPMI 1640 medium containing 10% FBS for 2 h at 37° C. Cells were then centrifuged at 500 g for 5 min, resuspended, and incubated in FACS buffer supplemented with anti-CD11b (M1/70) BV421 antibody (1:50 dilution) for 10 min at 37° C. Thereafter, cells were centrifuged again, resuspended in a fresh medium, and seeded onto poly-L-lysine-coated glass coverslips. At 0.5 h post-seeding, cells were fixed with 10% formalin for 15 min, permeabilized with 0.3% Triton X-100 in PBS for 15 min, and then blocked with 10% normal goat serum for 1 h at room temperature. Subsequently, cells were incubated with primary ASC and ASC2 antibodies that were diluted in 5% normal goat serum at 4° C. overnight followed by secondary antibodies diluted in PBS for 1.5 hours at room temperature. Propidium iodide (1:200 dilution) was used for nuclear staining. All samples were then mounted and examined using the Leica TCS SP8 confocal microscope.

qPCR. HEK293T and PaKi cells were seeded overnight in 12-well plates and transfected with 0.5 μg of empty vector, bat or human ASC2 or hupa4 ASC2 tagged with His in pDual GC vectors using FuGENE 6 (HEK293T) and Lipofectamine 3000 (PaKi). At 48 h post-transfection, RNA was extracted from the cells using Total RNA Kit (Omega) according to manufacturer's instructions. RNA was converted to cDNA using the QuantiTect reverse transcription kit (Qiagen). qPCR reactions were prepared with SensiFAST SYBR No-ROX kit (Bioline) and qPCR was performed on CFX96 Touch Real-Time PCR Detection System (Bio-Rad) using the following cycling conditions: 95° C. for 5 min, 40 cycles of 95° C. for 10 s and 55° C. for 30 s, with a melt curve cycle. The mRNA levels were quantified by primers targeting the shared C-terminal tag-encoding region of pDual GC vectors (Forward: TCAGAAGAAGATCTGGAACAGAAG; Reverse: AGTGATGGTGATGGTGATGTC) and were normalized to housekeeping gene GAPDH.

Read-mapping analysis. Illumina HiSeq RNA-seq sequencing was performed on both CD11b+MHC-II− (putative monocytes/macrophages) and CD11b+MHC-II+ (putative dendritic cells) of P. alecto splenocytes (n=5) as previously described⁵¹. Bowtie was used to map the RNA-seq reads to the coding region of P. alecto ASC and ASC2. The mapped read depth was then extracted for each nucleotide position.

TAT-ASC2 Delivery. THP-1 cells were seeded at 1×10⁶/ml into 96-well plates and were differentiated for 16 h overnight with 100 ng/ml PMA. The following day, cells were primed with 1 μg/ml LPS for 3 h followed by delivery of different concentration of TAT-His or TAT-bat ASC2-His (1 μg, 5 μg and 10 μg) for 1 h in 50 μl PBS+10% glycerol. Subsequently, 50 μl of serum free RPMI 1640 medium containing 13.4 μM nigericin was added to the cells post TAT peptide delivery for 2 h. Cell free supernatants were collected for LDH release assay and IL-1β detection by ELISA and cells were harvested for immunoblot analysis.

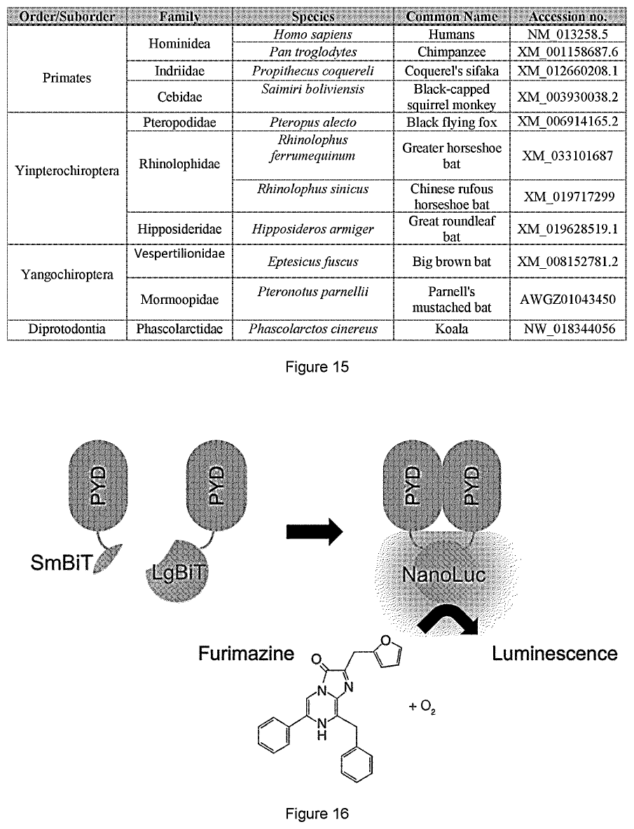

Evolutionary studies. ASC and ASC2 orthologs from four primates (Homo sapiens, Pan troglodytes, Propithecus coquereli and Saimiri boliviensis), six bats including four from Yinpterochiroptera and two from Yangochiroptera (P. alecto, Rhinolophus ferrumequinum, Rhinolophus sinicus, Hipposideros armiger, Eptesicus fuscus, Pteronotus parnellii) and an ungulate (Delphinapterus leucas) were retrieved from NCBI databases (as of 23/03/20) or identified from genomes by discontiguous MegaBLAST (BLAST+2.7.1) with max e-value of 1e-5 and word size of 11 when P. alecto genomic sequence served as query. Source of sequences are detailed in FIG. 14 and FIG. 15. ASC for R. ferrumequinum was removed due to poor sequence quality at its locus. Sequence alignment was generated by MAFFT (v7.450)⁵² and was used to plot the phylogenetic tree by Maximum-Likelihood method with General-Time-Reversible (GTR) model and 1,000 Bootstrap replicates in PHYML software (v3.3.20180621) 53. MegaBLAST search using full-length human ASC2 sequence was performed in the genomes of representative marsupial and placental mammals from NCBI databases (as of 23/03/20).

Mass spectrometry. CR3022 antibody underwent IdeS treatment before LCMS analysis. Briefly, 100 μl of deionized water was added to 5000 units of lyophilized IdeS Protease (Promega) to make a 50 unit/μl solution. 21 μg of CR3022 antibody was treated with 50 units of IdeS at 37° C. for 45 mins. The reaction was quenched with formic acid at a final concentration of 0.1%. The LCMS analysis was done on a Q Exactive Plus Biopharma system coupled with Vanquish UHPLC (Thermo Fisher Scientific, Bremen, Germany). The spectra recorded was analyzed by the Thermo BioPharma Finder software. The control and treated samples were analyzed on a 10 cm Thermo Scientific MAbPac column using 10 min gradient.

The following experimental parameters were used on the Orbitrap platform: the instrument was calibrated externally using Thermo Scientific Pierce Calibration Solutions, the source was operated in positive mode, the capillary voltage was set to 3.8 kV, the capillary temperature was 320° C., and the protein mode was on with pressure at 0.2. MS spectra were recorded at a resolution of 140,000.

Statistical analysis. Data are presented as mean and s.d. of multiple biological replicates or independent experiments as indicated. Statistical analysis was performed using GraphPad Prism software. Results were tested for significance using two-tailed unpaired Student's t-tests when two conditions were compared. One-way and two-way ANOVA involving one and two independent variables, respectively, with Bonferroni's multiple comparisons test, were performed if more than two samples or conditions were compared. Survival curves were generated via the product-limit method of Kaplan and Meier and comparisons were made via the log-rank test. Data were considered significant if *P<0.05, ** P<0.01, ***P<0.001, ****P<0.0001.

### Example 2: Bat ASC2 Blocks the Human and Mouse Inflammasomes

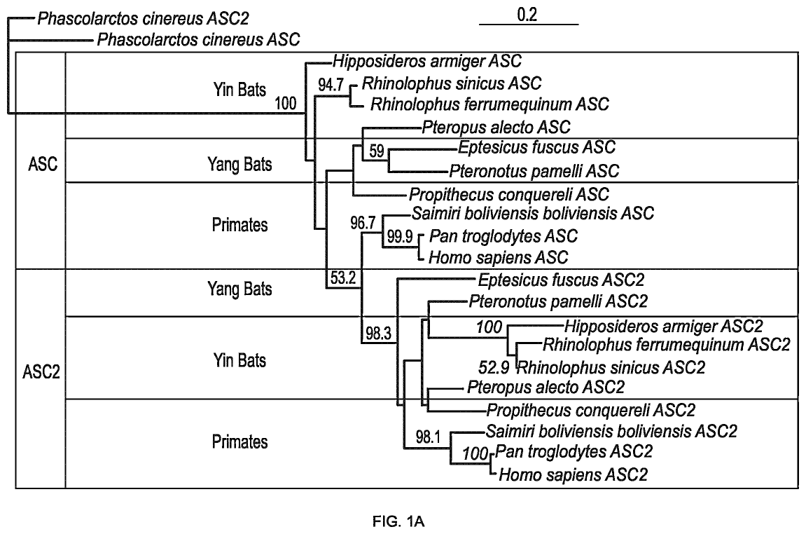

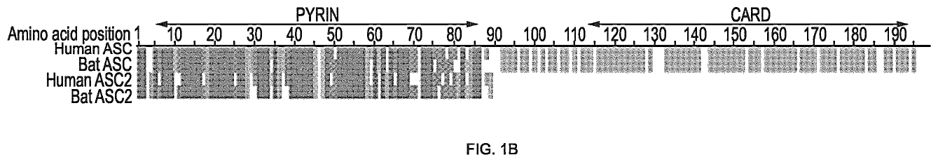

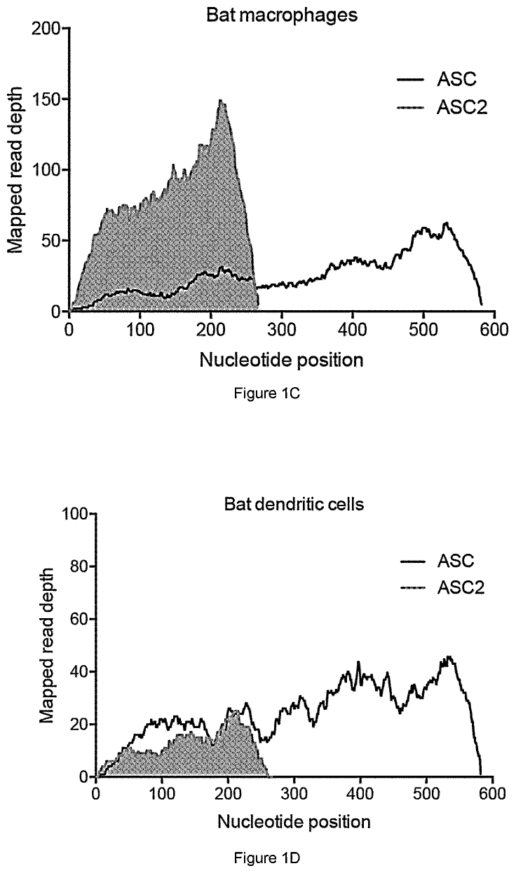

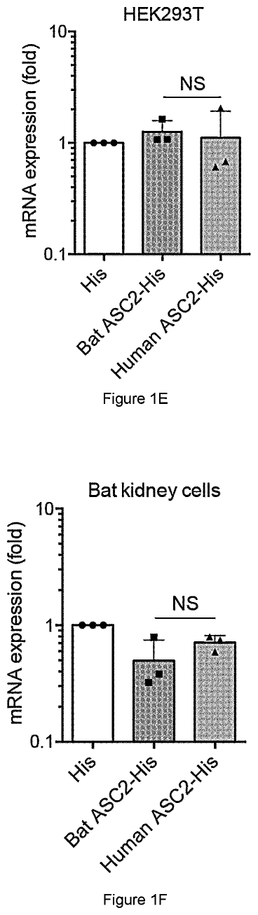

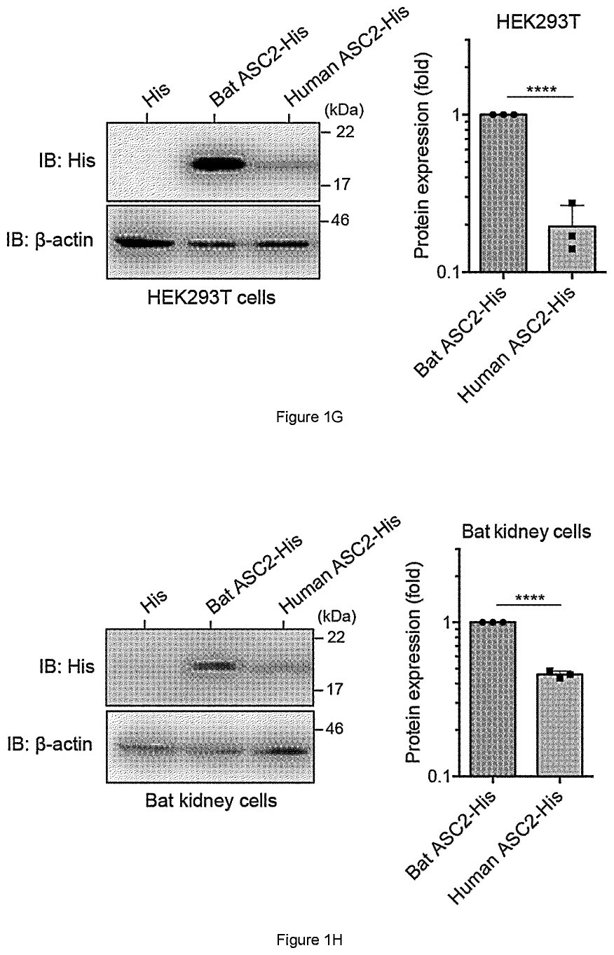

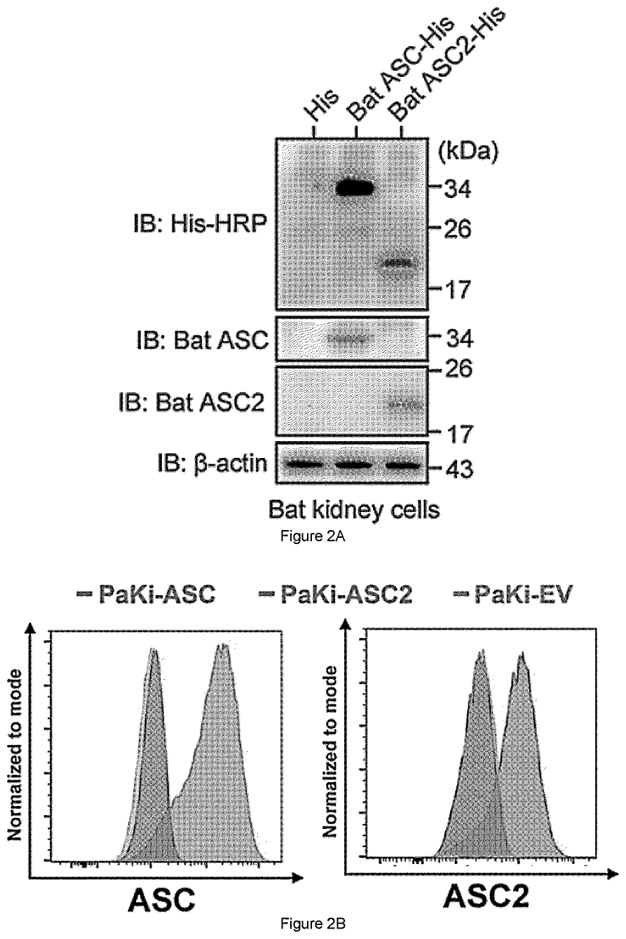

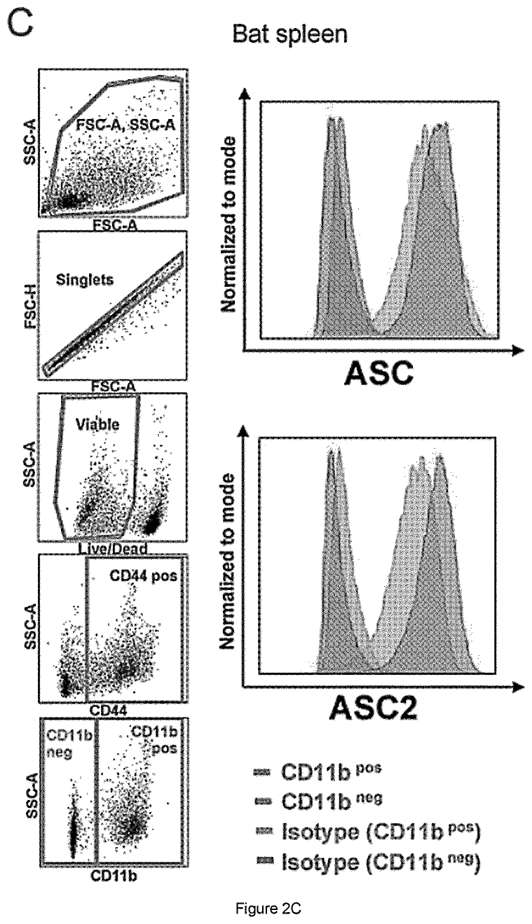

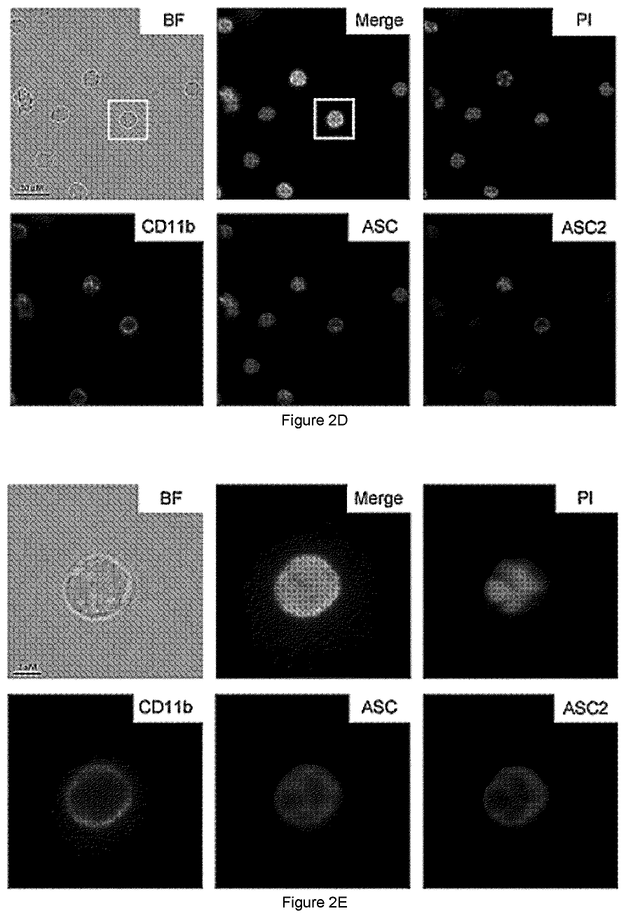

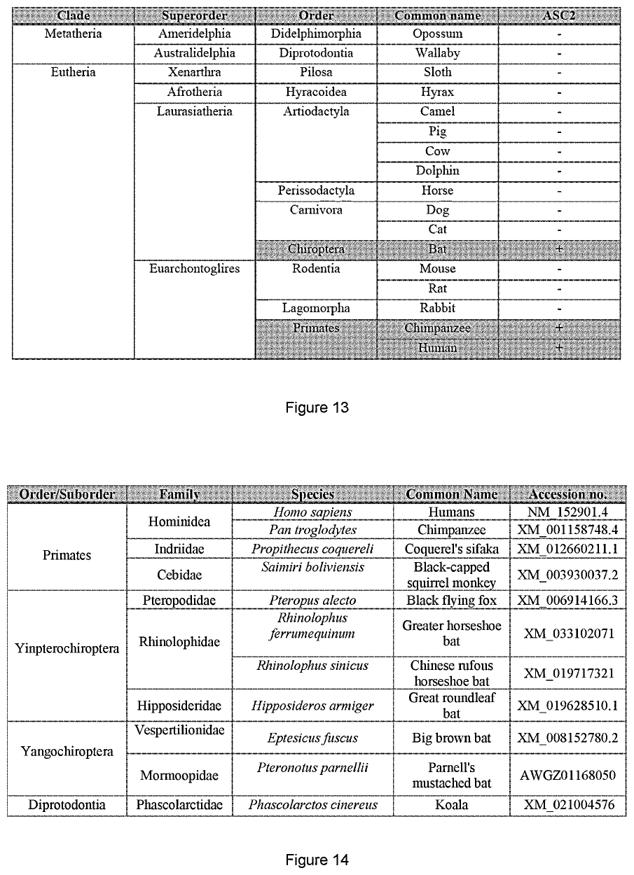

To examine the presence of ASC2 at the genomic level across mammalian species, a MegaBLAST search using full-length human ASC2 sequence was performed in the genomes of representative marsupial and placental mammals. In contrast to the previous report indicating ASC2 is found exclusively in primates⁸, ASC2 was identified in all the six bat species examined from both Yinpterochiroptera and Yangochiroptera suborders (FIG. 1A, FIG. 13 and FIG. 14). The amino acid sequence alignment between ASC2 and ASC (also known as PYCARD) for human and bat (Pteropus alecto) showed the presence of the PYRIN domain and the lack of CARD domain for ASC2 (FIG. 1B). The phylogenetic analysis of ASC2 and ASC for 4 primates and 6 bat species indicated a shared common ancestor for primates and bat ASC2 that possibly originated from ASC, rather than independent evolutionary events (FIG. 1A, FIG. 14 and FIG. 15). While ASC2 is not detectable at the mRNA level in most human tissues including the spleen⁹, read-mapping analysis using RNA-seq reads from bat splenocyte subsets showed ASC2 was highly expressed relative to ASC in the bat putative monocytes/macrophages (CD11b⁺MHC-II⁻) but similar of slightly lower in dendritic cells (CD11b⁺MHC-II⁺) (FIG. 1C and FIG. 1D). As ASC was previously shown to constitutively express in bat tissues similar to that in human or mouse tissues³⁸, this result suggests a high constitutive expression of ASC2 in bat immune cells. Using flow cytometric analysis with validated monoclonal antibodies specifically raised against P. alecto ASC2 and ASC proteins (FIG. 2A and FIG. 2B), a higher percentage of ASC2-expressing cells than ASC-expressing cells, and co-expression of ASC2 and ASC in ASC-positive cells were observed in CD11b⁺ myeloid cells of bat splenocytes (FIG. 2C). To further assess the protein expression, human and bat ASC2 fused to His tag were overexpressed side by side in both human embryonic kidney (HEK293T) cells and P. alecto kidney (PaKi) cells. Despite a similar mRNA level (FIG. 1E and FIG. 1F), a higher protein level was observed for bat ASC2 compared to human ASC2 in both cell lines (FIG. 1G and FIG. 1H), suggesting a post-transcriptional mechanism of increased protein expression for bat ASC2. Altogether, bat ASC2 is highly expressed endogenously and experimentally when overexpressed at both mRNA and protein levels.

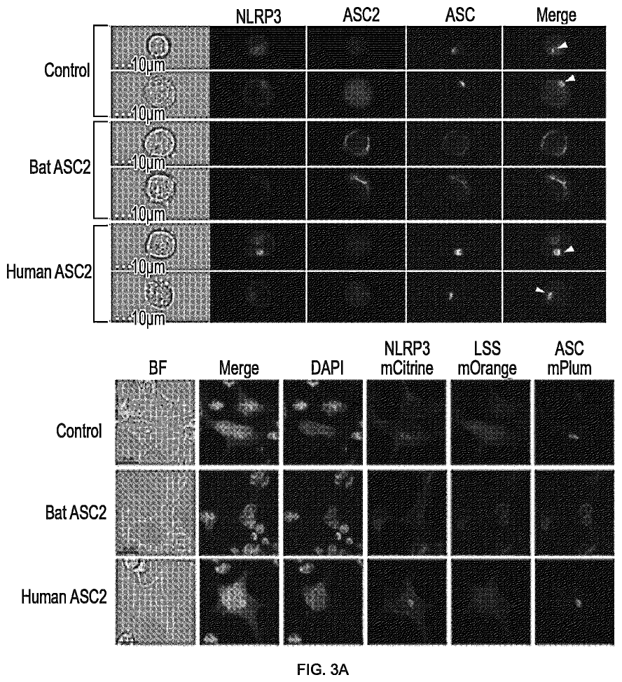

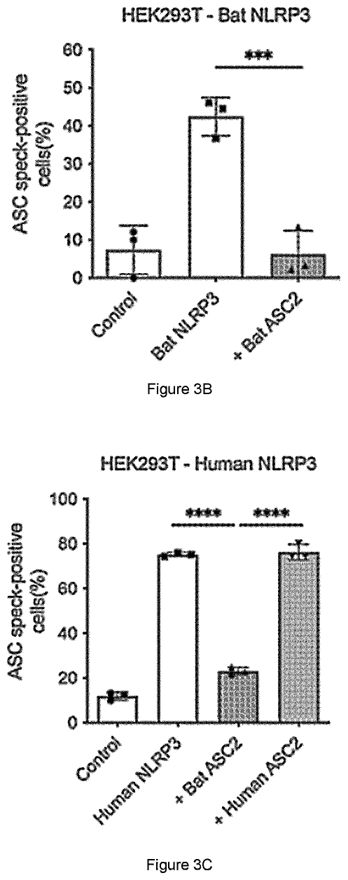

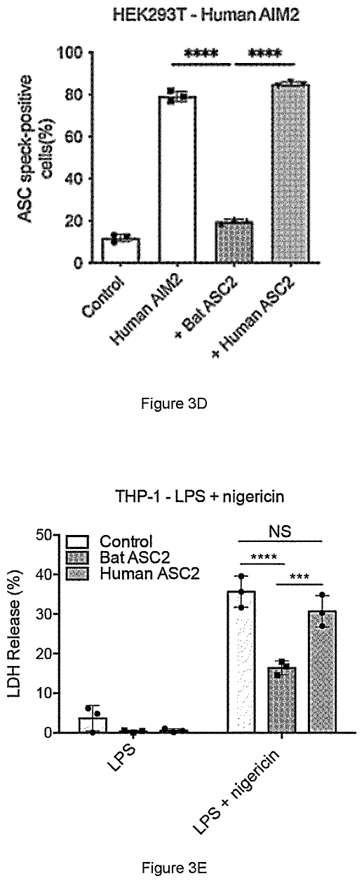

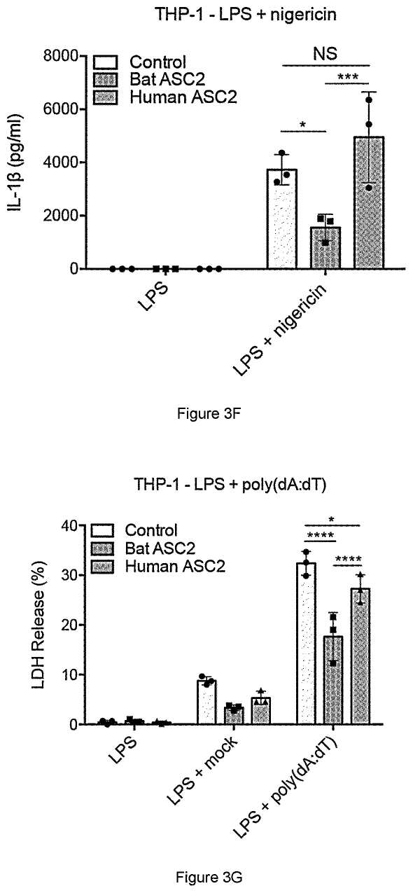

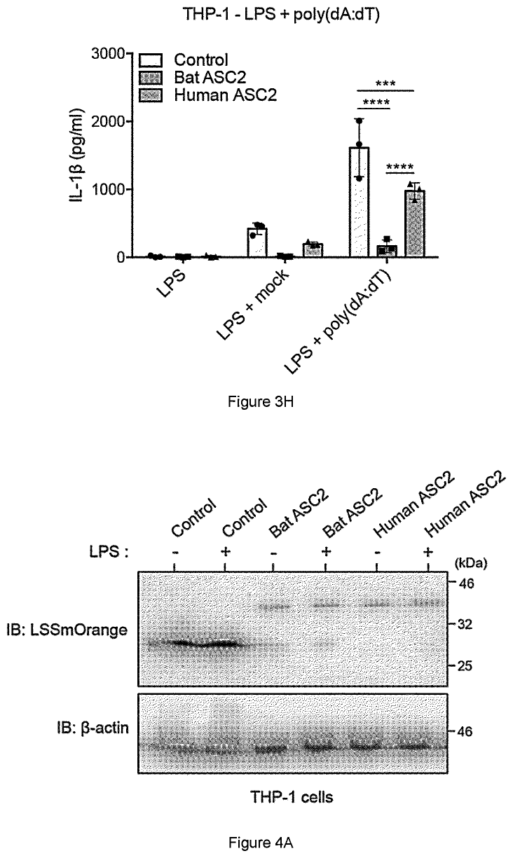

These observations of high expression of bat ASC2 led to further investigation into its function. In human HEK293T cell lines reconstituted with bat or human NLRP-3-mCitirne or AIM2-mCitirine and ASC-mPlum, overexpression of bat ASC2 not only inhibited bat NLRP3 inflammasome (FIG. 3B), but also suppressed the activation of human NLRP3 (FIG. 3A and FIG. 3C) and human AIM2 inflammasomes (FIG. 3D), indicated by the reduction of ASC speck formation analyzed with ImageStream imaging flow cytometer. In contrast to diffusely distributed human ASC2 protein, bat ASC2 formed fine and long filaments and disrupted the formation of ASC specks (FIG. 3A). A co-localization between bat ASC2 and human ASC, but not between human ASC2 and human ASC, was observed, suggesting a stronger interaction (FIG. 3A, FIG. 2D and FIG. 2E). Importantly, bat ASC2 was significantly more potent in inhibiting human NLRP3 and AIM2 inflammasomes than human ASC2, after normalizing the expression level by gating the equal signal intensity using ImageStream (FIG. 3C and FIG. 3D).

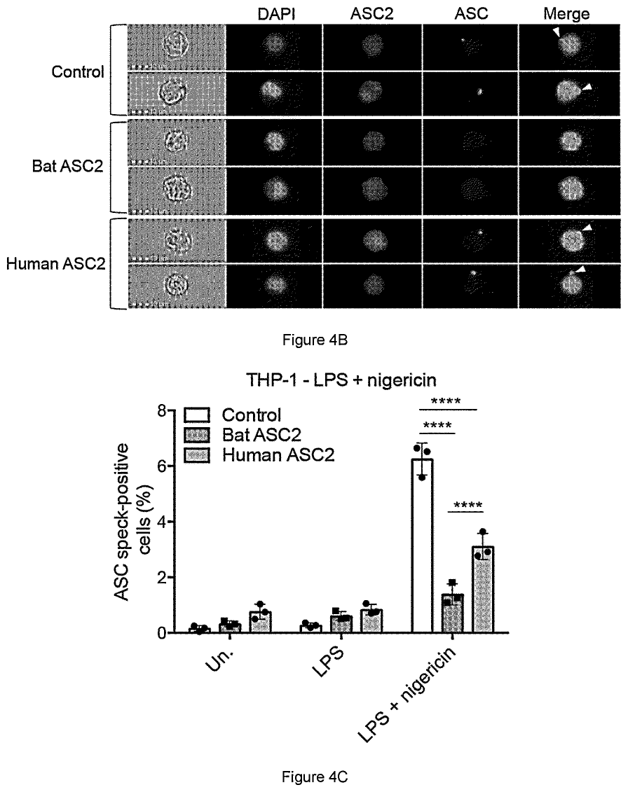

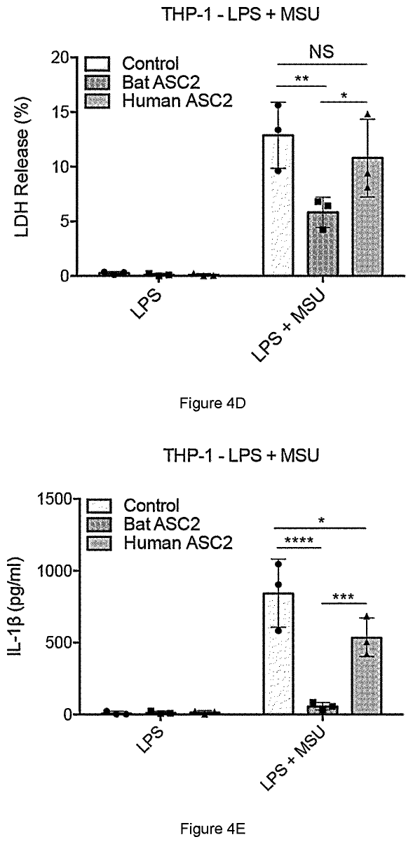

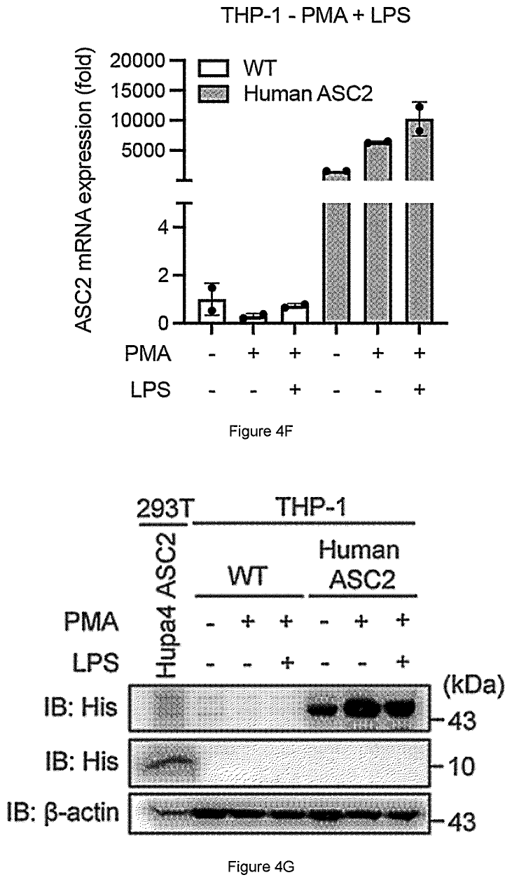

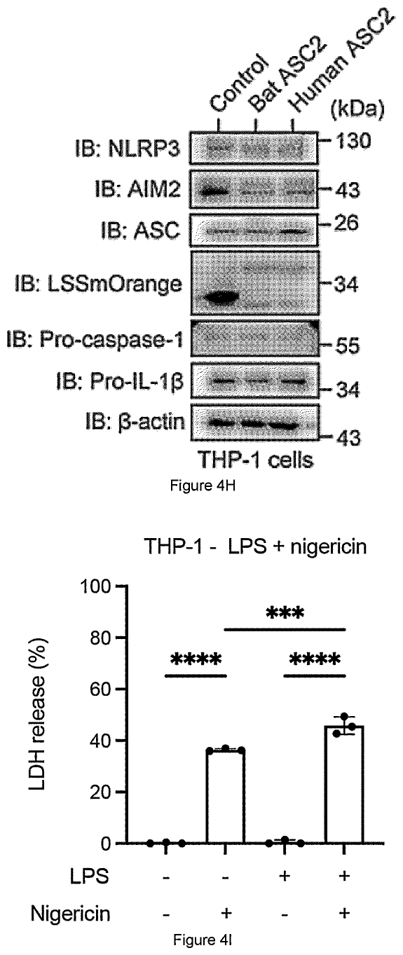

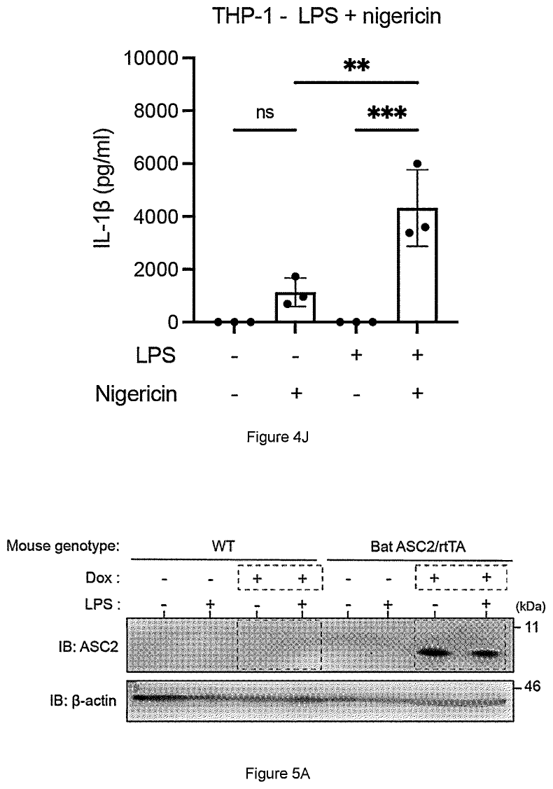

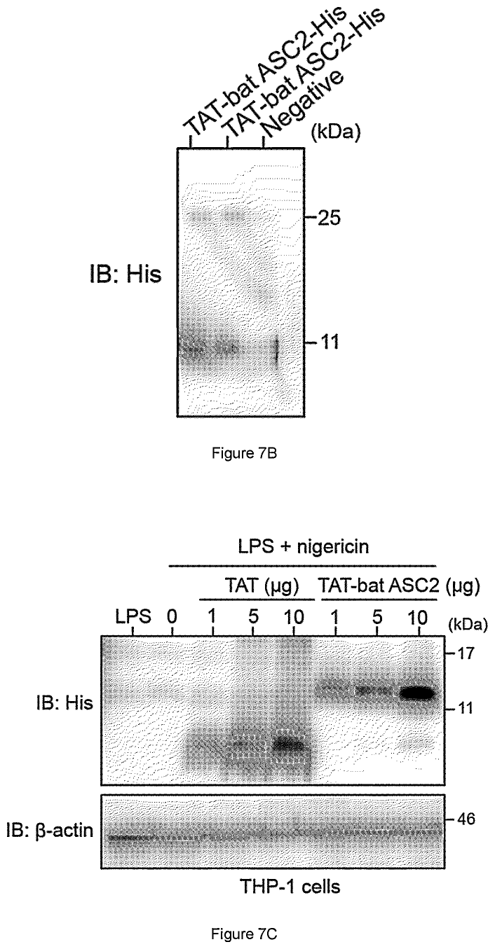

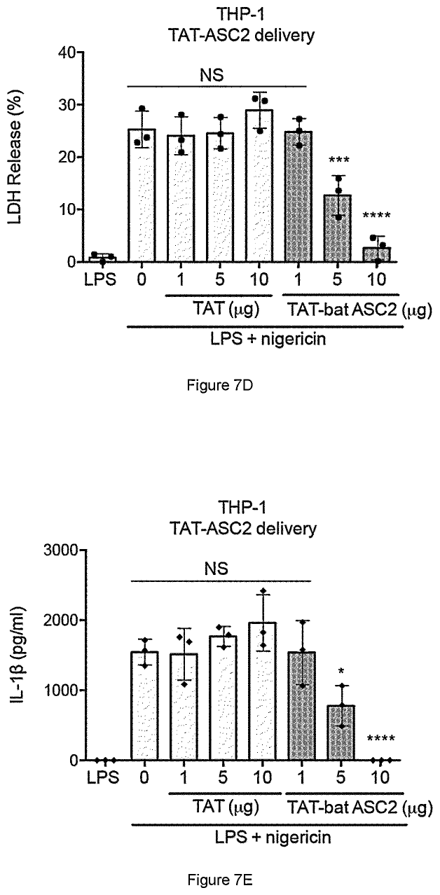

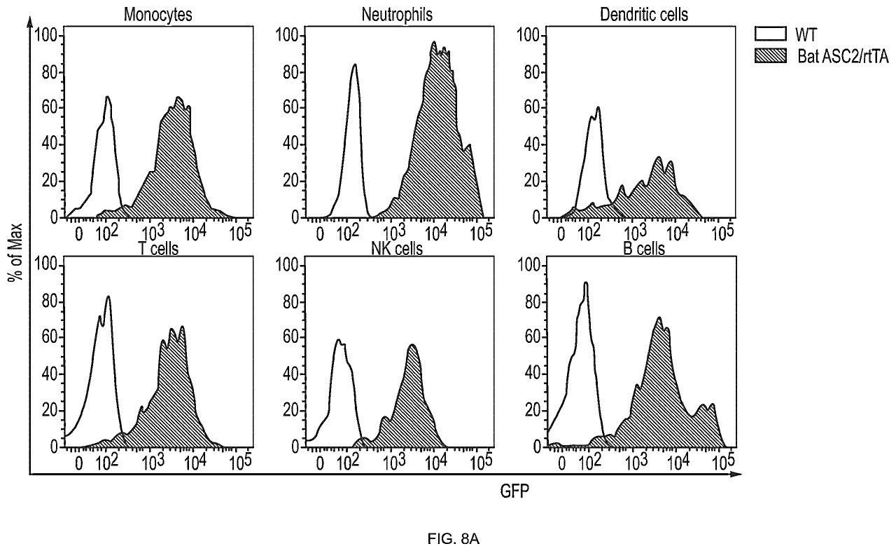

To validate these findings in a more physiological setting of immune cells, ASC2 was overexpressed and its effect in human macrophages was examined. Human monocytic THP-1 cells lack endogenous ASC2 expression⁹ (FIG. 4F and FIG. 4G) and can be differentiated into macrophages with overnight treatment of phorbol 12-myristate 13-acetate (PMA). THP-1 cells stably expressing bat or human ASC2 fused to LSSmOrange were assessed by immunoblots to ensure equal expression (FIG. 4A and FIG. 4H). THP-1 macrophages were primed with lipopolysaccharide (LPS) and then stimulated with a potent NLRP3 activator, nigericin (FIG. 3E, FIG. 3F, FIG. 4B, FIG. 4C, FIG. 4I and FIG. 4J). Consistent with the previous results in HEK293T cells, bat ASC2 inhibited the induction of ASC specks more potently than human ASC2 (FIG. 4B and FIG. 4C). This correlated with significantly more downstream inhibition of pyroptotic cell death determined by the release of lactate dehydrogenase (LDH) (FIG. 3E) and secretion of IL-1β in the supernatants (FIG. 3F). In addition, bat ASC2 also inhibited cell death and IL-1β secretion triggered by another NLRP3 stimulus, monosodium urate crystals (MSU) (FIG. 4D and FIG. 4E), and an AIM2 stimulus, the dsDNA analog poly(dA:dT) (FIG. 3G and FIG. 3H), with a significantly higher potency than human counterpart. To assess if bat ASC2 can be used for a potential treatment strategy targeting human inflammasomes, TAT peptide, a cell-penetrated peptide, was fused to the N-terminus of bat ASC2 to achieve intracellular delivery (FIG. 7A, FIG. 7B and FIG. 7C). TAT peptide is a short cationic peptide derived from HIV-1 TAT protein with wide applications in preclinical models and clinical trials^{39,40}. Treatment of TAT-bat ASC2 fusion proteins led to a dose-dependent inhibition of human NLRP3 inflammasome activation in LPS-primed THP-1 macrophages stimulated with nigericin (FIG. 7D and FIG. 7E). Lack of inhibition by TAT peptide alone indicated the bat ASC2-dependent effect for this inhibition (FIG. 7D and FIG. 7E). Collectively, these data show that bat ASC2 suppresses human inflammasomes by interacting with ASC with a higher potency than human ASC2.

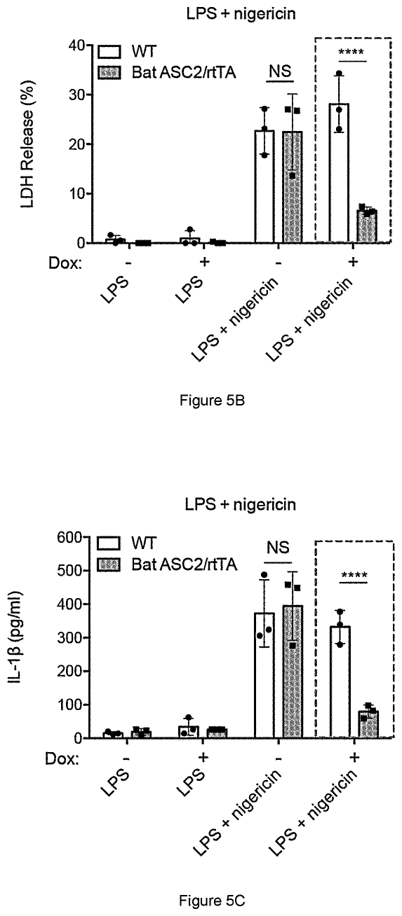

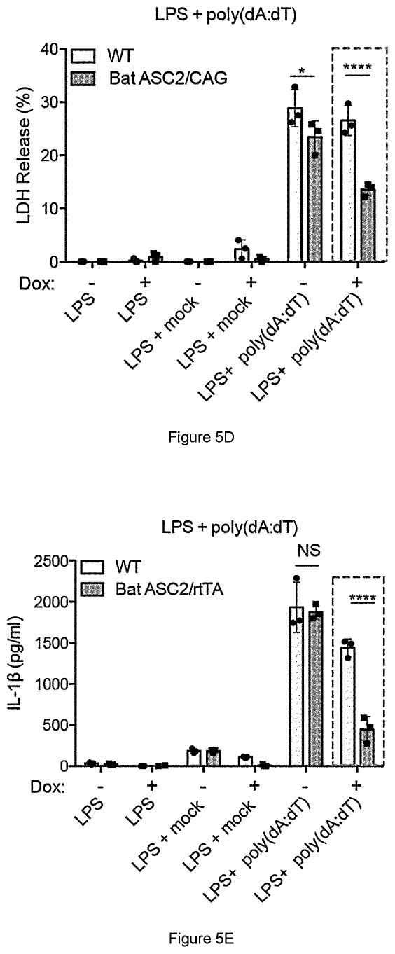

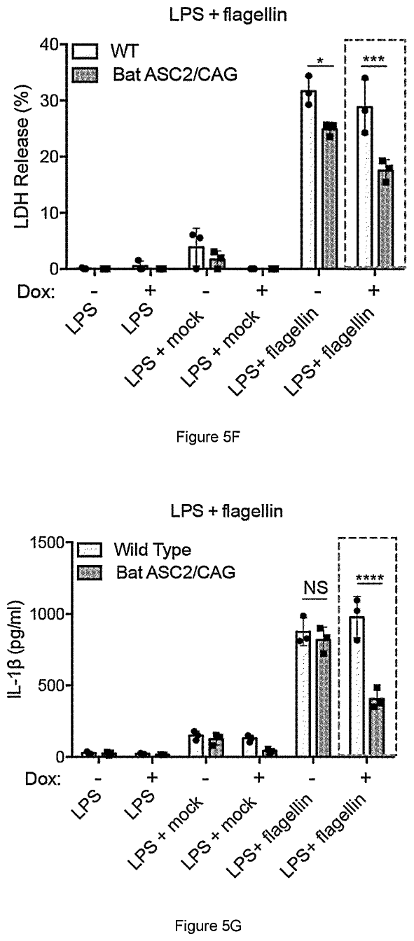

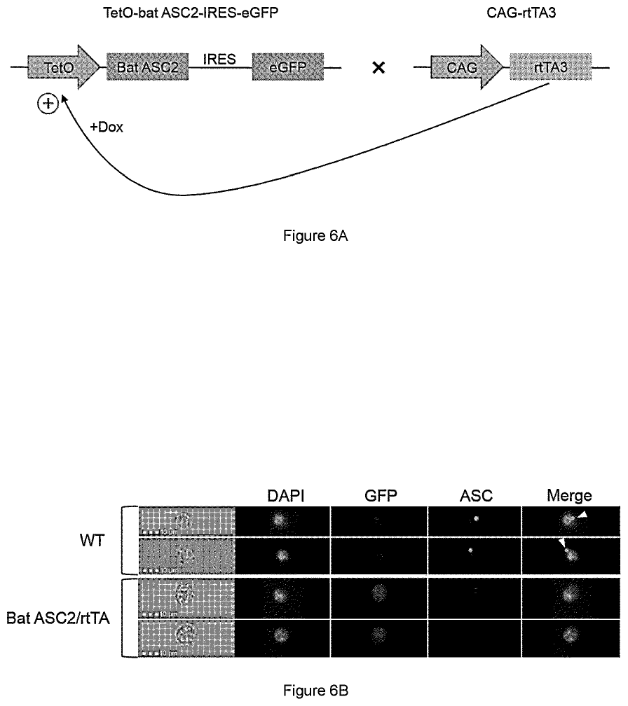

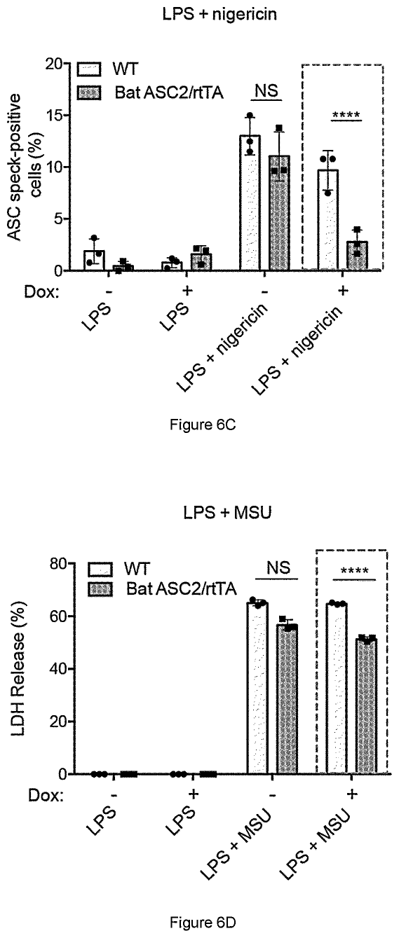

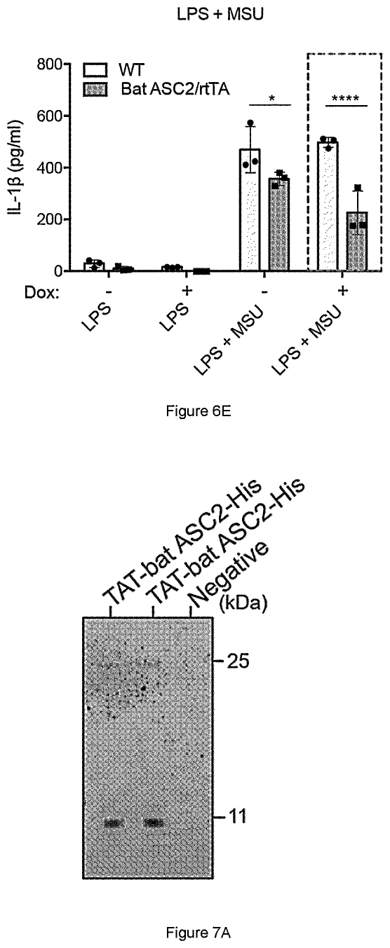

The first transgenic mice containing a bat gene was established to examine the role of bat ASC2 in mouse macrophages and in mouse models. Mice have a ‘clean’ genetic background for ASC2 due to absence of ASC2 locus (FIG. 13). The Tet-on expression system was used to allow the inducible expression of bat ASC2. Transgenic mice with TRE/tetO promoter-directed bat ASC2 and IRES-linked GFP was generated first (FIG. 6A). These transgenic mice were subsequently crossed with CAG-rtTA3 transgenic mice to produce the double positive transgenic mice (bat ASC2/CAG-rtTA3). Progenies were treated with doxycycline (Dox) to induce conditional expression of bat ASC2 in widespread cells/tissues when treated with doxycycline (Dox). The inducible expression of bat ASC2 in bone marrow-derived macrophages (BMDMs) was examined using bone marrows extracted from these mice. A robust induction of bat ASC2 in the presence of Dox and undetectable level in the absence of Dox were demonstrated in BMDMs from the bat ASC2/rtTA mice by immunoblots (FIG. 5A). LPS-primed BMDMs of wild-type (WT) or bat ASC2/rtTA mice were stimulated with nigericin in the presence or absence of Dox. Bat ASC2 expression led to inhibition of NLRP3 inflammasome activation with reduction of ASC speck formation (FIG. 6B and FIG. 6C), pyroptotic cell death (FIG. 5B) and IL-1β secretion (FIG. 5C). The lack of difference in NLRP3 activation for WT BMDMs with or without Dox (FIG. 5B and FIG. 5C) suggested no or minimal effect of Dox on the activation. Similarly, bat ASC2 also suppressed the activation of NLRP3, AIM2 and NLRC4 inflammasomes, triggered by MSU (FIG. 6D and FIG. 6E), poly(dA:dT) transfection (FIG. 5D and FIG. 5E) and flagellin transfection (FIG. 5F and FIG. 5G), respectively. Together, our data demonstrates the ability of bat ASC2 to block multiple ASC-dependent inflammasomes in mouse macrophages.

### Example 3: Delivery of TAT-Bat ASC2 Polypeptide Inhibits the Human Inflammasomes

To assess if bat ASC2 can be used for protein-based treatment strategy targeting human inflammasomes, TAT peptide, a cell-penetrating peptide, was fused to the N-terminus of bat ASC2 to achieve intracellular delivery (FIG. 7A, FIG. 7B and FIG. 7C). TAT peptide is a short cationic peptide derived from HIV-1 TAT protein with wide applications in preclinical models and clinical trials^{39,40} Treatment with TAT-bat ASC2 fusion proteins led to a dose-dependent inhibition of human NLRP3 inflammasome activation in LPS-primed THP-1 macrophages stimulated with nigericin (FIG. 7D and FIG. 7E). Lack of inhibition by TAT peptide alone indicated the bat ASC2-dependent effect for this inhibition (FIG. 7D and FIG. 7E). Therefore, ASC2 polypeptide fused to a cell-penetrating peptide is delivered to immune cells to inhibit inflammasomes as a protein-based treatment approach.

### Example 4: Bat ASC2 is Useful as a Treatment for Inflammatory Diseases, Including Gout

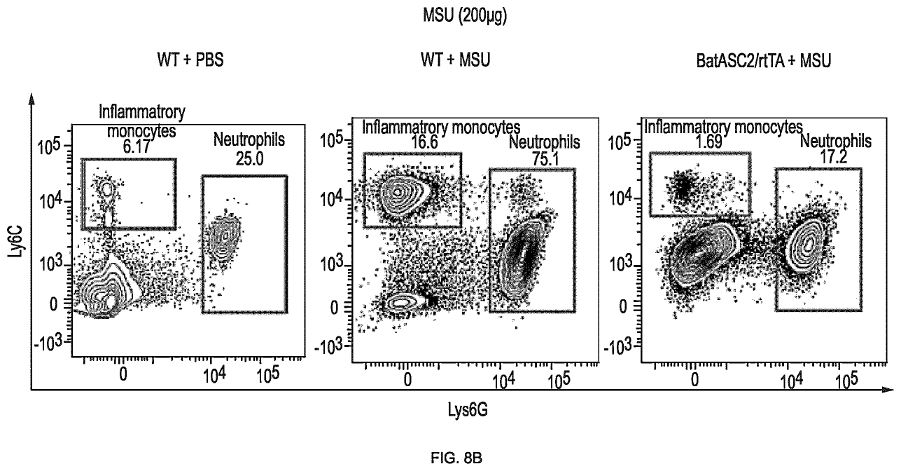

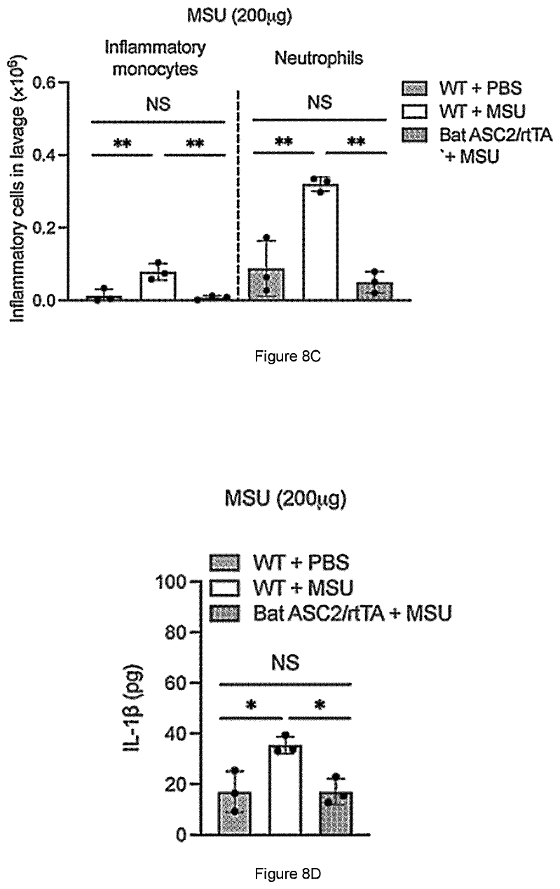

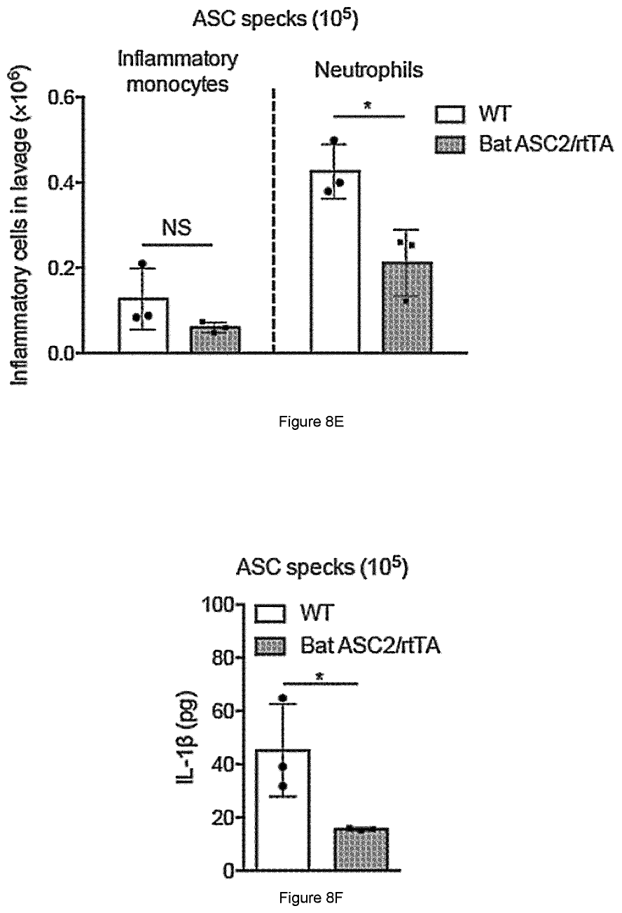

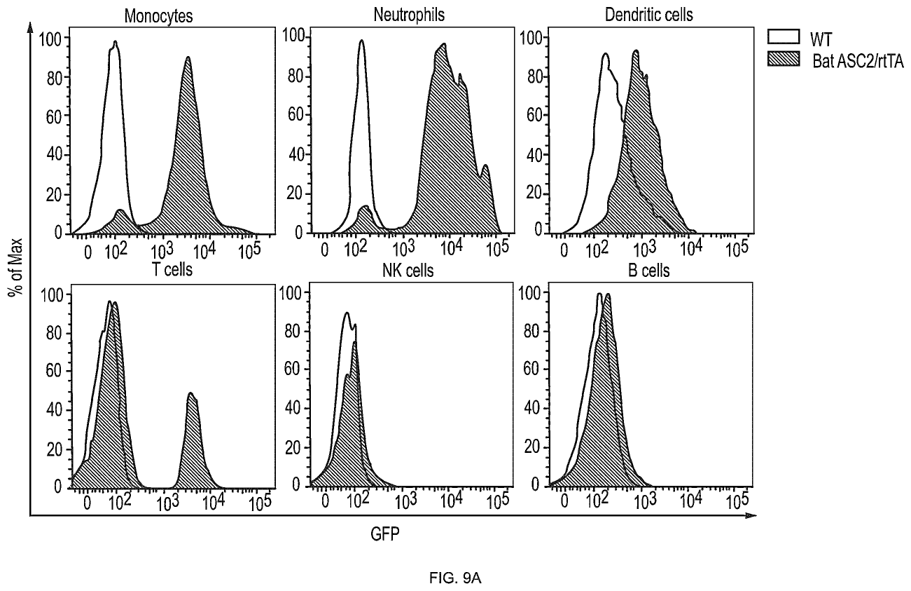

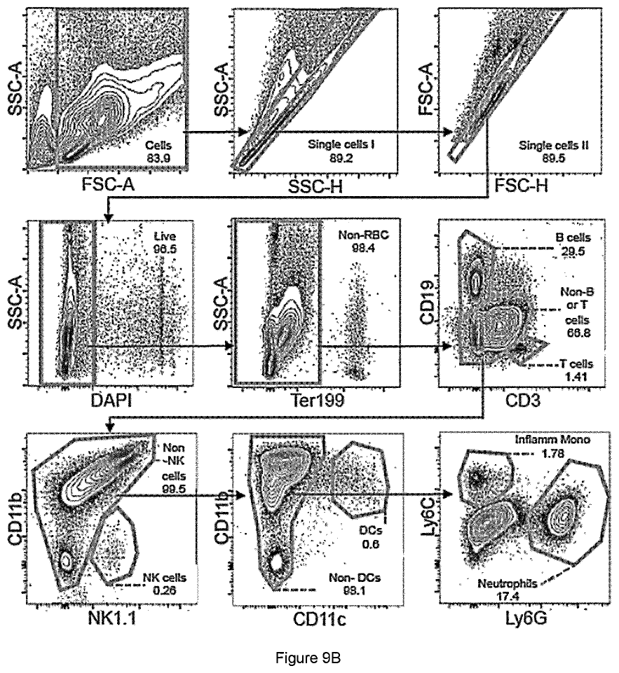

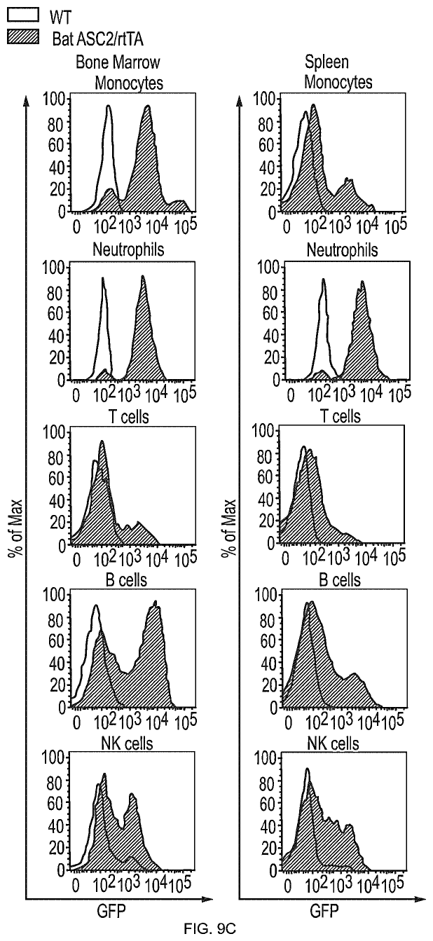

The induction of bat ASC2 expression in vivo was examined. Upon oral administration of Dox for 7 days, widespread bat ASC2 expression in multiple immune cell types including monocytes, as indicated by expression of IRES-linked GFP, was detected in peripheral blood (FIG. 8A), peritoneal lavage (FIG. 9A and FIG. 9B), bone marrow and spleen (FIG. 9C and FIG. 9D) from bat ASC2/rtTA mice. To study the role of bat ASC2 in reducing inflammation in vivo, MSU crystals, the causative agent for gout⁴¹, were intraperitoneally (i.p.) injected into WT and bat ASC2/rtTA mice after Dox induction. The injection of MSU crystals into WT mice resulted in a sterile peritonitis characterized by the recruitment of neutrophils and inflammatory monocytes (FIG. 8B). The recruitment of these inflammatory cells was significantly reduced in bat ASC2/rtTA mice (FIG. 8B and FIG. 8C), correlating with decreased IL-1β release in the peritoneal lavage (FIG. 8D). In addition to their intracellular function, the ASC specks also have extracellular and ‘prionoid’ activities that amplify inflammation in an NLRP3-independent manner in inflammatory diseases^{16,17}. Importantly, the extracellular ASC specks have been implicated in various inflammatory diseases including autoimmune/autoinflammatory diseases, COPD and Alzheimer's diseases. To assess the ability of bat ASC2 to block this cell-derived danger signal, FACS-purified ASC-mPlum specks were i.p. injected to induce peritonitis. The recruitment of neutrophils and IL-1β release were suppressed in bat ASC2/rtTA mice compared to WT mice (FIG. 8E and FIG. 8F). This supports that bat ASC2 inhibits inflammasome activation at the level of ASC in vivo.

### Example 5: Bat ASC2 is Useful as a Treatment for Lethal Viral Infection

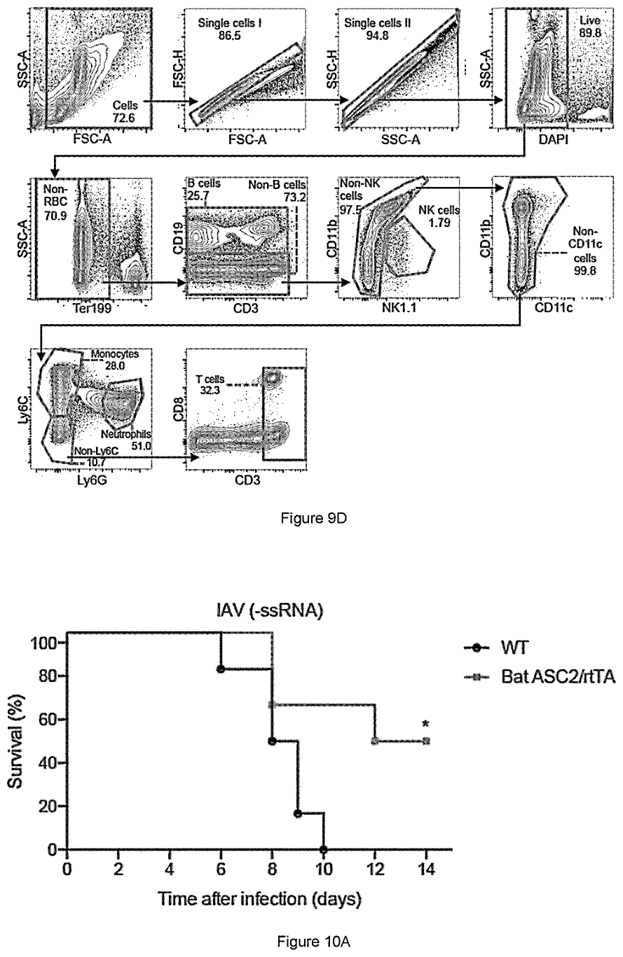

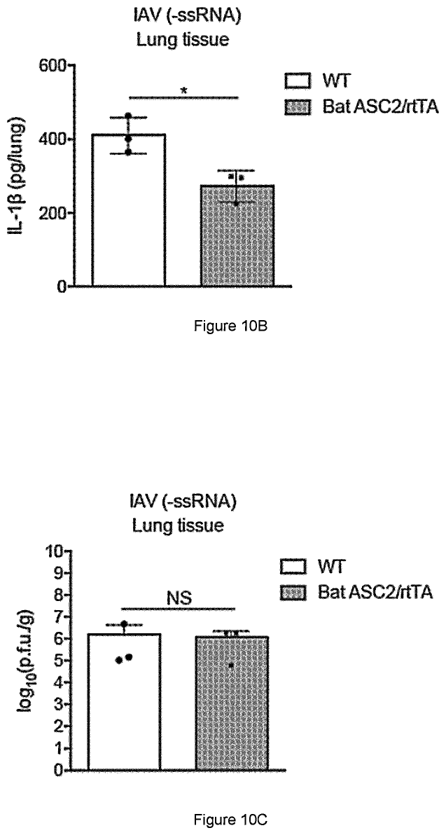

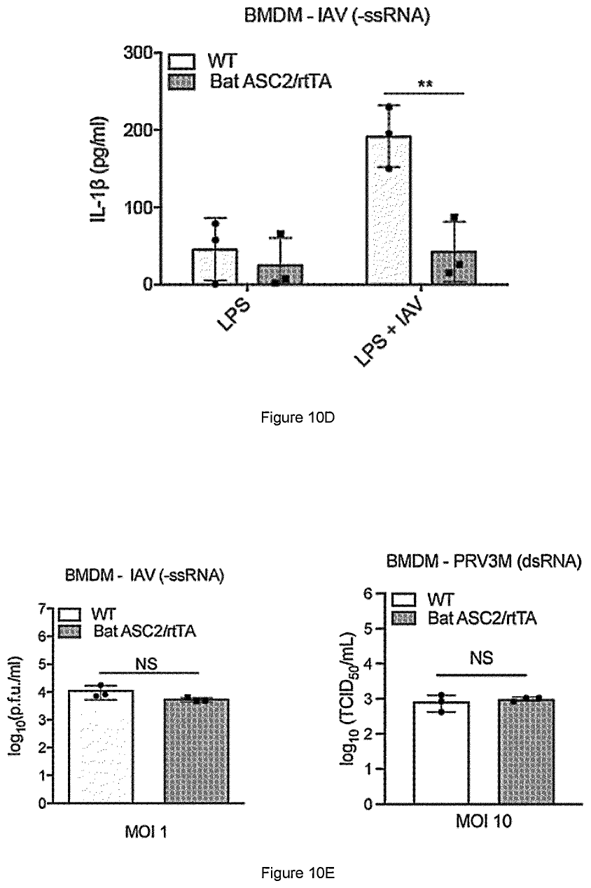

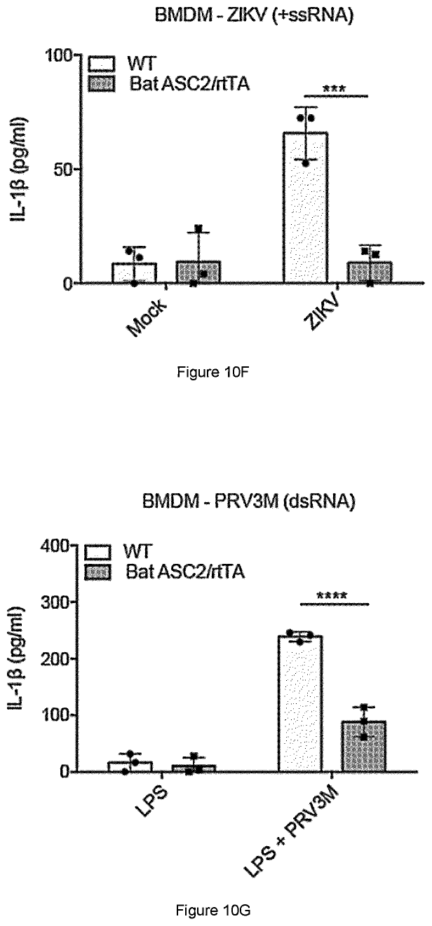

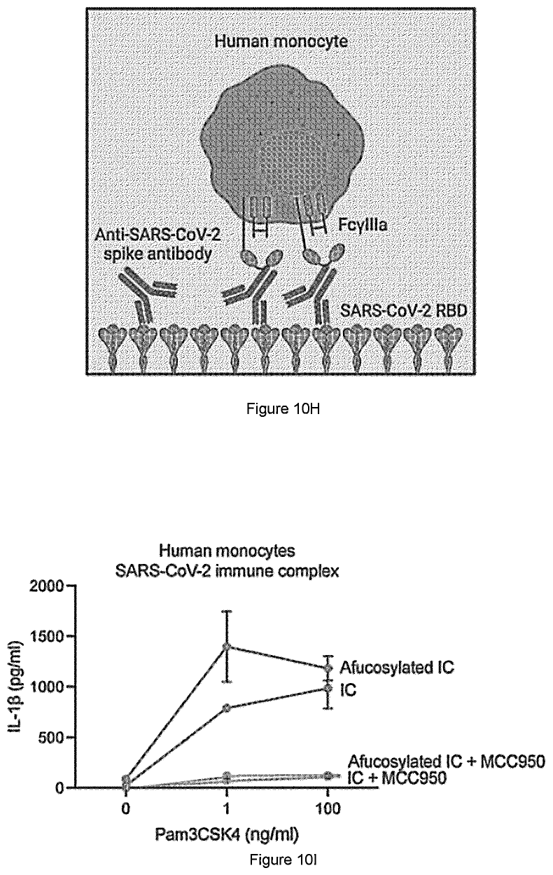

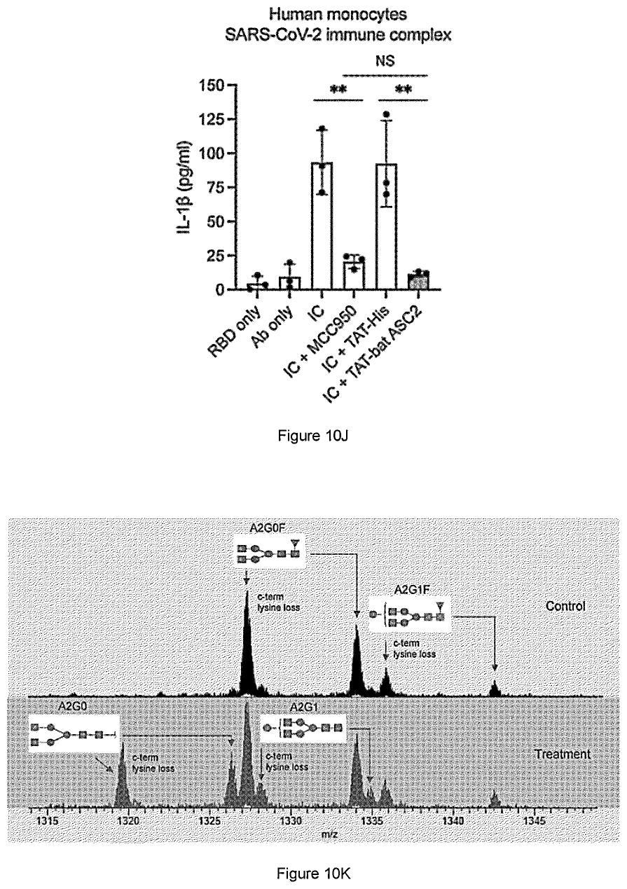

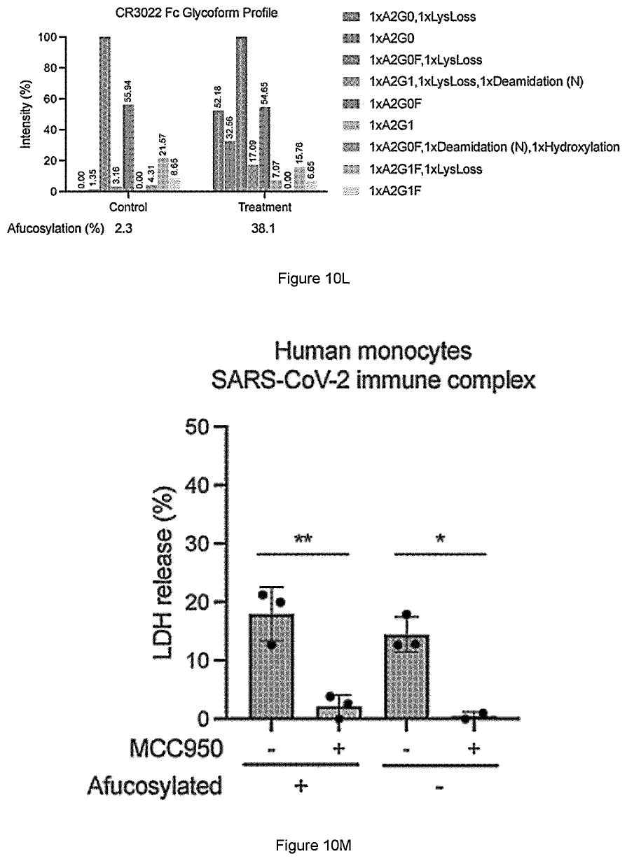

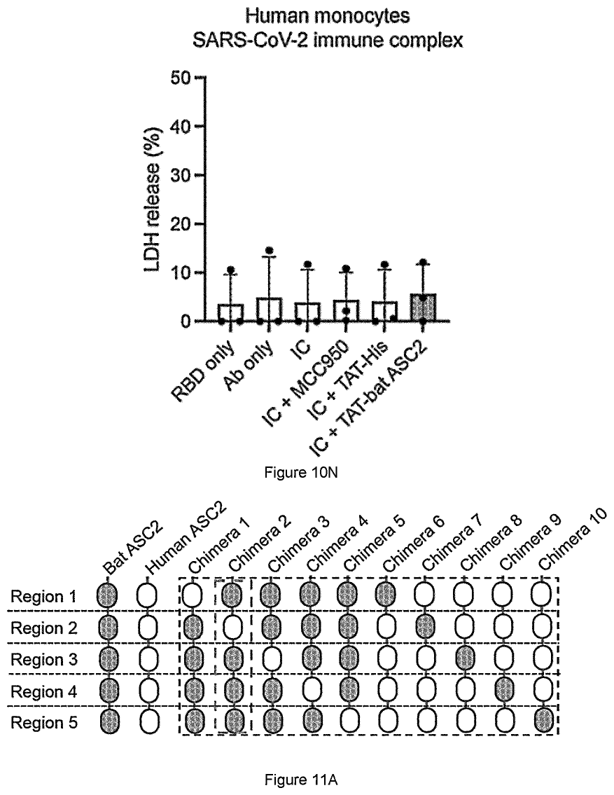

As bats are a special reservoir for emerging viruses, the role of bat ASC2 in lethal viral infection was examined. Influenza A virus (IAV), a negative-sense single-stranded RNA (-ssRNA) virus, is known to activate NLRP3 inflammasome^{20,42,43} and more recently AIM2 inflammasome³⁵. One day after Dox administration, WT or bat ASC2/rtTA mice were infected with H1N1 IAV, strain A/NWS/33, by the intranasal (i.n.) route. At 14 days post-infection (dpi), 100% of WT mice succumbed to disease in contrast to only 50% mortality for the bat ASC2/rtTA mice (FIG. 10A). To assess the extent of inflammation and infection, infected mice were euthanized 3 dpi and lungs were harvested for measurements of cytokines and viral titres. The level of IL-1β was significantly reduced in lung tissue extracts from the infected bat ASC2/rtTA mice (FIG. 10B). In contrast, there was no significant different in the lung viral titres (FIG. 10C). Consistent with the results from in vivo infection, the in vitro IL-1β response to IAV infection was also significantly lower in bat ASC2/rtTA BMDMs compared to WT BMDMs (FIG. 10D), with no obvious difference in viral titres (FIG. 10E). To validate that this finding is not IAV-specific, investigation was expanded to Zika virus (ZIKV)⁴⁴, a positive-sense (+) ssRNA virus, and Pteropine orthoreovirus 3 (PRV3M)^{38,45}, a bat-borne zoonotic reovirus of double-stranded RNA (dsRNA). Similarly, the IL-1β secretion triggered by ZIKV (FIG. 10F) and PRV3M (FIG. 10G) was substantially inhibited in bat ASC2/rtTA BMDMs, without affecting the viral load for PRV3M (FIG. 10E). These data demonstrate that bat ASC2 dampens virus-induced inflammation in response to three different types of RNA virus. Altogether, expression of bat ASC2 reduces both stress-related and virus-induced inflammation and ameliorates inflammatory diseases.

To confirm this observation for SARS-COV-2, the inflammasome activation in the context of immune complex-induced inflammation was examined. Several studies have recently shown that high afucosylation of anti-SARS-COV-2 IgG correlates with COVID-19 severity and promotes inflammation by immune complex-mediated activation of FcγRs and release of cytokines including IL-β^{82,83,84}. However, the mechanism of the immune complex-induced IL-1β is still unknown. To determine its mechanism, human monocytes were treated with immune complexes formed by the receptor-binding domain (RBD) of SARS-COV-2 spike protein and anti-RBD monoclonal antibody (FIG. 10H). To validate the role of afucosylation, the anti-RBD monoclonal antibody with 38% afucosylation, comparable to the level of high afucosylation seen in severe patients, was generated by enzymatic cleavage of the fucose (FIG. 10K and FIG. 10L). As TLR2 has recently been shown to sense SARS-COV-2 resulting in the production of inflammatory cytokines⁸⁵, various concentrations of the TLR2 ligand, Pam3CSK3, was used to study the role of priming. The data revealed that both afucosylation and priming enhanced the immune complexed-induced IL-1β (FIG. 10I). Importantly, both IL-1β secretion and cell death were abolished by inhibition of NLRP3 with a small-molecule inhibitor, MCC950, indicating NLRP3-dependent activation of inflammasome by immune complexes (FIG. 10I and FIG. 10M). To examine if bat ASC2 can block this, primed human monocytes activated with the SARS-COV-2 immune complexes were treated with TAT-bat ASC2 (FIG. 10J). Delivery of TAT-bat ASC2 significantly reduced the IL-1β secretion triggered by SARS-COV-2 immune complex (FIG. 10J) but not cell death (FIG. 10N), likely due to the short time frame of experiment not yet inducing cell death. These data demonstrate that bat ASC2 also dampens virus-induced inflammation in response to SARS-COV-2 immune complex-induced inflammation.

### Example 6: The Polypeptide of a Mutant Human ASC2 Exhibits Enhanced Potency for Inhibiting the Inflammasomes

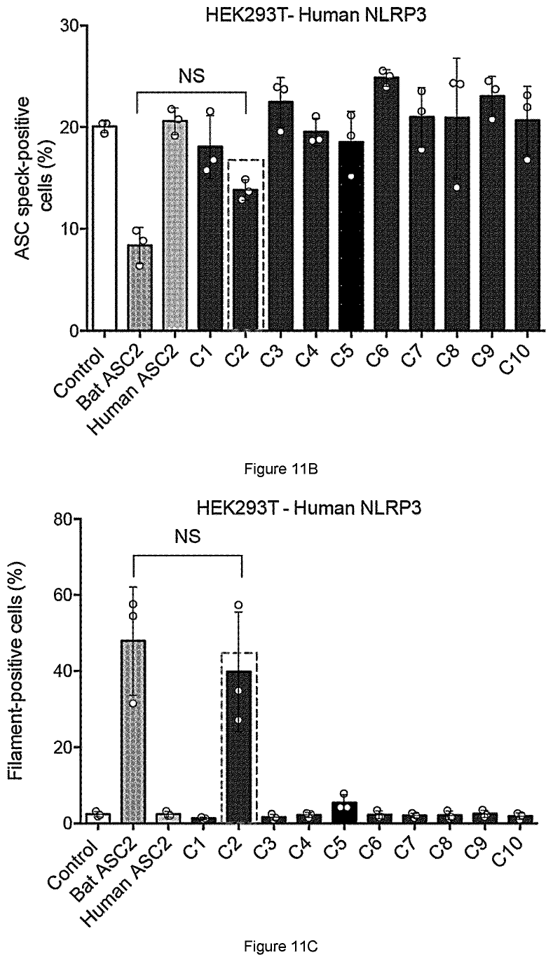

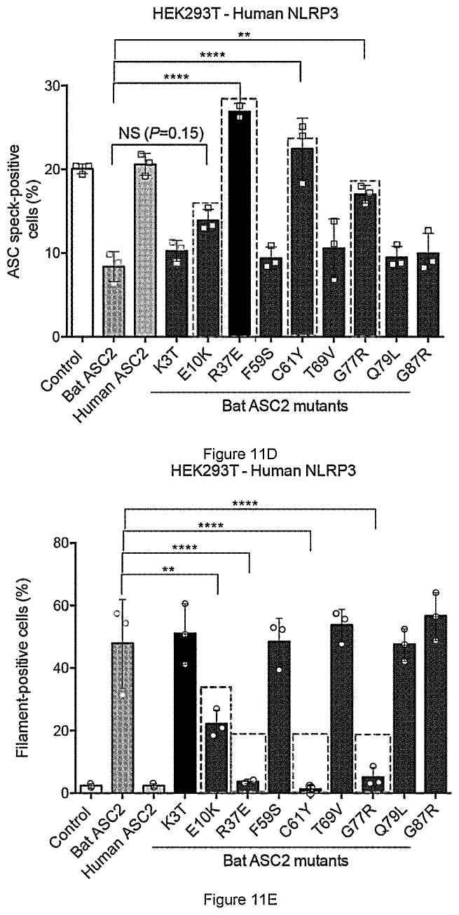

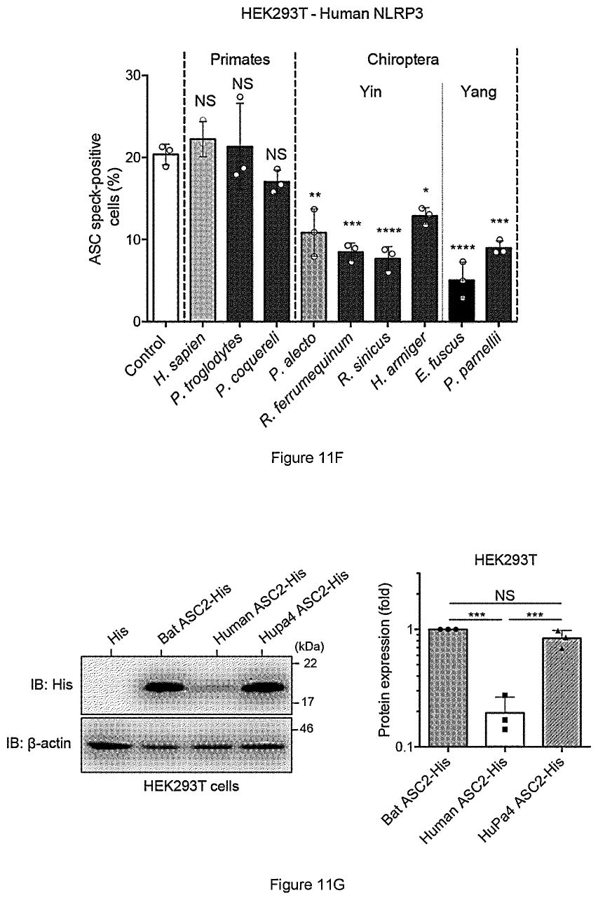

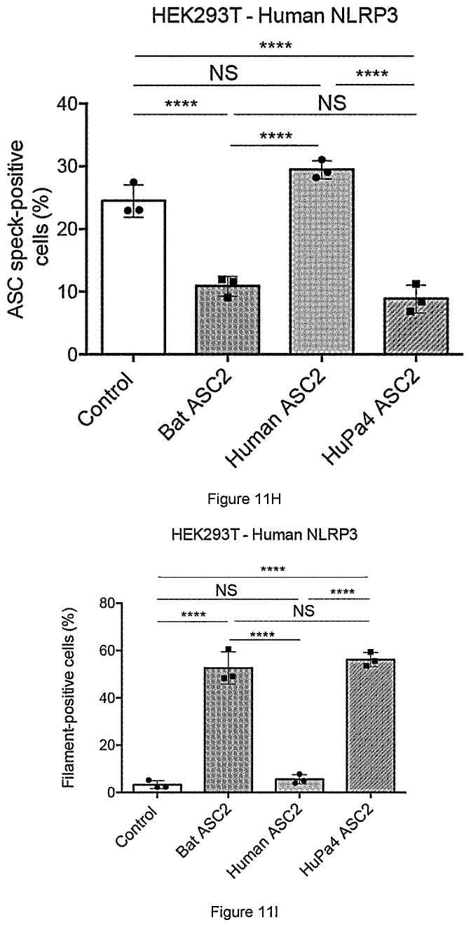

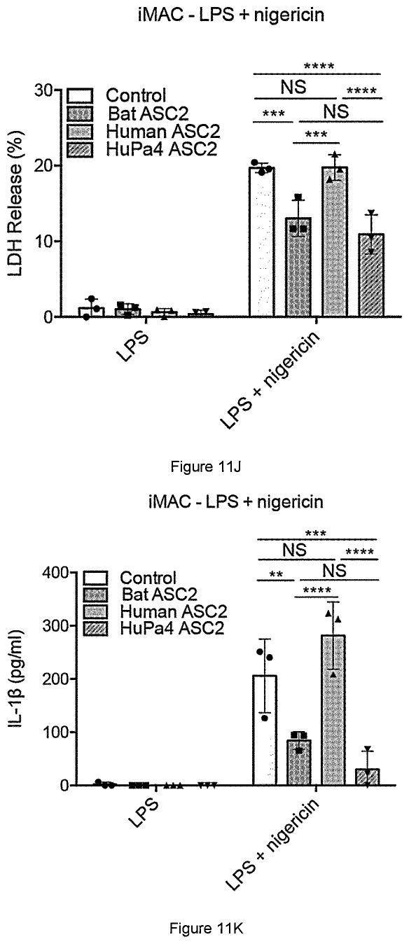

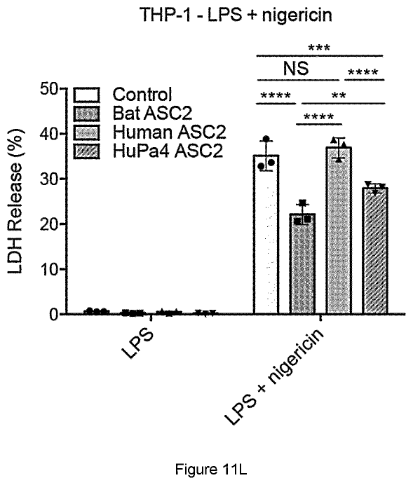

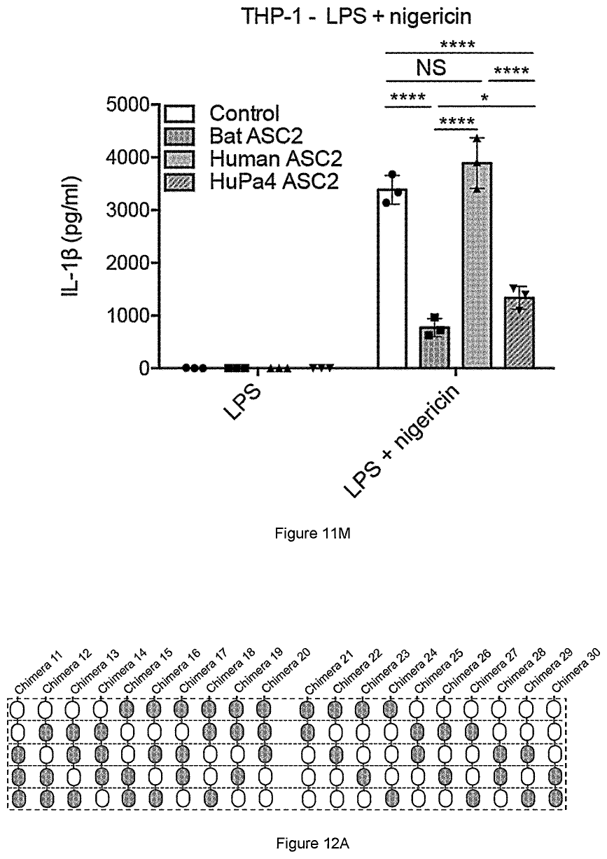

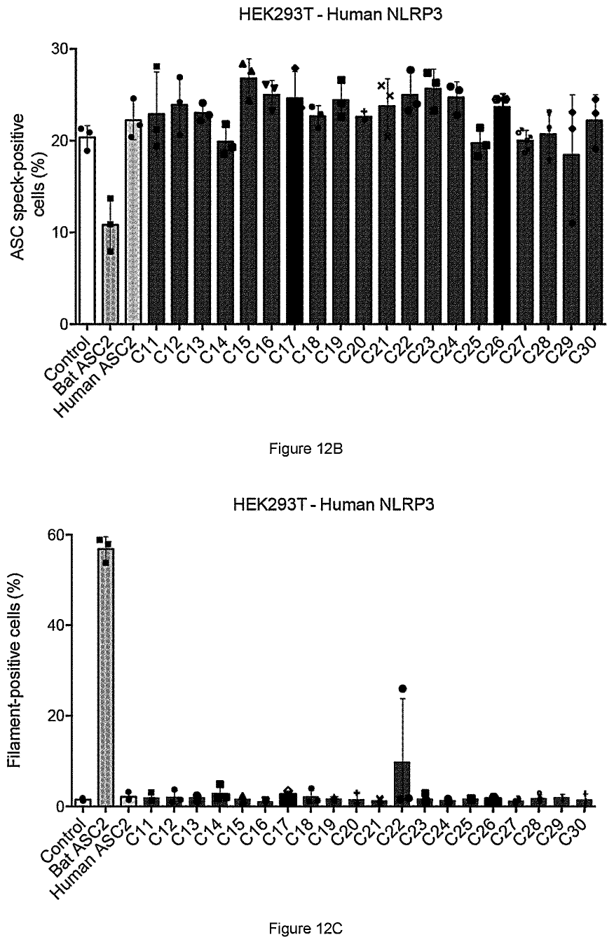

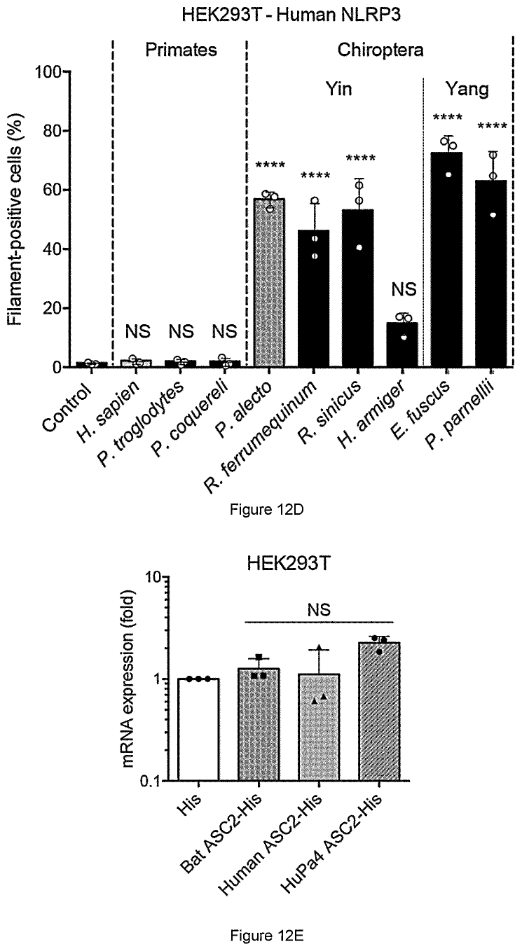

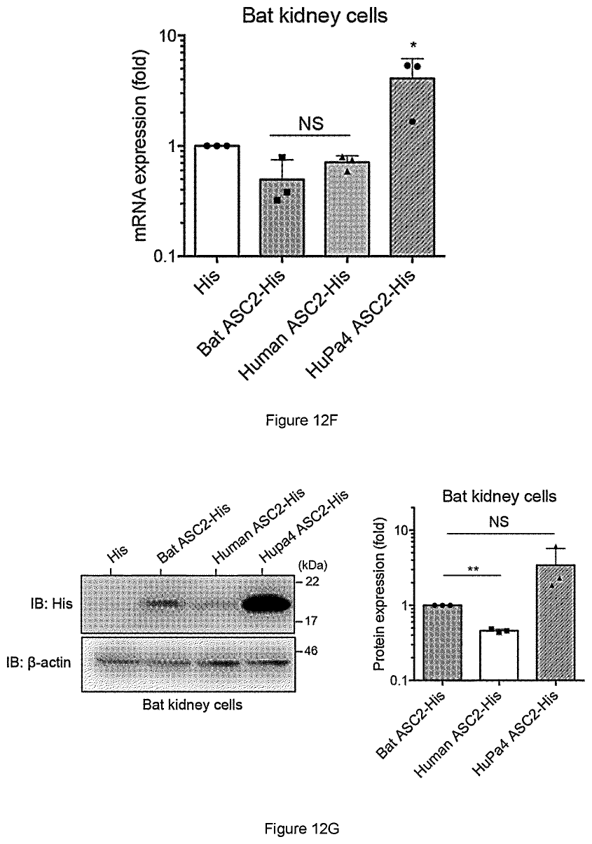

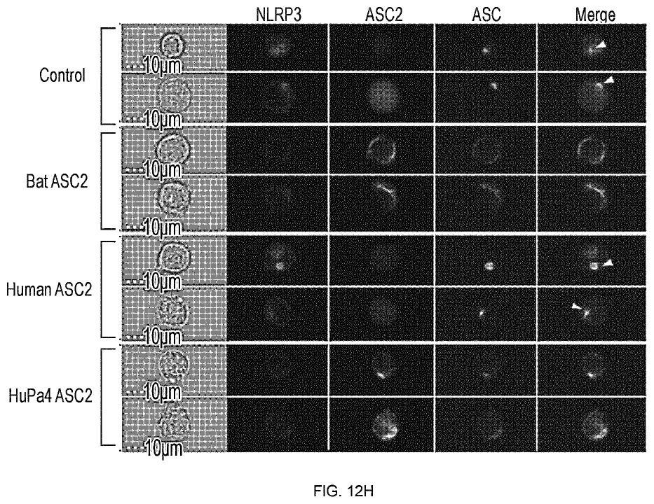

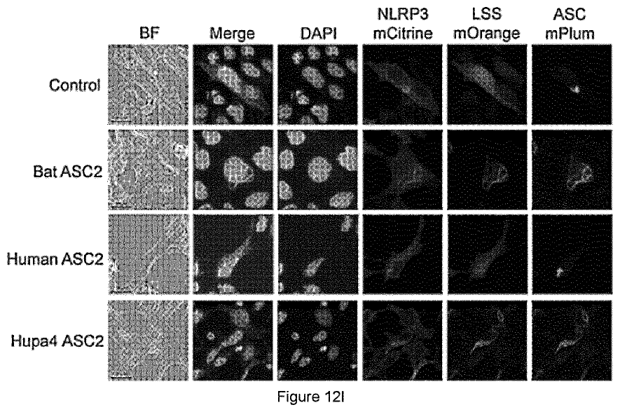

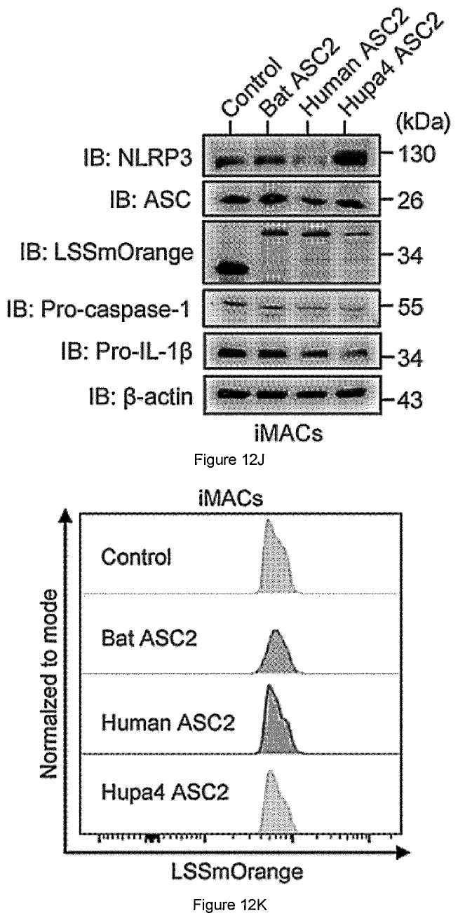

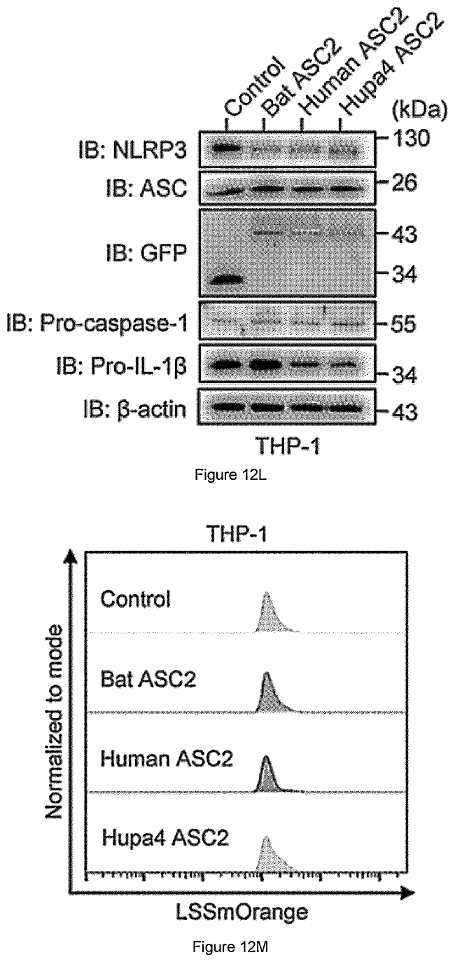

To map the regions responsible for the gain-of-function of bat ASC2, a chimera between bat and human ASC2 was generated by swapping the five different regions (FIG. 11A and FIG. 12A). These five regions were divided based on the individual α-helical structures of ASC2^{46,47} after combining the smaller 3^{rd} and 4^{th} α-helix. Hence, 30 different chimeras in total were generated (FIG. 11A and FIG. 12A). Interestingly, only ‘chimera 2’ among all the chimeras exhibited comparable inhibition of ASC speck activation and similar filament formation to wild-type bat ASC2 (FIG. 11B, FIG. 11C, FIG. 12B and FIG. 12C). This suggests that all four regions (number 1, 3, 4 and 5) are required for the gain-of-function of bat ASC2. Alignment of bat and human ASC2 amino acid sequences (FIG. 1B) showed 71% identity with 26 mismatches.

To further identify the key residues, site-directed mutagenesis was performed for bat ASC2 by substituting the mismatch residues between bat and human ASC2 within these 4 regions. Functional analysis revealed four mutations of bat ASC2 (E10K, R37E, C61Y and G77R) that disrupted the function of inflammasome inhibition and formation of filaments (FIG. 11D and FIG. 11E). These four residues include one interface residue (C61) and three non-interface residues (E10, R37 and G77), predicted based on interfaces of PYD domain of human ASC⁴⁸. However, functions of these four residues in PYD domain have not been reported so far. Chiroptera is a diverse mammalian order that consists of more than a thousand bat species. To assess if our findings for ASC2 of P. alecto from the Yinpterochiroptera suborder are also true across multiple bat species, we extended our study to three other bats from the Yinpterochiroptera and two bats from the other suborder, Yangochiroptera (FIG. 14). The ability to inhibit human NLRP3 inflammasome was conserved in ASC2 of all the 6 bat species, in contrast to ASC2 of three primates examined (FIG. 11F and FIG. 12D). Importantly, the 4 key residues identified in ASC of P. alecto were conserved in ASC2 of these 6 bat species, except one residue (C61) was not identical for some bats (C61R). To confirm the role of these 4 key residues, site-directed mutagenesis for human ASC2 was performed to substitute these 4 sites simultaneously with the ones in bat ASC2, which was named ‘hupa4’ ASC2. Hupa4 ASC2 exhibited enhanced protein expression in both HEK293T cells (FIG. 11G and FIG. 12E) and PaKi cells (FIG. 12F and FIG. 12G). Importantly, it also gained the ability to inhibit NLRP3 inflammasome and form filaments in the reconstituted HEK293T cells, resembling the wild-type bat ASC2 (FIG. 11H, FIG. 11I, FIG. 12H and FIG. 12I). This gain-of-function of hupa4 ASC2 was further validated in the mouse immortalized macrophages (IMACs) (FIG. 11J and FIG. 11K) and human THP-1 macrophages (FIG. 11L and FIG. 11M) stably expressing equal ASC2 (FIG. 12J, FIG. 12K, FIG. 12L and FIG. 12M). Overall, 4 key residues identified are responsible for enhanced protein expression and gain-of-function of bat ASC2. The mutant human ASC2 (hupa4 ASC2) with changes of the 4 key residues in bat ASC2 exhibits increased expression and gain-of-function. An advantage of using such human mutant protein over the wild type bat protein for therapies is minimization of potential immunogenicity and side effects associated with a foreign antigen/protein.

### Example 7: High-Throughput Screening of ASC Inhibitors

### 7.1 Materials and Methods

Plasmids. Vectors were constructed by standard molecular cloning with pcDNA3.1 (+) as the backbone. Restriction enzyme sites used include NheI, AgeI (introduced), HindIII, BamHI, XhoI, and XbaI from 5′ to 3′ sequentially. Human ASC PYRIN domain (PYD) was PCR amplified, and the E13R, K21E/K22E, and F59E mutations were introduced by overlapping PCR. The NanoBiT tag LgBiT was amplified by PCR from pcDNA3.1.Gα13-LgB106 (Addgene #134364), and SmBIT was oligo annealed. The N-terminal and C-terminal inserts of human ASC PYD or NanoBiT tags were flanked by AgeI/HindIII and BamHI/XhoI, respectively. The linker (GSSGGGGSGGGGSSG) flanked by HindIII/BamHI and C-terminal His₁₀-tag flanked by XhoI/XbaI was oligo annealed. The gene fragments were introduced into the backbone by single, double, or triple insertions. The resulting vectors were sequenced using CMV-F and BGH-rev primers.

Cell Culture and Transfection. HEK293T cells were cultured in modified Eagle's medium (DMEM) with 10% fetal bovine serum in humidified 37° C. incubator with 5% CO₂. Cells were seeded at a density of 3×10⁵ cells/mL one day prior to transient transfection using Fugene 6 transfection reagent (Promega) according to the manufacturer's instructions. At 24 h post-transfection, cells are harvested for analyses.

NanoBiT and Inhibition Assay. White opaque plates were coated with 50 μL of 0.001% poly-L-lysine for at least 15 min at 37° C. and air-dried before seeding 3×10⁵ cells/mL HEK239T cells. The following day, cells were transiently transfected using Fugene 6 transfection reagent (Promega) according to the manufacturer's instructions. At 24 h post-transfection, cells were washed once with 100 μL Opti-MEM followed by changing media with 100 μL Opti-MEM, and 25 μL Nano-Glo Live Cell Reagent (Promega) is added according to the manufacturer's instructions. Luminescence is immediately quantified using BioTek Cytation 5 with a gain of 135 and an integration time of 1 s.

### 7.2 Results and Discussion

Reporter systems may be used to perform target-based high-throughput screening (HTS) of ASC inhibitors. These such systems are bimolecular fluorescence complementation (BiFC) such as split Venus⁶⁹⁻⁷¹, bimolecular luciferase complementation (BILC) such as NanoLuc Binary Technology (NanoBiT)⁷², and NanoBRET^{73,74}. These systems allow detection of ASC-ASC protein-protein interaction, which is required for ASC speck formation and inflammasome activation^{75,76}. To monitor this interaction in HTS assays, a full-length ASC or one of its domains, PYD or CARD domain, is used in each of the reporter systems. In addition, PYD and CARD domain each have three interfaces critical for its homotypic domain interaction⁷⁷⁻⁸⁰. Therefore, the interaction of one, two, and three interfaces can be monitored and targeted in HTS, using a PYD or CARD domain containing mutations of key residues in two, one, and zero interfaces, respectively. To establish the background signal for each assay, a domain or full-length protein containing mutations of key residues in each of three interfaces of PYD and/or CARD domain of ASC is generated. All assays are being developed and performed in an epithelial cell line such as HEK293T cells or an immune cell line such as THP-1 cells. Performing the HTS in these cell lines allows identification of inhibitors with high potency in an intracellular environment and good cell membrane permeability. In HTS of compound libraries, a candidate of ASC inhibition disrupting the ASC-ASC interaction is expected to reduce the reporter signals significantly. For proof-of-concept, bat ASC2, a potent inhibitor of ASC, is introduced into the cells by an expression vector or intracellular protein delivery and used as an inhibitor in the assays. Alternatively, an ASC-inhibiting peptide⁸¹ is used. The domains or full-length proteins fused to the BiFC, NanoBIT, or NanoBRET assay pairs are expressed transiently or stably in the cells. The HTS assays for ASC inhibitor can be also extended to other key inflammasome protein-protein interactions. Examples include, but are not limited to, NLRP3-NLRP3 (full-length NLRP3-full-length NLRP3, NLRP3 PYD-NLRP3 PYD and NLRP3 NBD-NLRP3 LRR), NLRP3-ASC (full-length NLRP3-full-length ASC and NLRP3 PYD-ASC PYD), ASC-caspase-1 (full-length ASC-full-length caspase-1 and ASC CARD-caspase-1 CARD) and caspase-1-caspase-1 (full-length caspase-1-full-length caspase-1 and caspase-1 p10-caspase-1 p10).

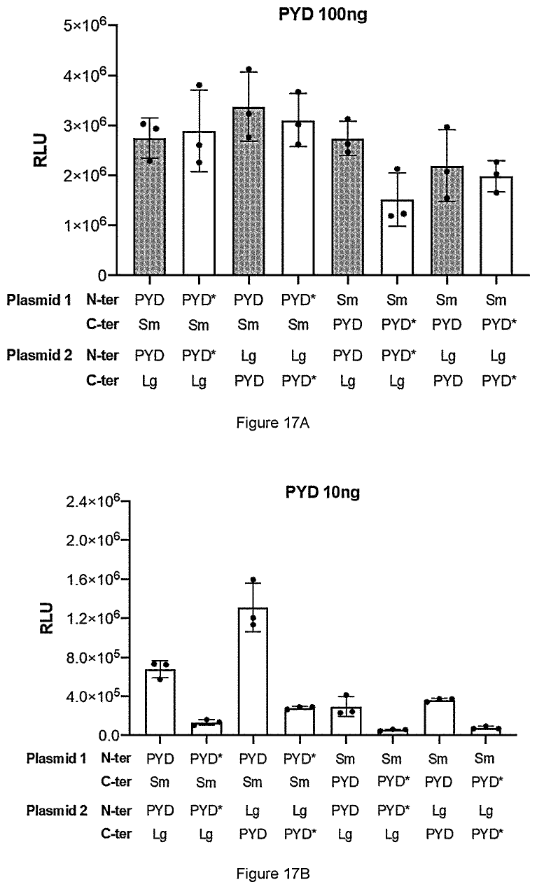

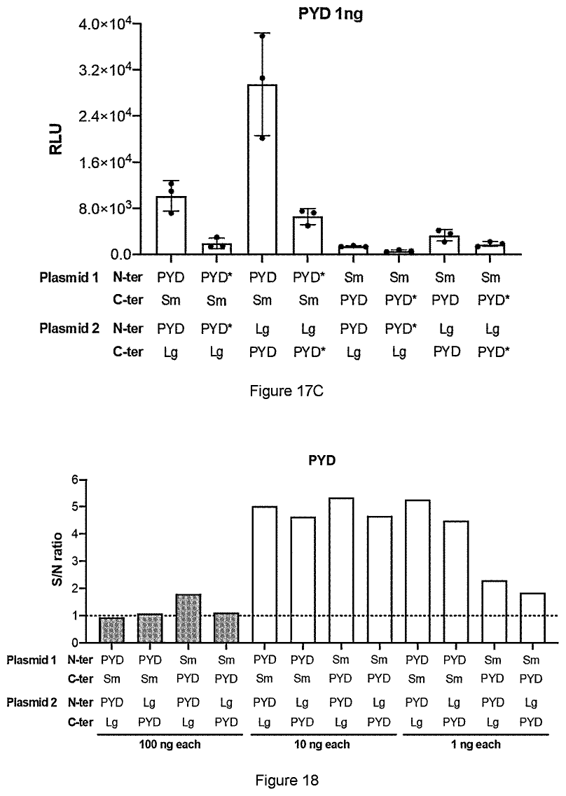

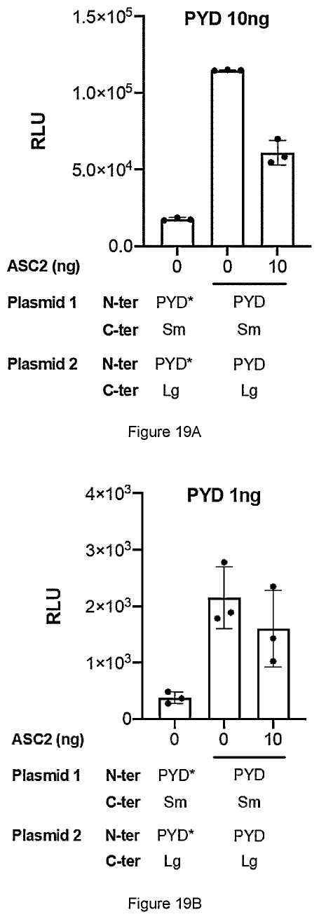

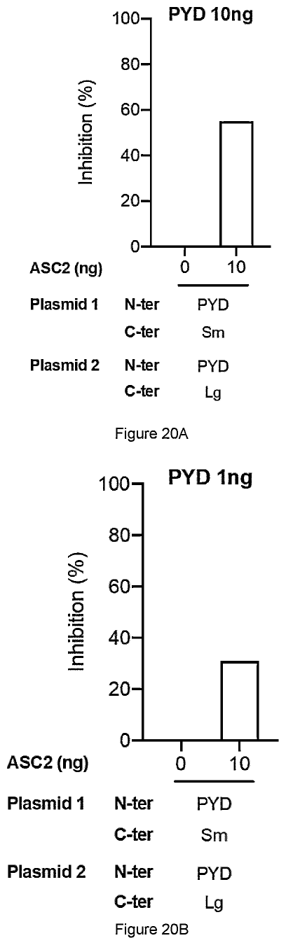

The NanoBIT system was used to monitor the PYD-PYD interaction of human ASC. The human ASC PYD domain was fused with either of the two NanoLuc components, Large BIT (LgBIT; SEQ ID NO:20) and Small BIT (SmBIT; SEQ ID NO:19). When the PYD domains interact, LgBIT and SmBIT can complement and release a luminescence signal upon addition of the substrate, furimazine (FIG. 16). To first determine the best pair for ASC-ASC protein interaction, two plasmids (encoding PYD fused at the N- or C-terminus with LgBiT or SmBIT) were co-transfected in HEK293T cells at different doses, and the signal-to-noise ratio (S/N ratio) was calculated by dividing the wild-type PYD signal by the mutant PYD signal. As the E13R, K21E/K22E, and F59E mutations in PYD have been shown to disrupt each of three key interfaces of PYD-PYD interaction and formation of filament⁷⁹, PYD with these three mutations was used to establish the background LgBiT-SmBIT interaction signal. A transfection dose of 10 or 1 ng per plasmid resulted in a better S/N ratio (FIG. 17A, FIG. 17B, FIG. 17C and FIG. 18) and a representative pair with 10 or 1 ng dose was thus used for the inhibition assay to determine the robustness of the ASC inhibitor drug screening assay. For the inhibition assay, two plasmids (PYD fused with LgBiT or SmBIT) along with bat ASC2 were co-transfected in HEK293T. Transfection of bat ASC2 was able to inhibit PYD-PYD protein-protein interaction, thereby preventing the complementation of LgBiT and SmBIT, and resulting in a decrease in the luminescence signal (FIG. 19A, FIG. 19B, FIG. 20A and FIG. 20B).

### Example 8: Ability of TAT-Hupa4 to Inhibit Inflammasome Activation in Multiple Models

### 8.1 Materials and Methods

Reagents. LPS-B5 Ultrapure (tlrl-pb5lps), nigericin (tlrl-nig) were purchased from InvivoGen. α-synuclein preformed fibrils (ASF-1001-01) were purchased from rPeptide. Talabostat (Val-boroPro; PT100) was purchased from MedChemExpress and reconstituted in Dimethyl sulfoxide (DMSO). TAT-hupa4-His recombinant protein was generated and purified from E. Coli by ChinaPeptides. TAT-His control peptide was generated by GeneScript. Anti-His tag monoclonal antibody were purchased from ThermoFisher Scientific. Anti-β-actin monoclonal antibody (A2228) was purchased from Sigma-Aldrich. Anti-mouse horseradish peroxidase (HRP)-conjugated secondary antibodies were purchased from R&D Systems.

Cells. Human peripheral blood mononuclear cells (PBMCs) were extracted from blood cones containing concentrated blood cells from plasma donors to the Health Sciences Authority Singapore's Blood Bank. Informed written consent has been obtained from the donors prior to blood donation. PBMCs were cryopreserved with 90% fetal bovine serum (FBS, Biological Industries) and 10% DMSO, and seeded into 96-well plate in RPMI 1640 medium containing 10% FBS (Biological Industries). Primary human epidermal keratinocytes (adult) (HEKa) were purchased from ATCC® (PCS 200-011™) and cultured in keratinocyte serum-free medium (KSFM) by Gibco supplemented with 25 μg/mL bovine pituitary extract and 0.2 ng/ml human recombinant epidermal growth factor.

TAT-hupa4 delivery. Frozen PBMCs were thawed from −80° C. and recovered in RPMI with 10% FBS (R10) for 2 h. Cells were seeded at 4×10⁶/mL into 96-well plates and incubated for 1 h. Cells were unprimed or primed with 1 μg/mL LPS for 3 h followed by delivery of different concentrations of TAT-His or TAT-hupa4 (1, 4, 8, 16, 24 μM) for 1 h in serum-free RPMI (R0). Subsequently, residual proteins were washed away with fresh R0 and cell media was replaced with R0 containing 6.7 μM nigericin for 1 h or 5 μM pre-formed fibril α-synuclein for 24 h. Cell-free supernatants were collected for LDH release assay and IL-1β detection by ELISA and cells were harvested for immunoblot analysis. HEKa cells were seeded at 2×10⁵/mL into 96-well plates and incubated overnight. The following day, cells were activated by 2 μM talabostat in 0.5% DMSO+KSFM media for 8 h. Subsequently, talabostat was removed and replaced with fresh KSFM media containing different concentrations of TAT-His or TAT-hupa4 (16, 8, 4, 1 μM) for 1 h. The proteins were removed after 1 h and 100 μL fresh KSFM was used to wash away residual proteins. Cells were then continued to be activated by 2 μM talabostat for another 15 h. Cell-free supernatants were collected for LDH release assay and IL-1β detection by ELISA and cells were harvested for immunoblot analysis.

LDH release assay. Cytotoxicity induced by various stimuli was assessed by the release of cytosolic LDH. Cell culture media was collected and centrifuged at 400 g (HEKa) or 500 g (PBMCs) for 5 min at 4° C. and LDH release was quantified in the cell-free supernatants using Cytotoxicity Detection Kit PLUS (Roche). The kit was used according to the manufacturer's instructions. The percentage of LDH release was calculated as LDH release (%)=(Experiment value−Negative control)/(Positive control−Negative control)×100. Negative control is defined as the baseline LDH release from untreated cells, while positive control is the maximum LDH release from cells treated with lysis solution.

ELISA. Human IL-1β in cell-free supernatants were quantified by ELISA according to the manufacturer's instructions (R&D Systems). Filtered 1% bovine serum albumin (BSA) in phosphate-buffered saline (PBS) was used as blocking buffer and assay diluent. 3,3′,5,5′-tetramethylbenzidine (TMB) chromogen solution (Invitrogen) and TMB stop solution (VWR) were used in the assay. Absorbances at 450 nm and 570 nm were read using Cytation 5 imaging reader (BioTek).

Immunoblot analysis. After removal of media, cells were lysed in lysis buffer containing complete ULTRA protease inhibitor cocktail (Roche). Proteins were separated by 15% SDS-PAGE gels and transferred to a 0.22 μM PVDF membrane using Trans-Blot Turbo transfer system (Biorad). Transferred PVDF membranes containing the proteins were blocked by 5% BSA in TBS-T (0.05% Tween-20) for 1 h at room temperature. Histidine tag immunoblots were performed by incubating the membrane in mouse His-HRP antibodies for 1 h at room temperature, and β-actin immunoblots were performed by incubating the membranes with primary mouse anti-β-actin antibodies at room temperature for 40 min followed by HRP-conjugated anti-mouse IgG secondary antibodies for 1 h. Membranes were then developed using Amersham ECL Prime Western blotting detection reagent (GE Healthcare) and signals were detected with a ChemiDoc MP Imaging System (Bio-Rad)

Statistical analysis. Data are presented as mean and standard deviation of multiple biological replicates or independent experiments as indicated. Statistical analysis was performed using GraphPad Prism 9 software. Results were tested for significance using one-way ANOVA with Bonferroni's multiple comparisons test. Data were considered significant if *P<0.05, **P<0.01, ***P<0.001, ****p 0.0001.

### 8.2 Results and Discussion

The ability of TAT-hupa4 to inhibit inflammasome activation across multiple models of inflammasome activation was assessed.

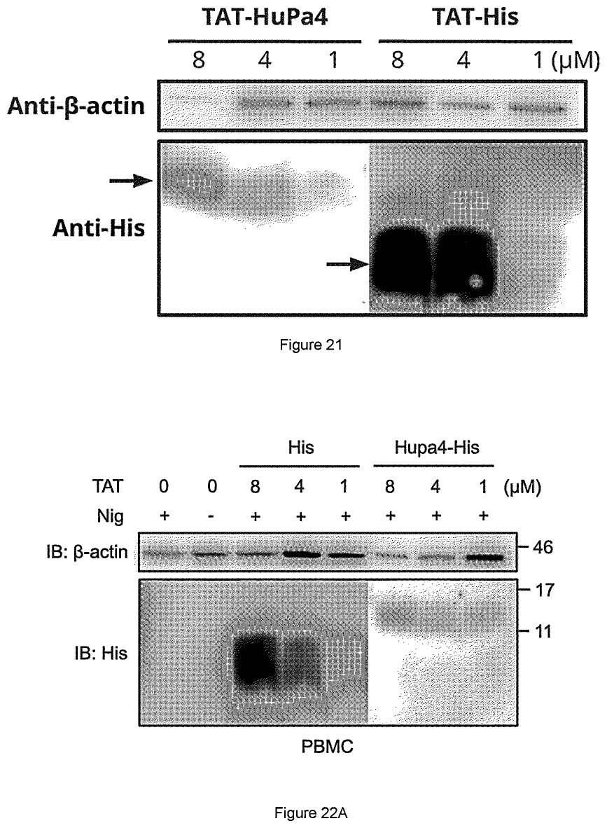

FIG. 21 demonstrates that Tat-hupa 4 is delivered into PBMCs.

### NLRP3 Inflammasome Activation by Nigericin in PBMCs

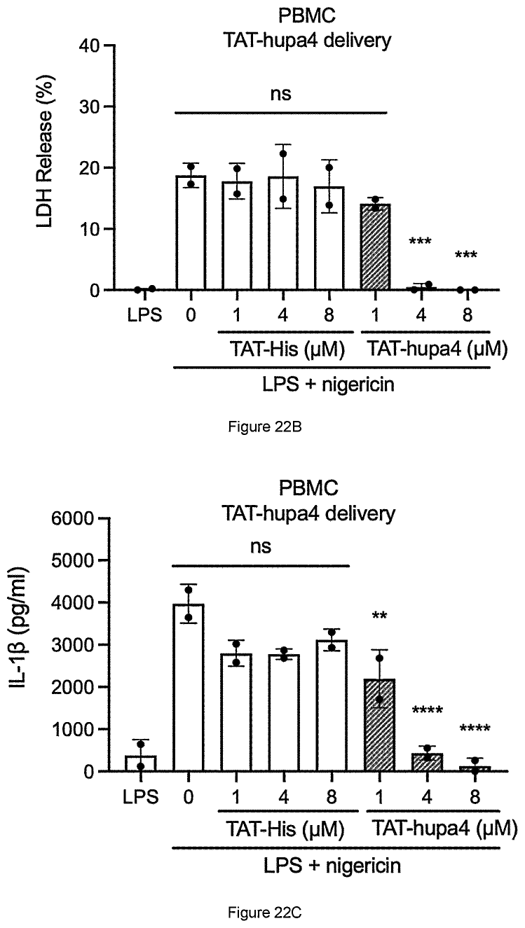

FIG. 22A demonstrates that TAT-hupa4 is delivered into LPS-primed PBMCs stimulated with nigericin.

FIG. 22B and FIG. 22C demonstrate that LDH release and secretion of IL-1β are significantly reduced in PBMCs stimulated with nigericin following TAT-hupa4 delivery compared to the control. LDH release and secretion of IL-1β are shown to be reduced in a TAT-hupa4 concentration-dependent manner.

These data show that TAT-hupa4 reduces inflammasome activation, cell death and IL-1β secretion, in a model of NLRP3 inflammasome activation in primary human immune cells.

### α-Synuclein-Induced Inflammasome Activation in PBMCs as a Parkinson's Disease Model

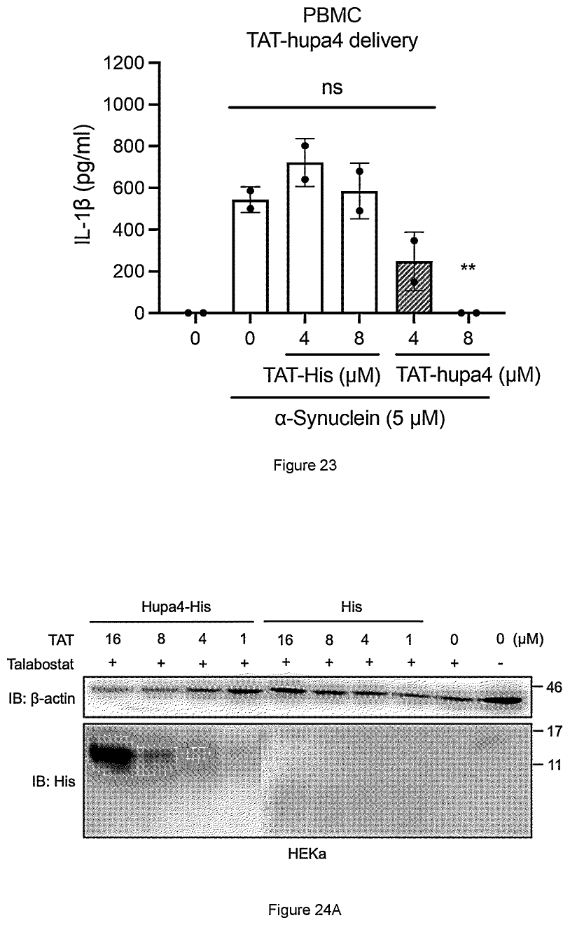

FIG. 23 demonstrates that secretion of IL-1β is significantly reduced in PBMCs stimulated with α-synuclein following TAT-hupa4 delivery compared to the control. Secretion of IL-1β is shown to be reduced in a TAT-hupa4 concentration-dependent manner.

These data show that TAT-hupa4 reduces inflammasome activation and IL-1β secretion in an α-synuclein-induced inflammasome activation in PBMCs as a Parkinson's disease in vitro model.

### NLRP1 Inflammasome Activation by Talabostat in HEKa

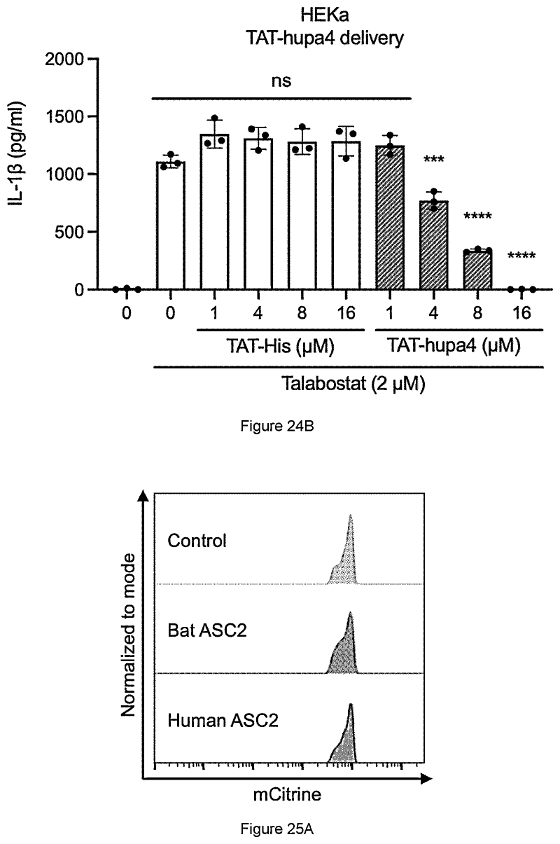

FIG. 24A demonstrates that TAT-hupa4 is delivered into HEKa (human epidermal keratinocytes, adult) cells stimulated by talabostat.

FIG. 24B demonstrates that secretion of IL-1β is significantly reduced in HEKa cells stimulated with talabostat following TAT-hupa4 delivery compared to the control. Secretion of IL-1β is shown to be reduced in a TAT-hupa4 concentration-dependent manner.

These data show that TAT-hupa4 reduces inflammasome activation and IL-1β secretion in a model of NLRP1 inflammasome activation in primary human skin cells.

Overall, these data show that TAT-hupa4 is capable of reducing inflammasome activation across various models of inflammasome activation (e.g. NLRP3 inflammasome activation by nigericin in PBMCs, α-synuclein-induced Parkinson's disease model in PBMCs, NLRP1 inflammasome activation by talabostat in HEKa).

### Example 9: Ability of Bat ASC2 to Inhibit Inflammasome Activation in N/TERT-1 Keratinocytes

### 9.1 Materials and Methods

Cell culture. Immortalized N/TERT-1 keratinocyte were cultured in KSFM media (Gibco) supplemented with 30 g/ml BPE, 0.1 ng/ml rEGF, and 0.3 mM calcium chloride.

Generation of immortalized N/TERT-1 keratinocyte stable cells. GP2-293 cells stably expressing gag and pol proteins, were co-transfected with pVSV-G envelope vector and mCitrine-only empty vector, bat or human ASC2-mCitirine pQCXIH vector for 48 h. Supernatants containing retroviruses were collected, centrifuged at 500 g for 10 min. N/TERT-1 cells were transduced with low MOI of retrovirus for 24 h to obtain a single viral copy per transduced cell. 72 h post-transduction, cells were selected by 20 μg/mL Hygromycin B antibiotic for at least 1 week to ensure stable expression. The stable cells were FACS-sorted for equal fluorescent intensity to ensure the same expression levels across cell lines.

Inflammasome activation assay. N/TERT-1 cells in KSFM without antibiotics were seeded at 1.8×10⁵/mL into 96-well plates overnight. Cells were treated with various concentrations (0, 20, 200 μM) of talabostat dissolved in DMSO for 24 h. DMSO concentration is controlled at 0.5% for all treated wells. The superatant was then harvested for LDH release assay and IL-1β detection by ELISA.

### 9.2 Results and Discussion

The ability of bat ASC2 to inhibit inflammasome activation in N/TERT-1 keratinocytes as compared to human ASC2 was investigated.

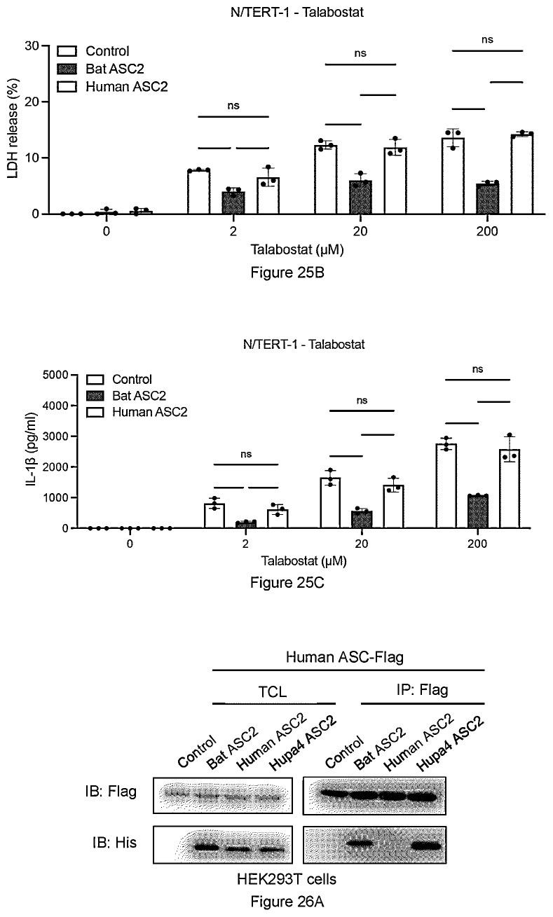

FIG. 25A shows immortalized N/TERT-1 keratinocytes stably expressing mid mCitrine (control), bat or human ASC2-mCitrine at a comparable level.

FIG. 25B demonstrates that bat ASC2 reduces cell death in talabostat-induced NLRP1 inflammasome activation in N/TERT-1 keratinocytes to a greater extent than human ASC2.

FIG. 25C demonstrates that bat ASC2 reduces IL-1β secretion in talabostat-induced NLRP1 inflammasome activation in N/TERT-1 keratinocytes to a greater extent than human ASC2.

These data show that bat ASC2 is significantly more potent in inhibiting NLRP1 inflammasomes than human ASC2 in human skin cells.

### Example 10: ASC2 is Co-Immunoprecipitated with ASC

### 10.1 Materials and Methods

Co-immunoprecipitation. HEK293T cells were transfected in 6-well plate with plasmids encoding His-tagged empty vector (His alone), bat ASC2, human ASC2, and Hupa4 ASC2 along with Flag-tagged human or bat ASC. 3 μg human ASC2, in contrast to 0.25 μg control, bat or hupa4 ASC2, were transfected to normalize the expression. At 48 h post-transfection, cells were lysed in IP lysis buffer (Pierce) according to manufacturer's protocol and centrifuged at 500 g for 10 minutes at 4° C. The supernatants were subjected to immunoprecipitation by incubation for 16 hours at 4° C. with anti-Flag (Sigma) incubated magnetic beads, followed by washing three times with TBS containing 0.05% Tween 20 and once with purified water. Bound proteins were separated by 15% SDS-PAGE, followed by immunoblotting analysis with the indicated antibodies.

### 10.2 Results and Discussion

To determine if there is an interaction between ASC and ASC2, co-immunoprecipitation (co-IP) was performed with HEK293T cells transfected with human or bat ASC, and control, bat, human or hupa4 ASC2.

FIG. 26A demonstrates that bat ASC2 and hupa4 ASC2 interact strongly with human ASC compared to human ASC2.

FIG. 26B similarly demonstrates that bat ASC2 and hupa4 ASC2 interact with bat ASC more strongly than human ASC2.

These data show that there is a physical interaction between bat/hupa4 ASC2 and ASC, and indicate that the stronger suppression of inflammasome activation by bat/hupa4 ASC compared to human ASC2 is contributed by the more robust interaction with ASC.

### Example 11: ASC2 of Non-Primate and Non-Bat Species Shows Variable Function, from Inhibitory, Neutral to Activating Effect on Inflammasome Activation

### 11.1 Materials and Methods

Plasmids. ASC2 sequences of 16 non-primate non-bat species were gene synthesized (IDT) and cloned into pQCXIH-LSSmOrange vector (Clontech). All constructs were prepared with endotoxin-free plasmid kits (Omega Bio-tek).

Transfection. HEK293T cells were seeded at 3×10⁵/mL in 96-well plate overnight. The following day, the cells were transfected with 12.5 ng mCitrine (negative control) or human NLRP3-mCitrine, 1.5 ng human ASC-mPlum, 50 ng LSSmOrange (control) or ASC2-LSSmOrange of 16 non-primates non-bats species, and 186 ng filler plasmid per well. 48 h post-transfection, cells were harvested for flow cytometry analysis.

### 11.2 Results and Discussion

To determine the function of ASC2 of non-primate and non-bat species, reconstituted HEK293T cells were transfected with bat ASC2, human ASC2, Hupa4 ASC2 or ASC2 of 16 non-primate non-bat species.

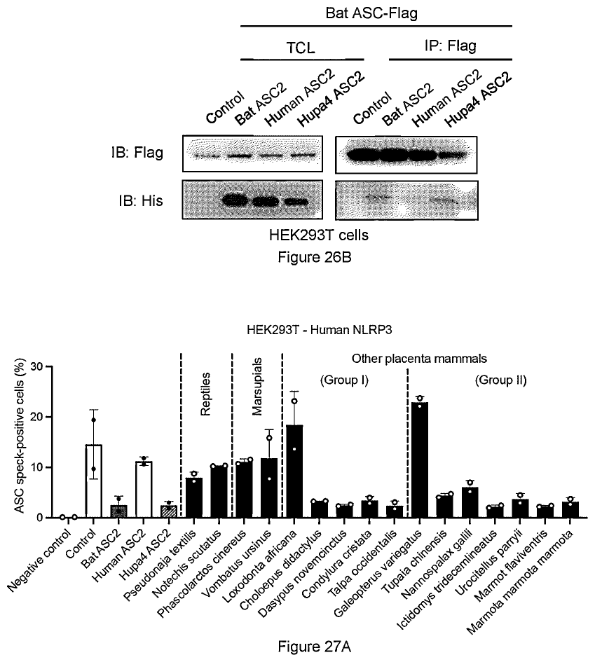

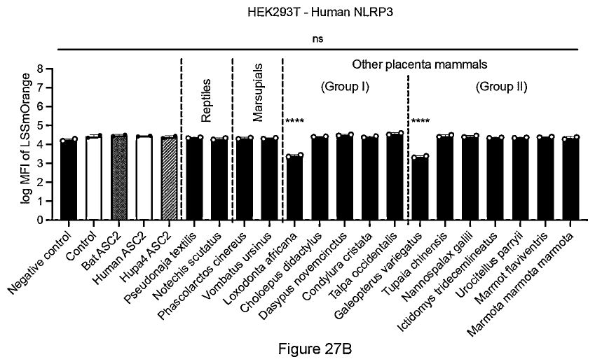

FIG. 27A demonstrate that these 16 ASC2 proteins show variable function, from inhibitory, neutral to activating effect on inflammasome activation.

FIG. 27B demonstrates that these ASC2 proteins show comparable expression level except the ASC2 from the two species that shows enhancing effect.

These data show that in contrast to inhibitory effect for bat ASC2 and non-inhibitory effect for primate ASC2, non-primate non-bat ASC2 exhibits variable function, from inhibitory, neutral to activating effect on inflammasome activation.

### REFERENCES

1. Broz, P. & Dixit, V. M. Inflammasomes: mechanism of assembly, regulation and signalling. Nat. Rev. Immunol. 16, 407-420, doi: 10.1038/nri.2016.58 (2016).
2. Guo, H., Callaway, J. B. & Ting, J. P. Inflammasomes: mechanism of action, role in disease, and therapeutics. Nat. Med. 21, 677-687, doi: 10.1038/nm.3893 (2015).
3. Mangan, M. S. J. et al. Targeting the NLRP3 inflammasome in inflammatory diseases. Nat. Rev. Drug Discov. 17, 688, doi: 10.1038/nrd.2018.149 (2018).
4. Brook, C. E. & Dobson, A. P. Bats as ‘special’ reservoirs for emerging zoonotic pathogens. Trends Microbiol., doi: 10.1016/j.tim.2014.12.004 (2015).
5. Wilkinson, G. S. & South, J. M. Life history, ecology and longevity in bats. Aging Cell 1, 124-131 (2002).
6. Ahn, M. et al. Dampened NLRP3-mediated inflammation in bats and implications for a special viral reservoir host. Nat Microbiol, doi: 10.1038/s41564-019-0371-3 (2019).
7. Ahn, M., Cui, J., Irving, A. T. & Wang, L. F. Unique Loss of the PYHIN Gene Family in Bats Amongst Mammals: Implications for Inflammasome Sensing. Sci. Rep. 6, 21722, doi: 10.1038/srep21722 (2016).
8. Le, H. T. & Harton, J. A. Pyrin- and CARD-only Proteins as Regulators of NLR Functions. Front. Immunol. 4, 275, doi: 10.3389/fimmu.2013.00275 (2013).
9. Stehlik, C. et al. The PAAD/PYRIN-only protein POP1/ASC2 is a modulator of ASC-mediated nuclear-factor-kappa B and pro-caspase-1 regulation. Biochem. J. 373, 101-113, doi: 10.1042/BJ20030304 (2003).
10. Kanneganti, T. D. The inflammasome: firing up innate immunity. Immunol. Rev. 265, 1-5, doi: 10.1111/imr. 12297 (2015).
11. Schroder, K. & Tschopp, J. The inflammasomes. Cell 140, 821-832, doi: 10.1016/j.cell.2010.01.040 (2010).
12. Hara, H. et al. Phosphorylation of the adaptor ASC acts as a molecular switch that controls the formation of speck-like aggregates and inflammasome activity. Nat. Immunol. 14, 1247-1255, doi: 10.1038/ni.2749 (2013).
13. Dick, M. S., Sborgi, L., Ruhl, S., Hiller, S. & Broz, P. ASC filament formation serves as a signal amplification mechanism for inflammasomes. Nat Commun 7, 11929, doi: 10.1038/ncomms11929 (2016).
14. von Moltke, J. et al. Rapid induction of inflammatory lipid mediators by the inflammasome in vivo. Nature 490, 107-111, doi: 10.1038/nature11351 (2012).
15. Venegas, C. et al. Microglia-derived ASC specks cross-seed amyloid-beta in Alzheimer's disease. Nature 552, 355-361, doi: 10.1038/nature25158 (2017).
16. Franklin, B. S. et al. The adaptor ASC has extracellular and ‘prionoid’ activities that propagate inflammation. Nat. Immunol. 15, 727-737, doi: 10.1038/ni.2913 (2014).
17. Baroja-Mazo, A. et al. The NLRP3 inflammasome is released as a particulate danger signal that amplifies the inflammatory response. Nat. Immunol. 15, 738-748, doi: 10.1038/ni.2919 (2014).
18. Strowig, T., Henao-Mejia, J., Elinav, E. & Flavell, R. Inflammasomes in health and disease. Nature 481, 278-286, doi: 10.1038/nature10759 (2012).
19. Youm, Y. H. et al. Canonical Nlrp3 inflammasome links systemic low-grade inflammation to functional decline in aging. Cell Metab 18, 519-532, doi: 10.1016/j.cmet.2013.09.010 (2013).
20. McAuley, J. L. et al. Activation of the NLRP3 inflammasome by IAV virulence protein PB1-F2 contributes to severe pathophysiology and disease. PLOS Pathog. 9, e1003392, doi: 10.1371/journal.ppat. 1003392 (2013).
21. Swanson, K. V., Deng, M. & Ting, J. P. The NLRP3 inflammasome: molecular activation and regulation to therapeutics. Nat. Rev. Immunol., doi: 10.1038/s41577-019-0165-0 (2019).
22. Dinarello, C. A., Simon, A. & van der Meer, J. W. Treating inflammation by blocking interleukin-1 in a broad spectrum of diseases. Nat. Rev. Drug Discov. 11, 633-652, doi: 10.1038/nrd3800 (2012).
23. Mackenzie, S. H., Schipper, J. L. & Clark, A. C. The potential for caspases in drug discovery. Curr Opin Drug Discov Devel 13, 568-576 (2010).
24. Boxer, M. B. et al. A highly potent and selective caspase 1 inhibitor that utilizes a key 3-cyanopropanoic acid moiety. ChemMedChem 5, 730-738, doi: 10.1002/cmdc.200900531 (2010).
25. Coll, R. C. et al. A small-molecule inhibitor of the NLRP3 inflammasome for the treatment of inflammatory diseases. Nat. Med. 21, 248-255, doi: 10.1038/nm.3806 (2015).
26. Tapia-Abellan, A. et al. MCC950 closes the active conformation of NLRP3 to an inactive state. Nat. Chem. Biol. 15, 560-564, doi: 10.1038/s41589-019-0278-6 (2019).
27. Coll, R. C. et al. MCC950 directly targets the NLRP3 ATP-hydrolysis motif for inflammasome inhibition. Nat. Chem. Biol. 15, 556-559, doi: 10.1038/s41589-019-0277-7 (2019).
28. Sand, J. et al. Expression of inflammasome proteins and inflammasome activation occurs in human, but not in murine keratinocytes. Cell Death Dis. 9, 24, doi: 10.1038/s41419-017-0009-4 (2018).
29 Zhong, F. L. et al. Germline NLRP1 Mutations Cause Skin Inflammatory and Cancer Susceptibility Syndromes via Inflammasome Activation. Cell 167, 187-202 e117, doi: 10.1016/j.cell.2016.09.001 (2016).
30. Grandemange, S. et al. A new autoinflammatory and autoimmune syndrome associated with NLRP1 mutations: NAIAD (NLRP1-associated autoinflammation with arthritis and dyskeratosis). Ann. Rheum. Dis. 76, 1191-1198, doi: 10.1136/annrheumdis-2016-210021 (2017).
31. Saresella, M. et al. The NLRP3 and NLRP1 inflammasomes are activated in Alzheimer's disease. Mol. Neurodegener. 11, 23, doi: 10.1186/s13024-016-0088-1 (2016).
32. Tan, M. S. et al. Amyloid-beta induces NLRP1-dependent neuronal pyroptosis in models of Alzheimer's disease. Cell Death Dis. 5, e1382, doi: 10.1038/cddis.2014.348 (2014).
33. Paulin, N. et al. Double-Strand DNA Sensing Aim2 Inflammasome Regulates Atherosclerotic Plaque Vulnerability. Circulation 138, 321-323, doi: 10.1161/CIRCULATIONAHA. 117.033098 (2018).
34. Denes, A. et al. AIM2 and NLRC4 inflammasomes contribute with ASC to acute brain injury independently of NLRP3. Proc. Natl. Acad. Sci. U.S.A 112, 4050-4055, doi: 10.1073/pnas. 1419090112 (2015).
35. Zhang, H. et al. AIM2 Inflammasome Is Critical for Influenza-Induced Lung Injury and Mortality. J. Immunol. 198, 4383-4393, doi: 10.4049/jimmunol. 1600714 (2017).
36. Atianand, M. K. & Harton, J. A. Uncoupling of Pyrin-only protein 2 (POP2)-mediated dual regulation of NF-kappaB and the inflammasome. J. Biol. Chem. 286, 40536-40547, doi: 10.1074/jbc.M111.274290 (2011).
37. de Almeida, L. et al. The PYRIN Domain-only Protein POP1 Inhibits Inflammasome Assembly and Ameliorates Inflammatory Disease. Immunity 43, 264-276, doi: 10.1016/j.immuni.2015.07.018 (2015).
38. Ahn, M. et al. Dampened NLRP3-mediated inflammation in bats and implications for a special viral reservoir host. Nat Microbiol 4, 789-799, doi: 10.1038/s41564-019-0371-3 (2019).
39. Guidotti, G., Brambilla, L. & Rossi, D. Cell-Penetrating Peptides: From Basic Research to Clinics. Trends Pharmacol. Sci. 38, 406-424, doi: 10.1016/j.tips.2017.01.003 (2017).
40. Vives, E., Brodin, P. & Lebleu, B. A truncated HIV-1 Tat protein basic domain rapidly translocates through the plasma membrane and accumulates in the cell nucleus. J. Biol. Chem. 272, 16010-16017, doi: 10.1074/jbc.272.25.16010 (1997).
41. Martinon, F., Petrilli, V., Mayor, A., Tardivel, A. & Tschopp, J. Gout-associated uric acid crystals activate the NALP3 inflammasome. Nature 440, 237-241, doi: 10.1038/nature04516 (2006).
42. Chakrabarti, A. et al. RNase L Activates the NLRP3 Inflammasome during Viral Infections. Cell Host Microbe 17, 466-477, doi: 10.1016/j.chom.2015.02.010 (2015).
43. Ichinohe, T., Pang, I. K. & Iwasaki, A. Influenza virus activates inflammasomes via its intracellular M2 ion channel. Nat. Immunol. 11, 404-410, doi: 10.1038/ni. 1861 (2010).
44. Wang, W. et al. Zika virus infection induces host inflammatory responses by facilitating NLRP3 inflammasome assembly and interleukin-1beta secretion. Nat Commun 9, 106, doi: 10.1038/s41467-017-02645-3 (2018).
45. Chua, K. B. et al. A previously unknown reovirus of bat origin is associated with an acute respiratory disease in humans. Proc. Natl. Acad. Sci. U.S.A 104, 11424-11429, doi: 10.1073/pnas.0701372104 (2007).
46. Natarajan, A., Ghose, R. & Hill, J. M. Structure and dynamics of ASC2, a pyrin domain-only protein that regulates inflammatory signaling. J. Biol. Chem. 281, 31863-31875, doi: 10.1074/jbc.M605458200 (2006).
47. Choi, J. Y. et al. Crystal structure of human POP1 and its distinct structural feature for PYD domain. Biochem. Biophys. Res. Commun. 460, 957-963, doi: 10.1016/j.bbrc.2015.03.134 (2015).
48. Lu, A. et al. Unified polymerization mechanism for the assembly of ASC-dependent inflammasomes. Cell 156, 1193-1206, doi: 10.1016/j.cell.2014.02.008 (2014).
49. Crameri, G. et al. Establishment, immortalisation and characterisation of pteropid bat cell lines. PLOS One 4, e8266, doi: 10.1371/journal.pone.0008266 (2009).
50. Sadler, A. J., Latchoumanin, O., Hawkes, D., Mak, J. & Williams, B. R. An antiviral response directed by PKR phosphorylation of the RNA helicase A. PLOS Pathog. 5, e1000311, doi: 10.1371/journal.ppat. 1000311 (2009).
51. Zhou, P. et al. Unlocking bat immunology: establishment of Pteropus alecto bone marrow-derived dendritic cells and macrophages. Sci. Rep. 6, 38597, doi: 10.1038/srep38597 (2016).
52. Katoh, K. & Standley, D. M. MAFFT multiple sequence alignment software version 7: improvements in performance and usability. Mol. Biol. Evol. 30, 772-780, doi: 10.1093/molbev/mst010 (2013).
53. Guindon, S. et al. New algorithms and methods to estimate maximum-likelihood phylogenies: assessing the performance of PhyML 3.0. Syst. Biol. 59, 307-321, doi: 10.1093/sysbio/syq010 (2010).
54. Derossi, D., Joliot, A. H., Chassaing, G. & Prochiantz, A. The third helix of the Antennapedia homeodomain translocates through biological membranes. J. Biol. Chem. 269, 10444-10450 (1994).
55. Joliot, A., Pernelle, C., Deagostini-Bazin, H. & Prochiantz, A. Antennapedia homeobox peptide regulates neural morphogenesis. Proc. Natl. Acad. Sci. U.S.A 88, 1864-1868, doi: 10.1073/pnas.88.5.1864 (1991).
56. Allolio, C. et al. Arginine-rich cell-penetrating peptides induce membrane multilamellarity and subsequently enter via formation of a fusion pore. Proc. Natl. Acad. Sci. U.S.A 115, 11923-11928, doi: 10.1073/pnas. 1811520115 (2018).
57. De Coupade, C. et al. Novel human-derived cell-penetrating peptides for specific subcellular delivery of therapeutic biomolecules. Biochem. J. 390, 407-418, doi: 10.1042/BJ20050401 (2005).
58. Morris, M. C., Deshayes, S., Heitz, F. & Divita, G. Cell-penetrating peptides: from molecular mechanisms to therapeutics. Biol. Cell 100, 201-217, doi: 10.1042/BC20070116 (2008).
59. Naldini, L. Gene therapy returns to centre stage. Nature 526, 351-360, doi: 10.1038/nature15818 (2015).
60. Brown, T. D., Whitehead, K. A. & Mitragotri, S. Materials for oral delivery of proteins and peptides. Nature Reviews Materials 5, 127-148 (2020).
61. Dombrowski, Y. et al. Cytosolic DNA triggers inflammasome activation in keratinocytes in psoriatic lesions. Sci. Transl. Med. 3, 82ra38, doi: 10.1126/scitranslmed.3002001 (2011).
62. Kerur, N. et al. cGAS drives noncanonical-inflammasome activation in age-related macular degeneration. Nat. Med. 24, 50-61, doi: 10.1038/nm.4450 (2018).
63. Chi, W. et al. Caspase-8 promotes NLRP1/NLRP3 inflammasome activation and IL-1beta production in acute glaucoma. Proc. Natl. Acad. Sci. U.S.A 111, 11181-11186, doi: 10.1073/pnas. 1402819111 (2014).
64. Tarallo, V. et al. DICER1 loss and Alu RNA induce age-related macular degeneration via the NLRP3 inflammasome and MyD88. Cell 149, 847-859, doi: 10.1016/j.cell.2012.03.036 (2012).
65. Kim, R. Y. et al. Role for NLRP3 Inflammasome-mediated, IL-1beta-Dependent Responses in Severe, Steroid-Resistant Asthma. Am. J. Respir. Crit. Care Med. 196, 283-297, doi: 10.1164/rccm.201609-1830OC (2017).
66. Besnard, A. G. et al. NLRP3 inflammasome is required in murine asthma in the absence of aluminum adjuvant. Allergy 66, 1047-1057, doi: 10.1111/j. 1398-9995.2011.02586.x (2011).
67. Mridha, A. R. et al. NLRP3 inflammasome blockade reduces liver inflammation and fibrosis in experimental NASH in mice. J. Hepatol. 66, 1037-1046, doi: 10.1016/j.jhep.2017.01.022 (2017).
68. Henao-Mejia, J. et al. Inflammasome-mediated dysbiosis regulates progression of NAFLD and obesity. Nature 482, 179-185, doi: 10.1038/nature10809 (2012).
69. Kodama, Y. & Hu, C. D. An improved bimolecular fluorescence complementation assay with a high signal-to-noise ratio. BioTechniques 49, 793-805, doi: 10.2144/000113519 (2010).
70. Ohashi, K., Kiuchi, T., Shoji, K., Sampei, K. & Mizuno, K. Visualization of cofilin-actin and Ras-Raf interactions by bimolecular fluorescence complementation assays using a new pair of split Venus fragments. BioTechniques 52, 45-50, doi: 10.2144/000113777 (2012).
71. Shyu, Y. J., Liu, H., Deng, X. & Hu, C. D. Identification of new fluorescent protein fragments for bimolecular fluorescence complementation analysis under physiological conditions. Bio Techniques 40, 61-66, doi: 10.2144/000112036 (2006).
72. Dixon, A. S. et al. NanoLuc Complementation Reporter Optimized for Accurate Measurement of Protein Interactions in Cells. ACS chemical biology 11, 400-408, doi: 10.1021/acschembio.5b00753 (2016).
73. Machleidt, T. et al. NanoBRET—A Novel BRET Platform for the Analysis of Protein-Protein Interactions. ACS chemical biology 10, 1797-1804, doi: 10.1021/acschembio.5b00143 (2015).
74. Thirukkumaran, O. M. et al. Improved HaloTag Ligand Enables BRET Imaging With NanoLuc. Front Chem 7, 938, doi: 10.3389/fchem.2019.00938 (2019).
75. Hara, H. et al. Phosphorylation of the adaptor ASC acts as a molecular switch that controls the formation of speck-like aggregates and inflammasome activity. Nature immunology 14, 1247-1255, doi: 10.1038/ni.2749 (2013).
76. Franklin, B. S. et al. The adaptor ASC has extracellular and ‘prionoid’ activities that propagate inflammation. Nature immunology 15, 727-737, doi: 10.1038/ni.2913 (2014).
77. Dick, M. S., Sborgi, L., Ruhl, S., Hiller, S. & Broz, P. ASC filament formation serves as a signal amplification mechanism for inflammasomes. Nature communications 7, 11929, doi: 10.1038/ncomms11929 (2016).
78. Li, Y. et al. Cryo-EM structures of ASC and NLRC4 CARD filaments reveal a unified mechanism of nucleation and activation of caspase-1. Proceedings of the National Academy of Sciences of the United States of America 115, 10845-10852, doi: 10.1073/pnas. 1810524115 (2018).
79. Lu, A. et al. Unified polymerization mechanism for the assembly of ASC-dependent inflammasomes. Cell 156, 1193-1206, doi: 10.1016/j.cell.2014.02.008 (2014).
80. Proell, M., Gerlic, M., Mace, P. D., Reed, J. C. & Riedl, S. J. The CARD plays a critical role in ASC foci formation and inflammasome signalling. The Biochemical journal 449, 613-621, doi: 10.1042/BJ20121198 (2013).
81. Pal, A. et al. Inhibition of NLRP3 inflvdammasome activation by cell-permeable stapled peptides. Scientific reports 9, 4913, doi: 10.1038/s41598-019-41211-3 (2019).
82. Chakraborty, S., Gonzalez, J., Edwards, K., Mallajosyula, V., Buzzanco, A. S., Sherwood, R., Buffone, C., Kathale, N., Providenza, S., Xie, M. M., et al. (2021). Proinflammatory IgG Fc structures in patients with severe COVID-19. Nature immunology 22, 67-73.
83. Larsen, M. D., de Graaf, E. L., Sonneveld, M. E., Plomp, H. R., Nouta, J., Hoepel, W., Chen, H. J., Linty, F., Visser, R., Brinkhaus, M., et al. (2020). Afucosylated IgG characterizes enveloped viral responses and correlates with COVID-19 severity. Science.
84. Pan, P., Shen, M., Yu, Z., Ge, W., Chen, K., Tian, M., Xiao, F., Wang, Z., Wang, J., Jia, Y., et al. (2021). SARS-COV-2 N protein promotes NLRP3 inflammasome activation to induce hyperinflammation. Nature communications 12, 4664.
85. Zheng, M., Karki, R., Williams, E. P., Yang, D., Fitzpatrick, E., Vogel, P., Jonsson, C. B., and Kanneganti, T. D. (2021). TLR2 senses the SARS-COV-2 envelope protein to produce inflammatory cytokines. Nature immunology.
86. Goh, G., Ahn, M., Zhu, F., Lee, L. B., Luo, D., Irving, A. T., Wang, LF., (2020). Complementary regulation of caspase-1 and IL-1β reveals additional mechanisms of dampened inflammation in bats. PNAS 117 (46) 28939-28949

1. A polypeptide comprising or consisting of an amino acid sequence having at least 50% amino acid sequence identity to SEQ ID NO:6, wherein the amino acid sequence of the polypeptide is non-identical to the amino acid sequence of a naturally-occurring protein, and wherein the polypeptide inhibits interaction between ASC and an interaction partner for ASC.

2. The polypeptide according to claim 1, wherein the polypeptide comprises one or more of: E or D at the position corresponding to position 10 of SEQ ID NO:6; R, K or H at the position corresponding position 37 of SEQ ID NO:6; C, R, K or H at the position corresponding position 61 of SEQ ID NO:6; and G at the position corresponding position 77 of SEQ ID NO:6.

3. The polypeptide according to claim 1 or claim 2, wherein the polypeptide comprises one or more of: E at the position corresponding to position 10 of SEQ ID NO:6; R at the position corresponding position 37 of SEQ ID NO:6; C at the position corresponding position 61 of SEQ ID NO:6; and G at the position corresponding position 77 of SEQ ID NO:6.

4. The polypeptide according to any one of claims 1 to 3, wherein the polypeptide comprises E or D at the position corresponding to position 10 of SEQ ID NO:6; R, K or H at the position corresponding position 37 of SEQ ID NO:6; C, R, K or H at the position corresponding position 61 of SEQ ID NO:6; and G at the position corresponding position 77 of SEQ ID NO:6.

5. The polypeptide according to any one of claims 1 to 4, wherein the polypeptide comprises E at the position corresponding to position 10 of SEQ ID NO:6; R at the position corresponding position 37 of SEQ ID NO: 6; C at the position corresponding position 61 of SEQ ID NO:6; and G at the position corresponding position 77 of SEQ ID NO:6.

6. The polypeptide according to any one of claims 1 to 5, wherein the polypeptide comprises or consists of the amino acid sequence of SEQ ID NO:6.

7. The polypeptide according to any one of claims 1 to 6, wherein the polypeptide further comprises a cell-penetrating peptide.

8. A composition comprising a polypeptide according to any one of claims 1 to 7, encapsulated in, or immobilised on, a nanoparticle, liposome, nanogel or nanolipogel.

9. A nucleic acid encoding a polypeptide according to any one of claims 1 to 7.

10. An expression vector, comprising a nucleic acid according to claim 9.

11. A cell comprising a polypeptide according to any one of claims 1 to 7, a composition according to claim 8, a nucleic acid according to claim 9, or an expression vector according to claim 10.

12. A method for producing a polypeptide according to any one of claims 1 to 7, comprising culturing a cell comprising a nucleic acid according to claim 9, or an expression vector according to claim 10, under conditions suitable for expression of the polypeptide by the cell.

13. A pharmaceutical composition comprising a polypeptide according to any one of claims 1 to 7, a composition according to claim 8, a nucleic acid according to claim 9, an expression vector according to claim 10, or a cell according to claim 11, and a pharmaceutically acceptable carrier, diluent, excipient or adjuvant.

14. A polypeptide according to any one of claims 1 to 7, a composition according to claim 8, a nucleic acid according to claim 9, an expression vector according to claim 10, a cell according to claim 11, or a pharmaceutical composition according to claim 13, for use in a method of medical treatment or prophylaxis.

15. A polypeptide according to any one of claims 1 to 7, a composition according to claim 8, a nucleic acid according to claim 9, an expression vector according to claim 10, a cell according to claim 11, or a pharmaceutical composition according to claim 13, for use in a method of treating or preventing a disease or condition in which inflammasome activity is pathologically-implicated.

16. Use of a polypeptide according to any one of claims 1 to 7, a composition according to claim 8, a nucleic acid according to claim 9, an expression vector according to claim 10, a cell according to claim 11, or a pharmaceutical composition according to claim 13, in the manufacture of a medicament for treating or preventing a disease or condition in which inflammasome activity is pathologically-implicated.

17. A method of treating or preventing a disease or condition in which inflammasome activity is pathologically-implicated, comprising administering a therapeutically- or prophylactically-effective amount of a polypeptide according to any one of claims 1 to 7, a composition according to claim 8, a nucleic acid according to claim 9, an expression vector according to claim 10, a cell according to claim 11, or a pharmaceutical composition according to claim 13, to a subject in need thereof.

18. The polypeptide, composition, nucleic acid, expression vector, cell or pharmaceutical composition for use according to claim 15, the use according to claim 16, or the method according to claim 17, wherein the disease or condition in which inflammasome activity is pathologically-implicated is selected from: an inflammatory disease, a chronic inflammatory disease, a neurodegenerative disease, Alzheimer's disease, Parkinson's disease, an autoimmune disease, multiple sclerosis, rheumatoid arthritis, systemic lupus erythematosus (SLE), an autoinflammatory disease, cryopyrin-associated periodic syndrome (CAPS), familial cold autoinflammatory syndrome (FCAS), Muckle-Wells syndrome (MWS), neonatal onset multisystemic inflammatory disease (NOMID), chronic infantile neurologic cutaneous articular (CINCA), a cancer, myelodysplastic syndrome, a cardiovascular disease, atherosclerosis, acute myocardial infarction, stroke, hypertension, an ocular disease, glaucoma, acute glaucoma, macular degeneration, age-related macular degeneration, a respiratory disease, asthma, severe steroid-resistant asthma, chronic obstructive pulmonary disease (COPD), respiratory disease cause by viral infection, gout, a metabolic disease, obesity-induced insulin resistance, diabetes, type 1 diabetes, type 2 diabetes, non-alcoholic fatty liver disease (NAFLD), silicosis, crystal nephropathy, a skin disease, acne, atopic dermatitis, psoriasis, vitiligo, contact hypersensitivity, inflammatory disease caused by viral infection, inflammatory bowel disease, graft-versus-host disease and traumatic brain injury.

19. A method for inhibiting an inflammasome, comprising introducing into a cell, in vitro or in vivo:
(i) a polypeptide comprising or consisting of an amino acid sequence having at least 50% amino acid sequence identity to SEQ ID NO: 6 which inhibits interaction between ASC and an interaction partner for ASC;
(ii) polypeptide according to (i), wherein the polypeptide further comprises a cell-penetrating peptide;
(iii) a composition comprising a polypeptide according to (i) or (ii), encapsulated in, or immobilised on, a nanoparticle, liposome, nanogel or nanolipogel;
(iv) a nucleic acid encoding a polypeptide according to (i) or (ii); or
(v) an expression vector comprising a nucleic acid according to (iv).

20. Use of:
(i) a polypeptide comprising or consisting of an amino acid sequence having at least 50% amino acid sequence identity to SEQ ID NO: 6 which inhibits interaction between ASC and an interaction partner for ASC;
(ii) polypeptide according to (i), wherein the polypeptide further comprises a cell-penetrating peptide;
(iii) a composition comprising a polypeptide according to (i) or (ii), encapsulated in, or immobilised on, a nanoparticle, liposome, nanogel or nanolipogel;
(iv) a nucleic acid encoding a polypeptide according to (i) or (ii); or
(v) an expression vector comprising a nucleic acid according to (iv);
in an in vitro or in vivo method for inhibiting an inflammasome.
